(19) United States
(12) Patent Application Publication
(10) Pub. No.: US 2026/0028328 A1
(43) Pub. Date: Jan. 29, 2026

(54) HETEROARYL DERIVATIVES AS DDRs INHIBITORS

(71) Applicant: CHIESI FARMACEUTICI S.P.A., Parma (IT)

(72) Inventors: Roberta MAZZUCATO, Parma (IT); Nicolò IOTTI, Parma (IT); Laura CARZANIGA, Parma (IT); Andrea RIZZI, Parma (IT); Eleonora GHIDINI, Parma (IT); Benjamin Paul WHITTAKER, Parma (IT); Annarita CIANCIUSI, Parma (IT); Robert Stuart Laurie CHAPMAN, Parma (IT); Zuzana HAMASOVA, Parma (IT)

(73) Assignee: CHIESI FARMACEUTICI S.P.A., Parma (IT)

(21) Appl. No.: 18/995,301

(22) PCT Filed: Jul. 18, 2023
(86) PCT No.: PCT/EP2023/069886

§ 371 Date: Jan. 16, 2025

(30) Foreign Application Priority Data
Jul. 19, 2022 (EP) 22185860.8

(51) Int. Cl. C07D 401/14; A61K 31/444; A61K 31/4985; A61K 31/506; A61K 31/519; A61K 31/5377; C07D 205/04; C07D 401/12; C07D 471/04; C07D 487/04

(52) U.S. Cl. CPC C07D 401/14; A61K 31/444; A61K 31/4985; A61K 31/506; A61K 31/519; A61K 31/5377; C07D 205/04; C07D 401/12; C07D 471/04; C07D 487/04

(57) ABSTRACT

The present invention relates to compounds of formula (I) inhibiting Discoidin Domain Receptors (DDR inhibitors), methods of preparing such compounds, intermediate compounds useful in such preparations, pharmaceutical compositions containing them and therapeutic use thereof. The compounds of the invention may be useful for instance in the treatment of many disorders associated with DDR mechanisms.

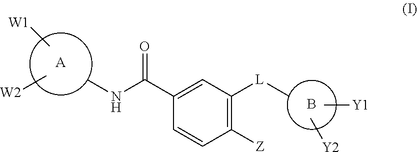

### FIELD OF THE INVENTION

The present invention relates to compounds inhibiting Discoidin Domain Receptors (DDR inhibitors), methods of preparing such compounds, intermediate compounds useful in such preparations, pharmaceutical compositions containing them and therapeutic use thereof.

The compounds of the invention may be useful for instance in the treatment of many disorders associated with DDR mechanisms.

### BACKGROUND OF THE INVENTION

Discoidin Domain Receptors (DDRs) are type I transmembrane receptor tyrosine kinase (RTKs). The DDR family comprises two distinct members, DDR1 and DDR2.

DDRs are unique receptors among the other members of the RTK superfamily, in that DDRs are activated by collagen whereas other members of the RTK superfamily are typically activated by soluble peptide-like growth factors (see Vogel, W. (1997) Mol. Cell 1, 13-23; Shrivastava A. Mol Cell. 1997; 1:25-34). Moreover, DDRs are unusual RTKs also because they form ligand-independent stable dimers that are non-covalently linked (see Noordeen, N. A. (2006) J. Biol. Chem. 281, 22744-22751; Mihai C. J Mol Biol. 2009; 385:432-445).

The DDR1 subfamily is composed of five membrane-anchored isoforms, and the DDR2 subfamily is represented by a single protein. The five DDR1 isoforms all have in common the extracellular and transmembrane domains but differ in the cytoplasmic region (see Valiathan, R. R. (2012) Cancer Metastasis Rev. 31, 295-321; Alves, F. (2001) FASEB J. 15, 1321-1323).

DDR receptor family has been found involved in a series of fibrotic diseases, such as pulmonary fibrosis, and in particular idiopathic pulmonary fibrosis (IDF). The first evidence for a protective role of DDR1 deletion in lung fibrosis was generated in 2006 by the research group of Dr. Vogel (see Avivi-Green C, Am J Respir Crit Care Med 2006; 174:420-427). The authors demonstrated that DDR1-null mice were largely protected against bleomycin (BLM)-induced injury. Furthermore, myofibroblast expansion and apoptosis were much lower in these animals compared with their wild-type counterparts. Absence of inflammation in knockout mice was confirmed by lavage cell count and cytokines ELISA. These results indicated that DDR1 expression is a prerequisite for the development of lung inflammation and fibrosis.

DDR2 deficiency or downregulation reduces bleomycin-induced lung fibrosis (see Zhao H, Bian H, Bu X, Zhang S, Zhang P, Yu J, et al Mol Ther 2016; 24:1734-1744). Zhao et al, demonstrated that DDR2 plays a critical role in the induction of fibrosis and angiogenesis in the lung, in particular that DDR2 synergizes with transforming growth factor (TGF)-β to induce myofibroblast differentiation. Furthermore, they showed that treatment of injured mice with specific siRNA against DDR2 exhibited therapeutic efficacy against lung fibrosis. In a second publication, Jia et al showed that mice lacking DDR2 are protected from bleomycin-induced lung fibrosis (see Jia S, Am J Respir Cell Mol Biol 2018; 59:295-305). In addition, DDR2-null fibroblasts are significantly more prone to apoptosis than wild-type fibroblasts, supporting a paradigm in which fibroblast resistance to apoptosis is critical for progression of fibrosis.

Some compounds have been described in the literature as DDR1 or DDR2 antagonists.

Of note, antagonizing the DDR receptors may be useful for the treatment of fibrosis and disease, disorder and conditions that result from fibrosis. Even more, antagonizing both receptors DDR1 and DDR2 may be particularly efficacious in the treatment of the above-mentioned disease, disorder and conditions.

Several efforts have been done in the past years to develop novel DDR1 and DDR2 receptor antagonists useful for the treatment of several diseases and some of those compounds have shown efficacy also in humans. However, there remains a potential for developing selective inhibitors of both receptors DDR1 and DDR2 useful for the treatment of diseases, disorders or conditions associated with a dysregulation of DDR receptors, in the respiratory field, in particular idiopathic pulmonary fibrosis (IPF), to be administered by the inhalation route and characterized by a good inhalatory profile, that corresponds to a good activity in the lung, a good lung retention and to a low metabolic stability in order to minimize the systemic exposure and correlated safety issues.

In this direction, a new series of compounds of general formula (I), as herein below reported, has been surprisingly found, which solves the problem of providing inhibitors for receptors DDR1 and DDR2 for administration by inhalation, which act as selective inhibitors of DDR1 and DDR2 receptors with respect to other human protein kinases. Such compounds show high potency, good inhalatory profile, low metabolic stability, low systemic exposure, improved safety and tolerability.

### SUMMARY OF THE INVENTION

In a first aspect the present invention relates to a compound of formula (I)

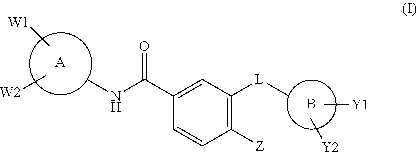

wherein
A is a ring selected from the group consisting of:

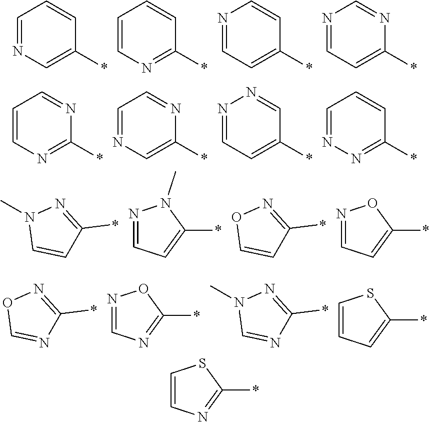

and phenyl, wherein [symbol] indicates a direct bond to NH;
W1 and W2 are substituents of ring A selected from the group consisting of hydrogen, halogen, (C₁-C₄)alkyl, (C₁-C₄)haloalkyl, (C₁-C₄)alkoxy, (C₁-C₄)haloalkoxy, R1R2N—(C₁-C₄)alkyl and (C₃-C₆)cycloalkyl, preferably selected from H, F, CH₃, OCH₃, OCF₃, CF₃, C(CH₃)₃, CH₂CH₃, C(CH₃)₂CF₃, OCF₂H, CHF₂, CH₂CF₃, CH₂N(CH₃)₂ and cyclopropyl;
Z is selected from the group consisting of (C₁-C₄)alkyl, (C₁-C₄)haloalkyl, (C₁-C₄)alkoxy, (C₁-C₄)haloalkoxy, (C₁-C₄)hydroxyalkyl and (C₃-C₆)cycloalkyl, preferably selected from CH₃, CF₂H, OCH₃, CH₂OH and cyclopropyl;
L is selected from the group consisting of

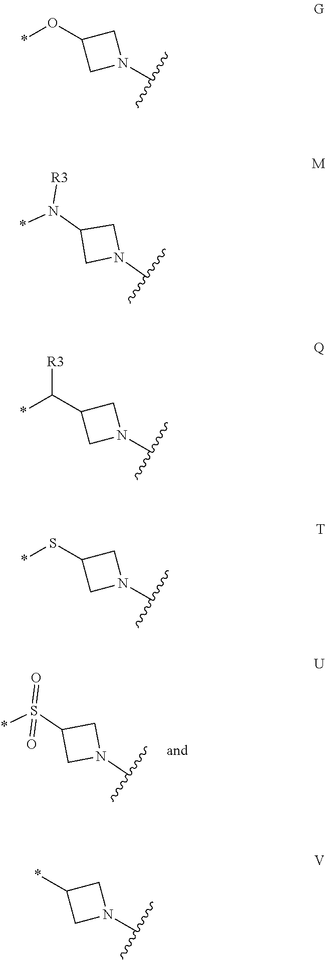

wherein [symbol] indicates a direct bond to the phenyl ring and

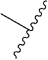

indicates a direct bond to B;
B is mono- or bi-cyclic heteroaryl ring, preferably selected from the group consisting of pyridinyl, pyrimidinyl, pyrazinyl, pyrazolo[1,5-a]pyrazinyl, 1H-pyrazolo[3,4-b]pyridinyl, pyrazolo[1,5-a]pyridinyl, pyrazolo[1,5-a]pyrimidinyl, imidazo[1,2-b]pyridazinyl, imidazo[1,2-a]pyrazinyl, imidazo[1,2-a]pyridinyl, thieno[3,2-d]pyrimidinyl, 1H-pyrrolo[2,3-b]pyridinyl and pyrazolyl;
Y1 and Y2 are substituents of ring B independently selected from the group consisting of hydrogen, (C₁-C₄)alkyl, (C₁-C₄)haloalkyl, (C₁-C₄)alkoxy, (C₁-C₄)alkoxy-(C₁-C₄)alkoxy, hydroxy-(C₁-C₄)alkoxy, hydroxy-(C₁-C₄)alkyl, (C₁-C₄)alkyl-heterocycloalkyl-(C₀-C₄)alkoxy, (C₁-C₄)haloalkoxy, halogen, cyano, cyano-(C₁-C₄)alkyl, cyano-(C₁-C₄)alkyl-heterocycloalkyl, CONR1R2, NHCOR1, NR1R2, R1R2N—(C₁-C₄)alkyl, heterocycloalkyl, (C₁-C₄)alkyl-heterocycloalkyl, heterocycloalkyl-(C₁-C₄)alkyl, (C₁-C₄)alkyl-heterocycloalkyl-carbonyl, (C₀-C₄)alkyl-heterocycloalkyl-carbonyl, (C₁-C₄)alkyl-phenyl and monocyclic (C₁-C₄)alkyl-heteroaryl, wherein Y1 is preferably selected from CONH₂, CF₂H, OCH₃, cyano, Br, NHCOCH₃, NH₂, 4-methylpiperazine-1-carbonyl, 4-methylpiperazin-1-yl and 1-methyl-1H-pyrazol-4-yl, and Y2 is preferably hydrogen;
R1 and R2 are independently selected from the group consisting of hydrogen, (C₁-C₄)alkyl, (C₁-C₄)hydroxyalkyl, (C₁-C₄)alkoxy(C₁-C₄)alkyl, (C₁-C₄)alkylamino-(C₁-C₄)alkyl, di-(C₁-C₄)alkylamino-(C₁-C₄)alkyl, optionally substituted (C₃-C₆)cycloalkyl, optionally substituted heterocycloalkyl and optionally substituted heterocycloalkyl-(C₁-C₄)alkoxy, wherein optional substituents are one or more and are selected from (C₁-C₄)alkyl, (C₁-C₄)alkoxy, (C₁-C₄)haloalkyl and (C₁-C₄)haloalkoxy; and wherein R1 and R2 are preferably independently selected from the group consisting of CH₂CH₂N(CH₃)₂, CH₂CH₂OCH₃, CH₃, CH₂CH₂OH, oxetan-3-yl and 3-hydroxycyclobutyl;
R3 is selected from hydrogen and (C₁-C₄)alkyl, preferably hydrogen and methyl;
   or a stereoisomer, tautomer, solvate and pharmaceutically acceptable salt thereof.

In a second aspect, the invention refers to a pharmaceutical composition comprising a compound of formula (I), or pharmaceutically acceptable salts thereof, in admixture with at least one or more pharmaceutically acceptable carrier and/or excipient.

In a third aspect, the invention refers to a compound of formula (I), or pharmaceutically acceptable salts thereof, or to a pharmaceutical composition comprising a compound of formula (I), or pharmaceutically acceptable salts thereof, for use as a medicament.

In a further aspect, the invention refers to a compound of formula (I), or pharmaceutically acceptable salts thereof, or to a pharmaceutical composition comprising a compound of formula (I), or pharmaceutically acceptable salts thereof, for use in preventing and/or treating a disease, disorder or condition associated with dysregulation of DDR.

In another aspect, the invention refers to a compound of formula (I), or pharmaceutically acceptable salts thereof, or to a pharmaceutical composition comprising a compound of formula (I), or pharmaceutically acceptable salts thereof, for use in preventing and/or treating fibrosis and/or diseases, disorders or conditions that involve fibrosis.

In yet another aspect, the invention refers to a compound of formula (I), or pharmaceutically acceptable salts thereof, or to a pharmaceutical composition comprising a compound of formula (I), or pharmaceutically acceptable salts thereof, for use in preventing and/or treating idiopathic pulmonary fibrosis (IPF).

In a further aspect, the invention refers to processes for the preparation of compounds of formula (I) and to intermediate compounds that are useful in their preparation.

### DETAILED DESCRIPTION OF THE INVENTION

### Definitions

Unless otherwise specified, the compounds of formula (I) of the present invention are intended to include stereoisomers, tautomers, solvates and pharmaceutically acceptable salts thereof.

Unless otherwise specified, the compounds of formula (I) of the present invention are intended to include the compounds of formula (I) and (Ic).

The term “pharmaceutically acceptable salts”, as used herein, refers to derivatives of compounds of formula (I) wherein the parent compound is suitably modified by converting any of the free acid or basic group, if present, into the corresponding addition salt with any base or acid conventionally intended as being pharmaceutically acceptable. Suitable examples of said salts may thus include mineral or organic acid addition salts of basic residues such as amino groups, as well as mineral or organic basic addition salts of acid residues such as carboxylic groups.

Cations of inorganic bases which can be suitably used to prepare salts comprise ions of alkali or alkaline earth metals, such as potassium, sodium, calcium or magnesium.

The salts obtained by reacting the main compound, functioning as a base, with an inorganic or organic acid comprise, for example, salts of hydrochloric acid, hydrobromic acid, sulfuric acid, phosphoric acid, methanesulfonic acid, camphorsulfonic acid, acetic acid, oxalic acid, maleic acid, fumaric acid, succinic acid and citric acid.

The term “stereoisomer” refers to isomers of identical constitution that differ in the arrangement of their atoms in space. Enantiomers and diastereomers are examples of stereoisomers.

The term “enantiomer” refers to one of a pair of molecular species that are mirror images of each other and are not superimposable.

The term “racemate” or “racemic mixture” refers to a composition composed of equimolar quantities of two enantiomeric species, wherein the composition is devoid of optical activity.

The term “halogen” or “halogen atoms” or “halo” as used herein includes fluorine, chlorine, bromine and iodine atom.

The term “(Cₓ-C_{y})alkyl”, wherein x and y are integers, refers to a straight or branched chain alkyl group having from x to y carbon atoms. Thus, when x is 1 and y is 4, for example, the term “(C₁-C₄)alkyl” comprises methyl, ethyl, n-propyl, isopropyl, n-butyl, isobutyl, sec-butyl, and t-butyl. Such groups are herein indicated also by their atoms arrangement, i.e. as CH₃, CH₂CH₃, etc.

The term “(Cₓ-C_{y})haloalkyl”, wherein x and y are integers, refers to a straight or branched chain alkyl group having from x to y carbon atoms, comprising at least one halogen substituent. Examples include CF₃, C(CH₃)₂CF₃ and OCF₂H.

The term “(Cₓ-C_{y})alkoxy”, wherein x and y are integers, refers to a straight or branched chain alkyl group having from x to y carbon atoms, comprising at least one oxygen atom, in particular, but not only, an oxygen atom directly linked to ring A or ring B or the phenyl ring, i.e. when W, Y or Z, respectively, are (C₁-C₄)alkoxy. Examples include OCH₃ and OCH₂CH₃.

The term “(Cₓ-C_{y})haloalkoxy”, wherein x and y are integers, refers to a “(Cₓ-C_{y})alkoxy comprising at least one halogen substituent. Examples include OCF₃ and OCF₂H.

The term “(Cₓ-C_{y})hydroxyalkyl”, wherein x and y are integers, refers to a (Cₓ-C_{y})alkyl comprising at least one hydroxy substituent. Examples include CH₂OH, CH(OH)CH₃ and CH₂CH₂OH.

The term “(Cₓ-C_{y})cycloalkyl” refers to a saturated hydrocarbon ring comprising a number of ring carbon atoms from x to y. Examples include cyclopropyl, cyclobutyl, cyclopentyl, cyclohexyl, and cycloheptyl.

The term “mono- or bi-cyclic heteroaryl ring” refers to a mono- or bi-cyclic aromatic group comprising 1 to 3 heteroatoms independently selected from N, S and O, and includes groups having two such monocyclic rings, or one such monocyclic ring and one monocyclic aryl ring, such as a phenyl ring, which are fused through a common bond or linked by a single bond. The mono- or bi-cyclic heteroaryl rings comprise pyrazolyl, furanyl, thiophenyl, oxazolyl, isoxazolyl, isothiazolyl, thiazolyl, pyridazinyl, imidazoyl, benzofuranyl, 1H-benzo[d]imidazolyl, 1H-indazolyl, benzothiophenyl, benzo[c]thiophenyl, quinazolinyl, pteridinyl, 1H-pyrazolo[5,1-c][1,2,4]triazolyl, pyrrolizinyl, indolizinyl, benzothiazolyl, pyrazolo[5,1-b]thiazolyl, 1H-imidazo[1,2-b]pyrazolyl, 1H-pyrazolo[3,4-b]pyridinyl, 1,6-dihydropyrrolo[2,3-b]pyrrolyl, 1,4-dihydropyrrolo[3,2-b]pyrrolyl, 4H-thieno[3,2-b]pyrrolyl, isobenzofuranyl, 1,2,4-triazolyl, 1,2,5 oxadiazolyl, 1,2,3 oxadiazolyl, 1,2,5 thiadiazolyl, 1,2,3 thiadiazolyl, tetrazolyl, 6H-furo[2,3-b]pyrrolyl, 6H-thieno[2,3-b]pyrrolyl, 4H-furo[3,2-b]pyrrolyl, benzo[d]isothiazolyl, thiazolo[4,5-b]pyridinyl, 1,3,5-triazinyl, 1,2,3,4-thiatriazolyl, 1,2,3,4-oxatriazolyl, 1,2,3,4-tetrazinyl, 1,2,4,5-tetrazinyl, 1,2,3,5-tetrazinyl, 1H-imidazo[4,5-b]pyridinyl, 7H-purinyl, 1H-pyrrolyl, 1-methyl-1H-pyrrolyl, 1-methyl-1H-1,2,4-triazolyl, 1-methyl-1H-tetrazolyl, pyridinyl, pyrimidinyl, pyridazinyl, imidazolyl, imidazo[1,2-b]pyridazinyl, pyrazolo[1,5-a]pyrazinyl, imidazo[1,2-a]pyrazinyl, imidazo[1,2-a]pyrazinyl, imidazo[1,2-a]pyridinyl, pyrazolo[1,5-a]pyridinyl, thieno[3,2-d]pyrimidinyl, 5H-pyrrolo[3,2-d]pyrimidinyl, 1H-pyrrolo[2,3-b]pyridinyl, 5,6-dihydro-8H-imidazo[2,1-c][1,4]oxazinyl, furo[3,2-d]pyrimidin-4-yl and pyrazolo[1,5-a]pyrimidinyl.

The term “heterocycloalkyl” refers to a saturated or partly unsaturated mono-, bi- or spiro-cyclic ring system of 3 to 12 ring atoms comprising one or more heteroatoms independently selected from N, S and O. Examples of heterocycloalkyl include piperazinyl, pyrrolidinyl, azetidinyl, morpholinyl and piperidinyl.

The term “spiro-cyclic ring system” refers to a saturated or partly unsaturated bi-cyclic ring system of 5 to 12 ring atoms, comprising one or more heteroatoms selected from N, S and O, wherein the two rings have only one common atom.

Any composite term, like “(C₁-C₄)alkyl-heterocycloalkyl-carbonyl”, should be intended as conventionally construed by the parts from which it derives, e.g. by a (C₁-C₄)alkyl, a heterocycloalkyl and a carbonyl group which are linked together in the indicated sequence, and wherein the carbonyl group carbon atom is the point of attachment to the residual part of the compound of formula (I).

When referring to substituents, a dash (“-”) that is not between two letters, words, or symbols is meant to represent the point of attachment for such substituents.

The carbonyl group is herein preferably represented as CO, as an alternative to the other common representations such as —C(O)—, —CO—, —(CO)— or —C(═O)—.

Whenever basic amino groups are present in the compounds of formula (I), physiologically acceptable anions may be present, selected among chloride, bromide, iodide, trifluoroacetate, formate, sulfate, phosphate, methanesulfonate, nitrate, maleate, acetate, citrate, fumarate, tartrate, oxalate, succinate, benzoate, p-toluenesulfonate, pamoate and naphthalene disulfonate. Likewise, in the presence of acidic groups, corresponding physiological cations may be present as well, for instance including alkaline or alkaline earth metal ions.

The term “Ki” indicates the dissociation constant for the enzyme-inhibitor complex, expressed in molar units. It is an indicator of the binding affinity between inhibitor and DDR1 or DDR2 receptors.

As above indicated, the present invention refers to a series of compounds represented by the general formula (I) as herein below described in detail, which are endowed with an inhibitory activity on receptors DDR1 and DDR2. Antagonizing receptors DDR1 and DDR2 can be particularly effective in the treatment of those diseases where the DDR receptors play a role, such as fibrosis and any other disease, disorder and condition related to fibrosis.

Indeed, as detailed in the experimental part below, the compounds of formula (I) of the present invention are able to act as inhibitors of both DDR1 and DDR2 receptors in a substantive and effective way. In particular, Table 2 further below shows that for representative compounds of the present invention the inhibitory activity against either DDR1 and DDR2 receptors is lower than 100 nM in the binding assay (expressed as Ki). This confirms that the compounds of formula (I) are able to inhibit the two isoforms of DDR receptor mainly involved in fibrosis and diseases resulting from fibrosis. Accordingly, the compounds of formula (I) can be used in the treatment of fibrosis, in particular pulmonary fibrosis, when DDR1 and DDR2 are involved.

As indicated in the experimental part, comparative examples section, in particular in Table 3, conversely to comparative compound C1, characterized by having a —CO— linker between L and heteroaryl ring B, the presence of a direct link between L and heteroaryl ring B in the compounds of the present invention unexpectedly and remarkably determines a relevant increase in the inhibitory activity on the DDR1 and DDR2 receptors.

Advantageously, the compounds of the present invention are endowed with a very high potency and could be administered in human at a lower dosage respect to the compounds of the prior art, thus reducing the adverse events that typically occur administering higher dosages of drug.

In addition to being notably potent with respect to their inhibitory activity on both receptors DDR1 and DDR2, the compounds of the present invention are also characterized by being selective inhibitors of DDR1 and DDR2 receptors with respect to other human protein kinases, by a good inhalatory profile, that allows to act effectively on the lung compartment and have, at the same time, a low metabolic stability, that allows to minimize the drawbacks associated with the systemic exposure, such as safety and tolerability issues.

Therefore, the compounds of the present invention may be particularly appreciated when looking at suitable and efficacious compounds useful for the treatment of fibrosis, in particular idiopathic pulmonary fibrosis, administered by the inhalation route and characterized by a good inhalatory profile, that corresponds to a good activity on the lung, a good lung retention and a low metabolic stability, that minimizes the systemic exposure and correlated safety issues. Accordingly, the present invention relates to a compound of formula (I)

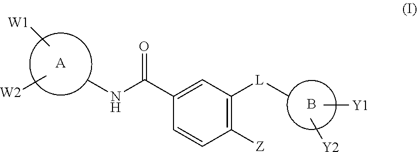

wherein
A is a ring selected from the group consisting of:

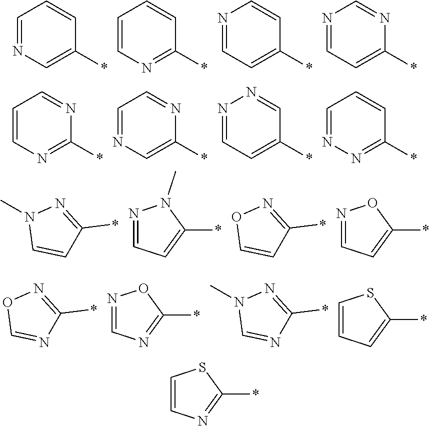

and phenyl, wherein [symbol] indicates a direct bond to NH;
W1 and W2 are substituents of ring A selected from the group consisting of hydrogen, halogen, (C₁-C₄)alkyl, (C₁-C₄)haloalkyl, (C₁-C₄)alkoxy, (C₁-C₄)haloalkoxy, R1R2N—(C₁-C₄)alkyl and (C₃-C₆)cycloalkyl;
Z is selected from the group consisting of (C₁-C₄)alkyl, (C₁-C₄)haloalkyl, (C₁-C₄)alkoxy, (C₁-C₄)haloalkoxy, (C₁-C₄)hydroxyalkyl and (C₃-C₆)cycloalkyl;
L is selected from the group consisting of

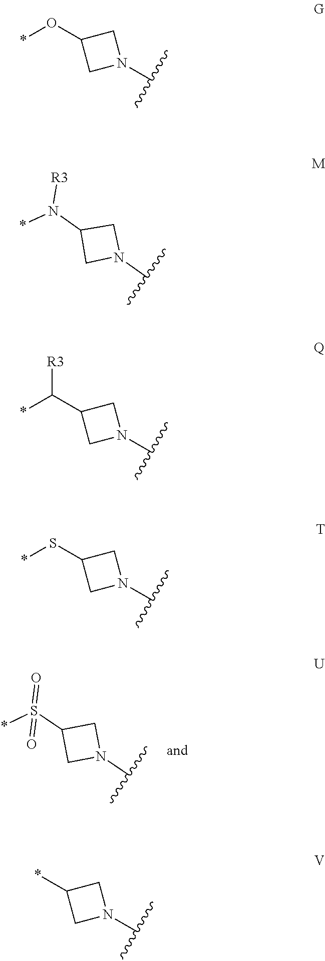

wherein [symbol] indicates a direct bond to the phenyl ring and

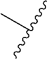

indicates a direct bond to B;
B is mono- or bi-cyclic heteroaryl ring;
Y1 and Y2 are substituents of ring B independently selected from the group consisting of hydrogen, (C₁-C₄)alkyl, (C₁-C₄)haloalkyl, (C₁-C₄)alkoxy, (C₁-C₄)alkoxy-(C₁-C₄)alkoxy, hydroxy-(C₁-C₄)alkoxy, hydroxy-(C₁-C₄)alkyl, (C₁-C₄)alkyl-heterocycloalkyl-(C₀-C₄)alkoxy, (C₁-C₄)haloalkoxy, halogen, cyano, cyano-(C₁-C₄)alkyl, cyano-(C₁-C₄)alkyl-heterocycloalkyl, CONR1R2, NHCOR1, NR1R2, R1R2N—(C₁-C₄)alkyl, heterocycloalkyl, (C₁-C₄)alkyl-heterocycloalkyl, heterocycloalkyl-(C₁-C₄)alkyl, (C₁-C₄)alkyl-heterocycloalkyl-carbonyl, (C₀-C₄)alkyl-heterocycloalkyl-carbonyl (C₁-C₄)alkyl-phenyl and monocyclic (C₁-C₄)alkyl-heteroaryl;
R1 and R2 are independently selected from the group consisting of hydrogen, (C₁-C₄)alkyl, (C₁-C₄)hydroxyalkyl, (C₁-C₄)alkoxy(C₁-C₄)alkyl, (C₁-C₄)alkylamino-(C₁-C₄)alkyl, di-(C₁-C₄)alkylamino-(C₁-C₄)alkyl, optionally substituted (C₃-C₆)cycloalkyl, optionally substituted heterocycloalkyl and optionally substituted heterocycloalkyl-(C₁-C₄)alkoxy, wherein optional substituents are one or more and are selected from (C₁-C₄)alkyl, (C₁-C₄)alkoxy, (C₁-C₄)haloalkyl and (C₁-C₄)haloalkoxy;
R3 is selected from hydrogen and (C₁-C₄)alkyl, preferably hydrogen and methyl;
or a stereoisomer, tautomer, solvate and pharmaceutically acceptable salt thereof.

All the listed meanings of each of the variable moieties A, B, L, Z, W1, W2, Y1, Y2, R1, R2 and R3 of the compound of formula (I) of the invention have to be intended as alternatives and may be combined with each other in embodiments which are included in the scope of the invention.

Preferred halogens in (C₁-C₄)haloalkyl and (C₁-C₄)haloalkoxy substituents are fluorine and chlorine, wherein fluorine is more preferred. Fluorine is also the preferred choice for W and Y substituents being halogen.

W1 and W2 are substituents of ring A which can be attached to A at any available position. W1 and W2 are preferably selected from the group consisting of H, F, CH₃, OCH₃, OCF₃, CF₃, C(CH₃)₃, CH₂CH₃, C(CH₃)₂CF₃, OCF₂H, CHF₂, CH₂CF₃, CH₂N(CH₃)₂ and cyclopropyl.

Z is preferably selected from the group consisting of CH₃, CF₂H, OCH₃, CH₂OH and cyclopropyl.

In one embodiment, L is selected from the group consisting of

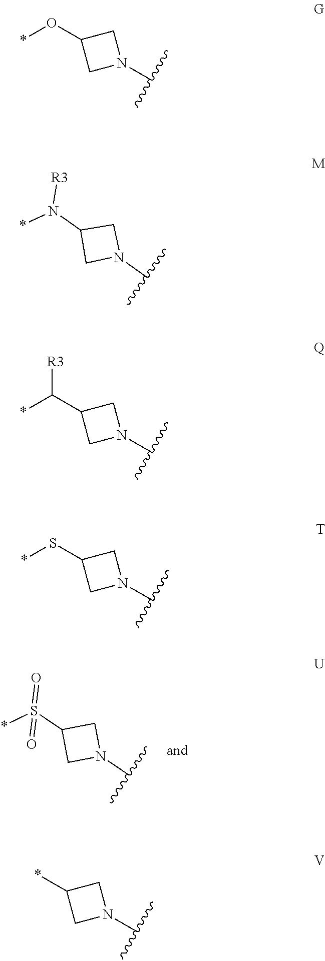

in a preferred embodiment, L is selected from the group consisting of

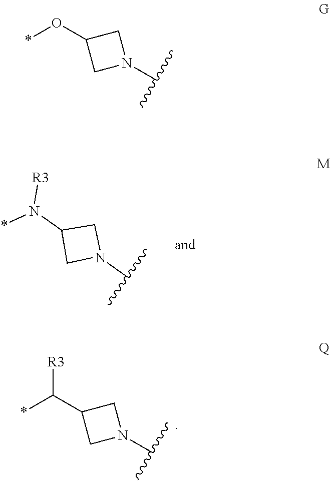

B is mono- or bi-cyclic heteroaryl ring, preferably selected from the group consisting of pyridinyl, pyrimidinyl, pyrazinyl, pyrazolo[1,5-a]pyrazinyl, 1H-pyrazolo[3,4-b]pyridinyl, pyrazolo[1,5-a]pyridinyl, pyrazolo[1,5-a]pyrimidinyl, imidazo[1,2-b]pyridazinyl, imidazo[1,2-a]pyrazinyl, imidazo[1,2-a]pyridinyl, thieno[3,2-d]pyrimidinyl, 1H-pyrrolo[2,3-b]pyridinyl and pyrazolyl. Particularly preferred B rings are pyridin-3-yl, pyrimidin-5-yl, pyrazinyl, pyrazolo[1,5-a]pyridin-3-yl, pyrazolo[1,5-a]pyrazin-3-yl, pyrazolo[1,5-a]pyrimidin-3-yl, imidazo[1,2-a]pyrazin-3-yl, 1H-pyrazolo[3,4-b]pyridin-5-yl and imidazo[1,2-b]pyridazin-3-yl.
Y1 is a substituent of ring B which can be attached to B at any available position. Y1 is preferably selected from the group consisting of hydrogen, (C₁-C₄)haloalkyl, (C₁-C₄)alkoxy, cyano, CONR1R2, NHCOR1, NR1R2, (C₁-C₄)alkyl-heterocycloalkyl-carbonyl and monocyclic (C₁-C₄)alkyl-heteroaryl; more preferably Y1 is selected from the group consisting of hydrogen, CONH₂, CF₂H, OCH₃, cyano, NHCOCH₃, NH₂, 4-methylpiperazine-1-carbonyl, 4-methylpiperazin-1-yl and 1-methyl-1H-pyrazol-4-yl.
Y2 is a substituent of ring B which can be attached to B at any available position. In preferred embodiments Y2 is hydrogen.
R1 and R2 are independently preferably selected from the group consisting of CH₂CH₂N(CH₃)₂, CH₂CH₂OCH₃, CH₃, CH₂CH₂OH, oxetan-3-yl and 3-hydroxycyclobutyl.
R3 is preferably hydrogen or methyl.

Optional substituents of R1 and/or R2 are one or more, preferably 1 to 3, and are selected from the group consisting of (C₁-C₄)alkyl, (C₁-C₄)alkoxy, (C₁-C₄)haloalkyl and (C₁-C₄)haloalkoxy, preferably selected from CH₃, OCH₃, OCF₃, CF₃, CF₂H, C(CH₃)₃, C(CH₃)₂CF₃ and OCF₂H.

Accordingly, in a preferred embodiment, the invention relates to a compound of formula (I) wherein
A is a ring selected from the group consisting of:

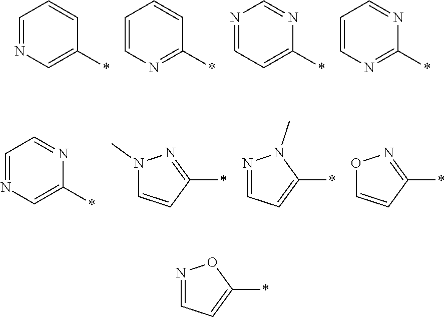

and phenyl;
W1 and W2 are selected from the group consisting of H, F, CH₃, OCH₃, OCF₃, CF₃, C(CH₃)₃, CH₂CH₃, C(CH₃)₂CF₃, OCF₂H, CHF₂, CH₂CF₃, CH₂N(CH₃)₂ and cyclopropyl;
Z is selected from the group consisting of CH₃, CF₂H, OCH₃, CH₂OH and cyclopropyl;
L is selected from the group consisting of

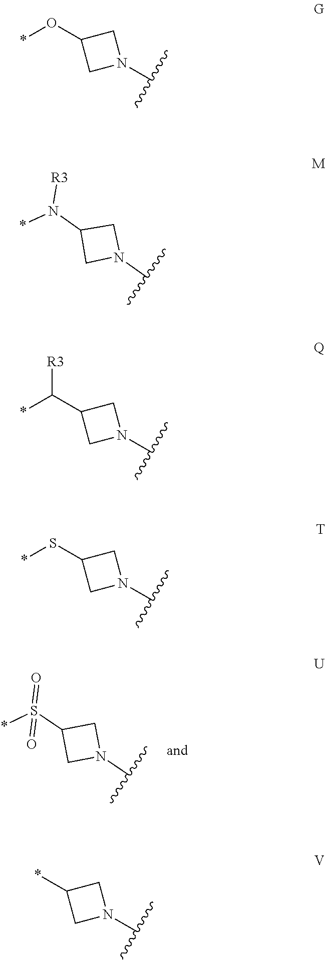

or L is selected from the group consisting of

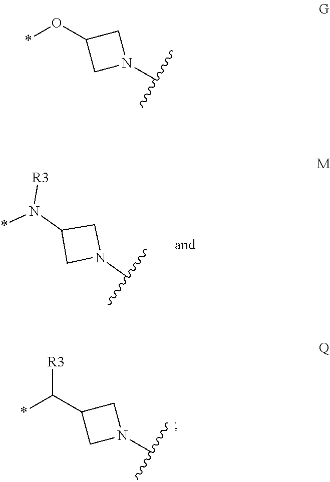

B is selected from the group consisting of pyridinyl, pyrimidinyl, pyrazinyl, pyrazolo[1,5-a]pyrazinyl, 1H-pyrazolo[3,4-b]pyridinyl, pyrazolo[1,5-a]pyridinyl, pyrazolo[1,5-a]pyrimidinyl, imidazo[1,2-b]pyridazinyl, imidazo[1,2-a]pyrazinyl, imidazo[1,2-a]pyridinyl, thieno[3,2-d]pyrimidinyl, 1H-pyrrolo[2,3-b]pyridinyl and pyrazolyl;
Y1 is selected from the group consisting of hydrogen, CONH₂, CF₂H, OCH₃, cyano, NHCOCH₃, NH₂, 4-methylpiperazine-1-carbonyl and 4-methylpiperazin-1-yl, and Y2 is hydrogen;
R1 and R2 are independently selected from the group consisting of CH₂CH₂N(CH₃)₂, CH₂CH₂OCH₃, CH₃, CH₂CH₂OH, oxetan-3-yl and 3-hydroxycyclobutyl;
R3 is hydrogen or methyl;
or a stereoisomer, tautomer, solvate and pharmaceutically acceptable salt thereof.

The present invention relates to a compound of formula (I), wherein L is selected from the group consisting of G, M, Q, T, U and V as defined above, which is also referred to as a compound of formula (Ic):

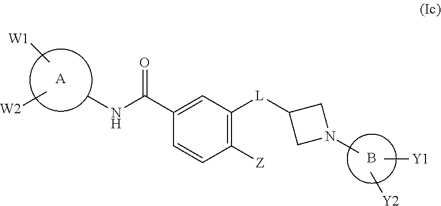

wherein L1 is selected from the group consisting of O, NR3, CHR3, S and SO₂, or is absent, L1 being preferably selected from O, NR3 and CHR3, and R3 being preferably hydrogen or methyl.

Further particularly preferred embodiments of the invention are the compounds of Formula (Ic) listed in Table 1 below, and pharmaceutically acceptable salts thereof. These compounds are particularly active on receptors DDR1 and DDR2, as shown in Table 2 further below.

TABLE 1
List of representative compounds of Formula (Ic)

| Example | | |
|---|---|---|
| N° | Structure | Chemical Name |
| 135 | 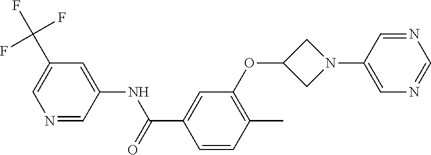 [SMILES: Cc1ccc(C(=O)Nc2cncc(C(F)(F)F)c2)cc1OC1CN(c2cncnc2)C1] | 4-methyl-3-((1-(pyrimidin- 5-yl)azetidin-3-yl)oxy)-N- (5-(trifluoromethyl)pyridin- 3-yl)benzamide |
| 136 | 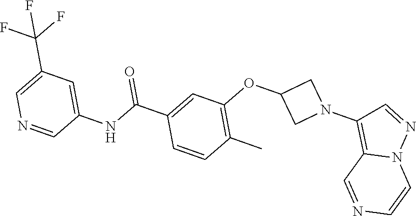 [SMILES: Cc1ccc(C(=O)Nc2cncc(C(F)(F)F)c2)cc1OC1CN(c2cnn3ccncc23)C1] | 4-methyl-3-((1- (pyrazolo[1,5-a]pyrazin-3- yl)azetidin-3-yl)oxy)-N-(5- (trifluoromethyl)pyridin-3- yl)benzamide |
| 137 | 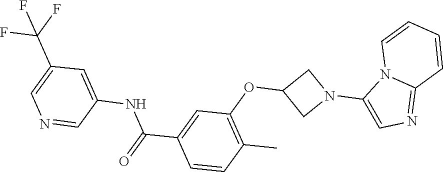 [SMILES: Cc1ccc(C(=O)Nc2cncc(C(F)(F)F)c2)cc1OC1CN(c2cnc3ccccn23)C1] | 3-((1-(imidazo[1,2- a]pyridin-3-yl)azetidin-3- yl)oxy)-4-methyl-N-(5- (trifluoromethyl)pyridin-3- yl)benzamide |
| 138 | 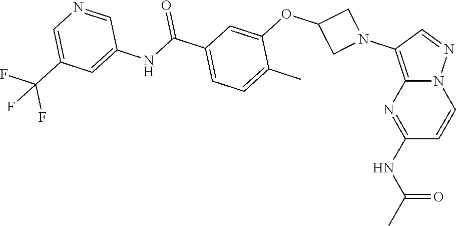 [SMILES: CC(=O)Nc1ccn2ncc(N3CC(Oc4cc(C(=O)Nc5cncc(C(F)(F)F)c5)ccc4C)C3)c2n1] | 3-((1-(5- acetamidopyrazolo[1,5- a]pyrimidin-3-yl)azetidin- 3-yl)oxy)-4-methyl-N-(5- (trifluoromethyl)pyridin-3- yl)benzamide |
| 139 | 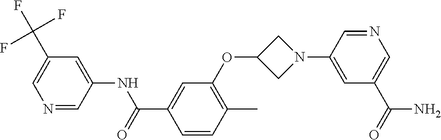 [SMILES: Cc1ccc(C(=O)Nc2cncc(C(F)(F)F)c2)cc1OC1CN(c2cncc(C(N)=O)c2)C1] | 5-(3-(2-methyl-5-((5- (trifluoromethyl)pyridin-3- yl)carbamoyl)phenoxy) azetidin-1-yl)nicotinamide |
| 141 | 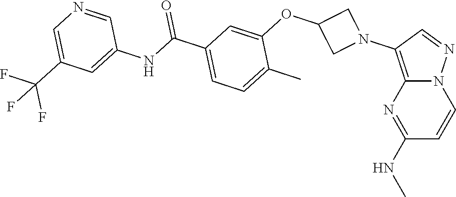 [SMILES: CNc1ccn2ncc(N3CC(Oc4cc(C(=O)Nc5cncc(C(F)(F)F)c5)ccc4C)C3)c2n1] | 4-methyl-3-((1-(5- (methylamino)pyrazolo[1,5- a]pyrimidin-3-yl)azetidin- 3-yl)oxy)-N-(5- (trifluoromethyl)pyridin-3- yl)benzamide |
| 142 | 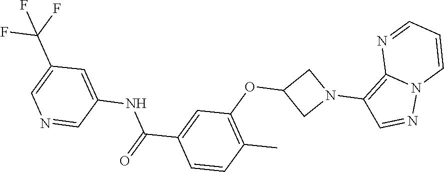 [SMILES: Cc1ccc(C(=O)Nc2cncc(C(F)(F)F)c2)cc1OC1CN(c2cnn3cccnc23)C1] | 4-methyl-3-((1- (pyrazolo[1,5-a]pyrimidin- 3-yl)azetidin-3-yl)oxy)-N- (5-(trifluoromethyl) pyridin-3-yl)benzamide |
| 143 | 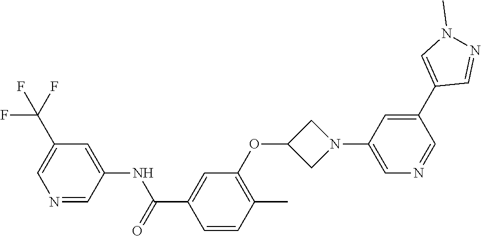 [SMILES: Cc1ccc(C(=O)Nc2cncc(C(F)(F)F)c2)cc1OC1CN(c2cncc(-c3cnn(C)c3)c2)C1] | 4-methyl-3-((1-(5-(1- methyl-1H-pyrazol-4- yl)pyridin-3-yl)azetidin-3- yl)oxy)-N-(5- (trifluoromethyl)pyridin-3- yl)benzamide |
| 145 | 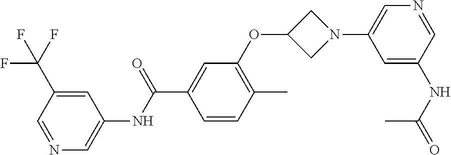 [SMILES: CC(=O)Nc1cncc(N2CC(Oc3cc(C(=O)Nc4cncc(C(F)(F)F)c4)ccc3C)C2)c1] | 3-((1-(5-acetamidopyridin- 3-yl)azetidin-3-yl)oxy)-4- methyl-N-(5- (trifluoromethyl)pyridin-3- yl)benzamide |
| 146 | 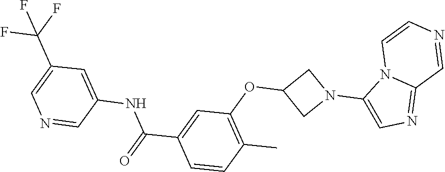 [SMILES: Cc1ccc(C(=O)Nc2cncc(C(F)(F)F)c2)cc1OC1CN(c2cnc3cnccn23)C1] | 3-((1-(imidazo[1,2- a]pyrazin-3-yl)azetidin-3- yl)oxy)-4-methyl-N-(5- (trifluoromethyl)pyridin-3- yl)benzamide |
| 152 | 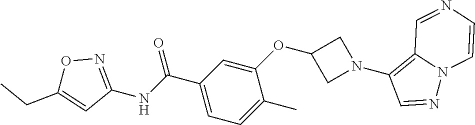 [SMILES: CCc1cc(NC(=O)c2ccc(C)c(OC3CN(c4cnn5ccncc45)C3)c2)no1] | N-(5-ethylisoxazol-3-yl)-4- methyl-3-((1-(pyrazolo[1,5- a]pyrazin-3-yl)azetidin-3- yl)oxy)benzamide |
| 153 | 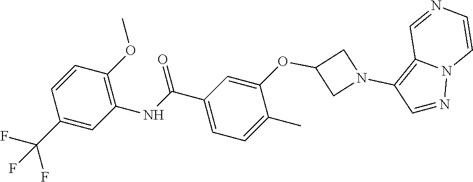 [SMILES: COc1ccc(C(F)(F)F)cc1NC(=O)c1ccc(C)c(OC2CN(c3cnn4ccncc34)C2)c1] | N-(2-methoxy-5- (trifluoromethyl)phenyl)-4- methyl-3-((1-(pyrazolo[1,5- a]pyrazin-3-yl)azetidin-3- yl)oxy)benzamide |
| 154 | 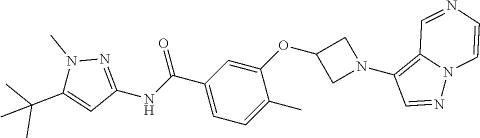 [SMILES: Cc1ccc(C(=O)Nc2cc(C(C)(C)C)n(C)n2)cc1OC1CN(c2cnn3ccncc23)C1] | N-(5-(tert-butyl)-1-methyl- 1H-pyrazol-3-yl)-4-methyl- 3-((1-(pyrazolo[1,5- a]pyrazin-3-yl)azetidin-3- yl)oxy)benzamide |
| 155 | 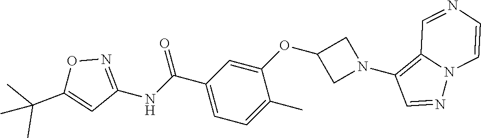 [SMILES: Cc1ccc(C(=O)Nc2cc(C(C)(C)C)on2)cc1OC1CN(c2cnn3ccncc23)C1] | N-(5-(tert-butyl)isoxazol-3- yl)-4-methyl-3-((1- (pyrazolo[1,5-a]pyrazin-3- yl)azetidin-3- yl)oxy)benzamide |
| 157 | 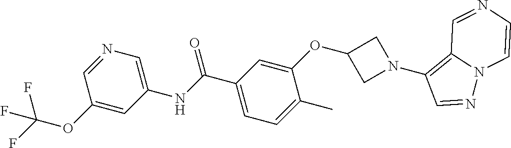 [SMILES: Cc1ccc(C(=O)Nc2cncc(OC(F)(F)F)c2)cc1OC1CN(c2cnn3ccncc23)C1] | 4-methyl-3-((1- (pyrazolo[1,5-a]pyrazin-3- yl)azetidin-3-yl)oxy)-N-(5- (trifluoromethoxy)pyridin- 3-yl)benzamide |
| 158 | 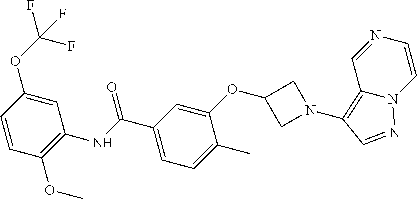 [SMILES: COc1ccc(OC(F)(F)F)cc1NC(=O)c1ccc(C)c(OC2CN(c3cnn4ccncc34)C2)c1] | N-(2-methoxy-5- (trifluoromethoxy)phenyl)- 4-methyl-3-((1- (pyrazolo[1,5-a]pyrazin-3- yl)azetidin-3- yl)oxy)benzamide |
| 159 | 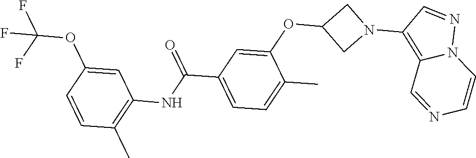 [SMILES: Cc1ccc(OC(F)(F)F)cc1NC(=O)c1ccc(C)c(OC2CN(c3cnn4ccncc34)C2)c1] | 4-methyl-N-(2-methyl-5- (trifluoromethoxy)phenyl)- 3-((1-(pyrazolo[1,5- a]pyrazin-3-yl)azetidin-3- yl)oxy)benzamide |
| 160 | 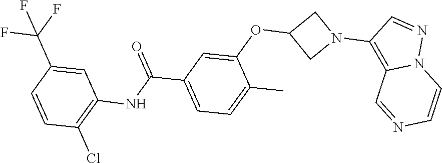 | N-(2-chloro-5- (trifluoromethyl)phenyl)-4- methyl-3-((1-(pyrazolo[1,5- a]pyrazin-3-yl)azetidin-3- yl)oxy)benzamide |
| 161 | 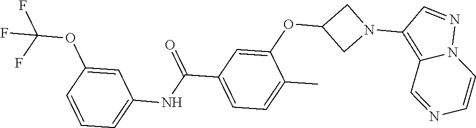 [SMILES: Cc1ccc(C(=O)Nc2cccc(OC(F)(F)F)c2)cc1OC1CN(c2cnn3ccncc23)C1] | 4-methyl-3-((1- (pyrazolo[1,5-a]pyrazin-3- yl)azetidin-3-yl)oxy)-N-(3- (trifluoromethoxy)phenyl) benzamide |
| 162 | 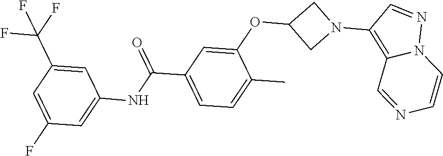 [SMILES: Cc1ccc(C(=O)Nc2cc(F)cc(C(F)(F)F)c2)cc1OC1CN(c2cnn3ccncc23)C1] | N-(3-fluoro-5- (trifluoromethyl)phenyl)-4- methyl-3-((1-(pyrazolo[1,5- a]pyrazin-3-yl)azetidin-3- yl)oxy)benzamide |
| 163 | 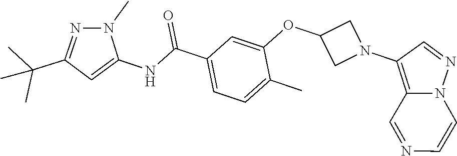 [SMILES: Cc1ccc(C(=O)Nc2cc(C(C)(C)C)nn2C)cc1OC1CN(c2cnn3ccncc23)C1] | N-(3-(tert-butyl)-1-methyl- 1H-pyrazol-5-yl)-4-methyl- 3-((1-(pyrazolo[1,5- a]pyrazin-3-yl)azetidin-3- yl)oxy)benzamide |
| 164 | 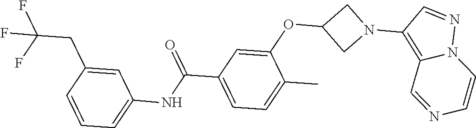 [SMILES: Cc1ccc(C(=O)Nc2cccc(CC(F)(F)F)c2)cc1OC1CN(c2cnn3ccncc23)C1] | 4-methyl-3-((1- (pyrazolo[1,5-a]pyrazin-3- yl)azetidin-3-yl)oxy)-N-(3- (2,2,2- trifluoroethyl)phenyl) benzamide |
| 165 | 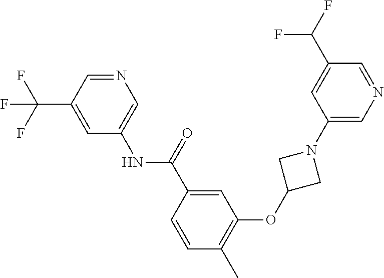 [SMILES: Cc1ccc(C(=O)Nc2cncc(C(F)(F)F)c2)cc1OC1CN(c2cncc(C(F)F)c2)C1] | 3-((1-(5- (difluoromethyl)pyridin-3- yl)azetidin-3-yl)oxy)-4- methyl-N-(5- (trifluoromethyl)pyridin-3- yl)benzamide |
| 166 | 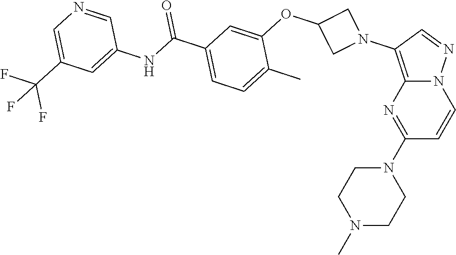 [SMILES: Cc1ccc(C(=O)Nc2cncc(C(F)(F)F)c2)cc1OC1CN(c2cnn3ccc(N4CCN(C)CC4)nc23)C1] | 4-methyl-3-((1-(5-(4- methylpiperazin-1- yl)pyrazolo[1,5- a]pyrimidin-3-yl)azetidin- 3-yl)oxy)-N-(5- (trifluoromethyl)pyridin-3- yl)benzamide |
| 167 | 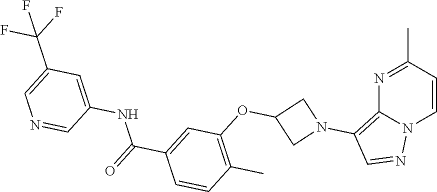 [SMILES: Cc1ccn2ncc(N3CC(Oc4cc(C(=O)Nc5cncc(C(F)(F)F)c5)ccc4C)C3)c2n1] | 4-methyl-3-((1-(5- methylpyrazolo[1,5- a]pyrimidin-3-yl)azetidin- 3-yl)oxy)-N-(5- (trifluoromethyl)pyridin-3- yl)benzamide |
| 168 | 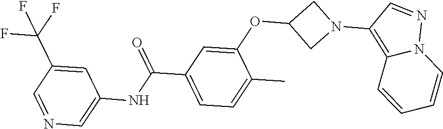 [SMILES: Cc1ccc(C(=O)Nc2cncc(C(F)(F)F)c2)cc1OC1CN(c2cnn3ccccc23)C1] | 4-methyl-3-((1- (pyrazolo[1,5-a]pyridin-3- yl)azetidin-3-yl)oxy)-N-(5- (trifluoromethyl)pyridin-3- yl)benzamide |
| 169 | 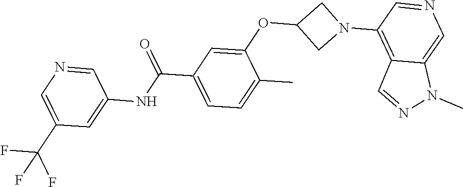 [SMILES: Cc1ccc(C(=O)Nc2cncc(C(F)(F)F)c2)cc1OC1CN(c2cncc3c2cnn3C)C1] | 4-methyl-3-((1-(1-methyl- 1H-pyrazolo[3,4-c]pyridin- 4-yl)azetidin-3-yl)oxy)-N- (5-(trifluoromethyl)pyridin- 3-yl)benzamide |
| 170 | 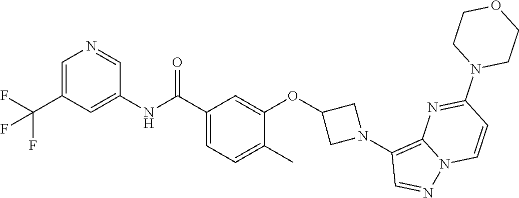 [SMILES: Cc1ccc(C(=O)Nc2cncc(C(F)(F)F)c2)cc1OC1CN(c2cnn3ccc(N4CCOCC4)nc23)C1] | 4-methyl-3-((1-(5- morpholinopyrazolo[1,5- a]pyrimidin-3-yl)azetidin- 3-yl)oxy)-N-(5- (trifluoromethyl)pyridin-3- yl)benzamide |
| 173 | 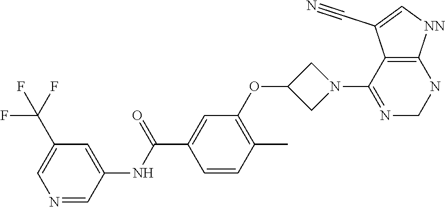 | 3-((1-(5-cyano-7H- pyrrolo[2,3-d]pyrimidin-4- yl)azetidin-3-yl)oxy)-4- methyl-N-(5- (trifluoromethyl)pyridin-3- yl)benzamide |
| 174 | 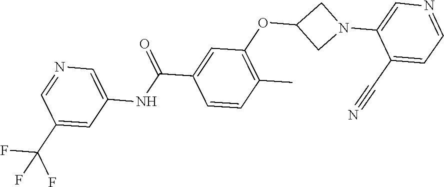 [SMILES: Cc1ccc(C(=O)Nc2cncc(C(F)(F)F)c2)cc1OC1CN(c2cnccc2C#N)C1] | 3-((1-(4-cyanopyridin-3- yl)azetidin-3-yl)oxy)-4- methyl-N-(5- (trifluoromethyl)pyridin-3- yl)benzamide |
| 180 | 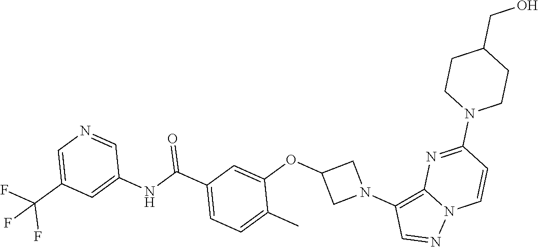 [SMILES: Cc1ccc(C(=O)Nc2cncc(C(F)(F)F)c2)cc1OC1CN(c2cnn3ccc(N4CCC(CO)CC4)nc23)C1] | 3-((1-(5-(4- (hydroxymethyl)piperidin- 1-yl)pyrazolo[1,5- a]pyrimidin-3-yl)azetidin- 3-yl)oxy)-4-methyl-N-(5- (trifluoromethyl)pyridin-3- yl)benzamide |
| 182 | 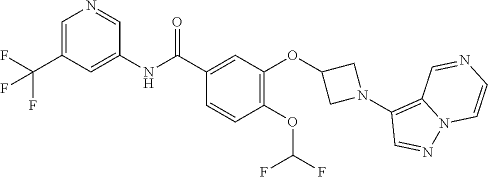 [SMILES: O=C(Nc1cncc(C(F)(F)F)c1)c1ccc(OC(F)F)c(OC2CN(c3cnn4ccncc34)C2)c1] | 4-(difluoromethoxy)-3-((1- (pyrazolo[1,5-a]pyrazin-3- yl)azetidin-3-yl)oxy)-N-(5- (trifluoromethyl)pyridin-3- yl)benzamide |
| 183 | 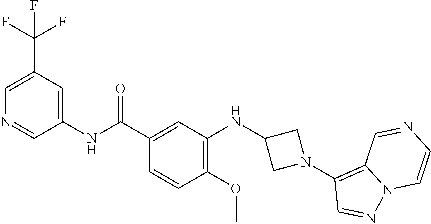 [SMILES: COc1ccc(C(=O)Nc2cncc(C(F)(F)F)c2)cc1NC1CN(c2cnn3ccncc23)C1] | 4-methoxy-3-((1- (pyrazolo[1,5-a]pyrazin-3- yl)azetidin-3-yl)amino)-N- (5-(trifluoromethyl)pyridin- 3-yl)benzamide |
| 134 | 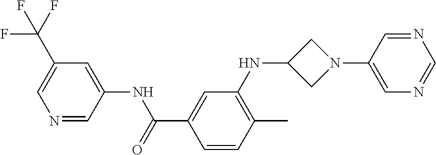 [SMILES: Cc1ccc(C(=O)Nc2cncc(C(F)(F)F)c2)cc1NC1CN(c2cncnc2)C1] | 4-methyl-3-((1-(pyrimidin- 5-yl)azetidin-3-yl)amino)- N-(5- (trifluoromethyl)pyridin-3- yl)benzamide |
| 140 | 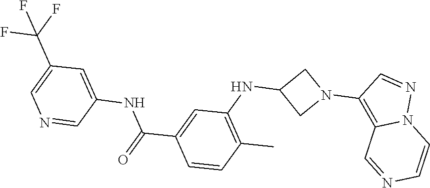 [SMILES: Cc1ccc(C(=O)Nc2cncc(C(F)(F)F)c2)cc1NC1CN(c2cnn3ccncc23)C1] | 4-methyl-3-((1- (pyrazolo[1,5-a]pyrazin-3- yl)azetidin-3-yl)amino)-N- (5-(trifluoromethyl)pyridin- 3-yl)benzamide |
| 144 | 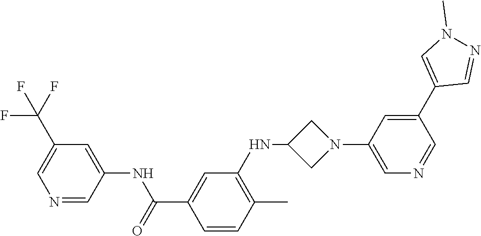 [SMILES: Cc1ccc(C(=O)Nc2cncc(C(F)(F)F)c2)cc1NC1CN(c2cncc(-c3cnn(C)c3)c2)C1] | 4-methyl-3-((1-(5-(1- methyl-1H-pyrazol-4- yl)pyridin-3-yl)azetidin-3- yl)amino)-N-(5- (trifluoromethyl)pyridin-3- yl)benzamide |
| 147 | 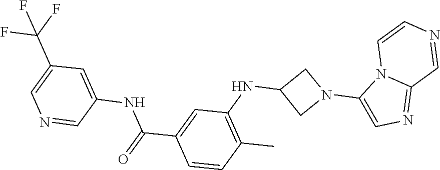 [SMILES: Cc1ccc(C(=O)Nc2cncc(C(F)(F)F)c2)cc1NC1CN(c2cnc3cnccn23)C1] | 3-((1-(imidazo[1,2- a]pyrazin-3-yl)azetidin-3- yl)amino)-4-methyl-N-(5- (trifluoromethyl)pyridin-3- yl)benzamide |
| 148 | 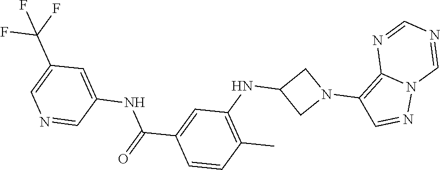 [SMILES: Cc1ccc(C(=O)Nc2cncc(C(F)(F)F)c2)cc1NC1CN(c2cnn3cncnc23)C1] | 4-methyl-3-((1- (pyrazolo[1,5-a]pyrimidin- 3-yl)azetidin-3-yl)amino)- N-(5- (trifluoromethyl)pyridin-3- yl)benzamide |
| 149 | 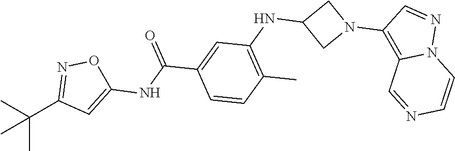 [SMILES: Cc1ccc(C(=O)Nc2cc(C(C)(C)C)no2)cc1NC1CN(c2cnn3ccncc23)C1] | N-(3-(tert-butyl)isoxazol-5- yl)-4-methyl-3-((1- (pyrazolo[1,5-a]pyrazin-3- yl)azetidin-3- yl)amino)benzamide |
| 150 | 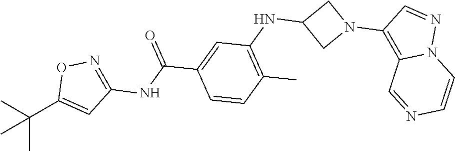 | N-(5-(tert-butyl)isoxazol-3- yl)-4-methyl-3-((1- (pyrazolo[1,5-a]pyrazin-3- yl)azetidin-3- yl)amino)benzamide |
| 156 | 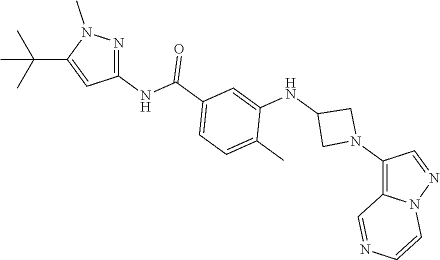 [SMILES: Cc1ccc(C(=O)Nc2cc(C(C)(C)C)n(C)n2)cc1NC1CN(c2cnn3ccncc23)C1] | N-(5-(tert-butyl)-1-methyl- 1H-pyrazol-3-yl)-4-methyl- 3-((1-(pyrazolo[1,5- a]pyrazin-3-yl)azetidin-3- yl)amino)benzamide |
| 181 | 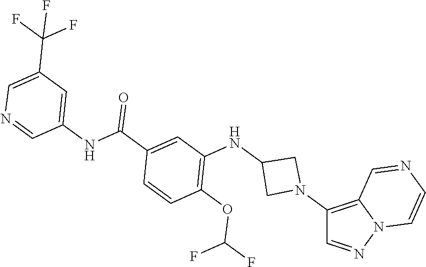 | 4-(difluoromethoxy)-3-((1- (pyrazolo[1,5-a]pyrazin-3- yl)azetidin-3-yl)amino)-N- (5-(trifluoromethyl)pyridin- 3-yl)benzamide |
| 151 | 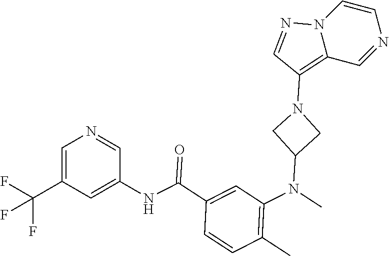 | 4-methyl-3-(methyl(1- (pyrazolo[1,5-a]pyrazin-3- yl)azetidin-3-yl)amino)-N- (5-(trifluoromethyl)pyridin- 3-yl)benzamide |
| 171 | 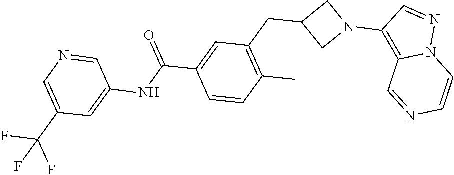 | 4-methyl-3-((1- (pyrazolo[1,5-a]pyrazin-3- yl)azetidin-3-yl)methyl)-N- (5-(trifluoromethyl)pyridin- 3-yl)benzamide |
| 172 | 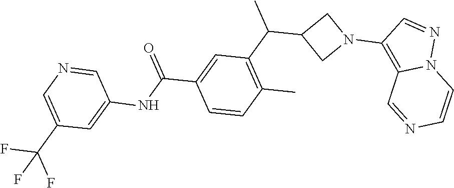 | 4-methyl-3-(1-(1- (pyrazolo[1,5-a]pyrazin-3- yl)azetidin-3-yl)ethyl)-N- (5-(trifluoromethyl)pyridin- 3-yl)benzamide |
| 175 | 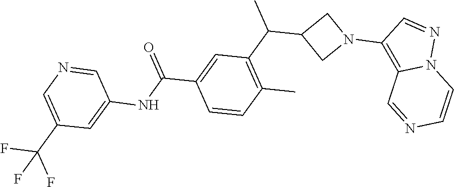 | 4-methyl-3-(1-(1- (pyrazolo[1,5-a]pyrazin-3- yl)azetidin-3-yl)ethyl)-N- (5-(trifluoromethyl)pyridin- 3-yl)benzamide 1^{st} eluted enantiomer |
| 176 | 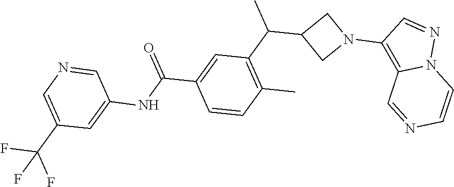 | 4-methyl-3-(1-(1- (pyrazolo[1,5-a]pyrazin-3- yl)azetidin-3-yl)ethyl)-N- (5-(trifluoromethyl)pyridin- 3-yl)benzamide 2^{nd} eluted enantiomer |
| 177 | 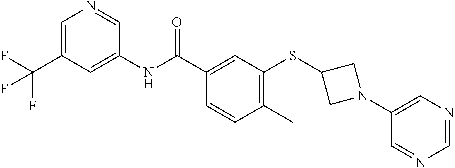 [SMILES: Cc1ccc(C(=O)Nc2cncc(C(F)(F)F)c2)cc1SC1CN(c2cncnc2)C1] | 4-methyl-3-((1-(pyrimidin- 5-yl)azetidin-3-yl)thio)-N- (5-(trifluoromethyl)pyridin- 3-yl)benzamide |
| 178 | 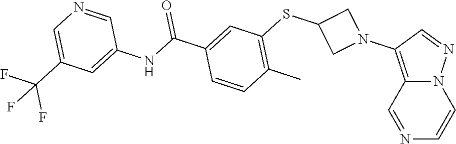 | 4-methyl-3-((1- (pyrazolo[1,5-a]pyrazin-3- yl)azetidin-3-yl)thio)-N-(5- (trifluoromethyl)pyridin-3- yl)benzamide |
| 179 | 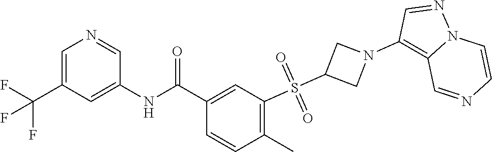 | 4-methyl-3-((1- (pyrazolo[1,5-a]pyrazin-3- yl)azetidin-3-yl)sulfonyl)- N-(5- (trifluoromethyl)pyridin-3- yl)benzamide |
| 184 | 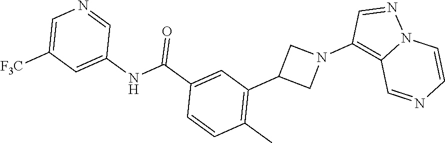 | 4-methyl-3-(1- (pyrazolo[1,5-a]pyrazin-3- yl)azetidin-3-yl)-N-(5- (trifluoromethyl)pyridin-3- yl)benzamide |

The compounds of the invention, including all the compounds here above listed, can be prepared from readily available starting materials using the following general methods and procedures or by using slightly modified processes readily available to those of ordinary skill in the art. Although a particular embodiment of the present invention may be shown or described herein, those skilled in the art will recognize that all embodiments or aspects of the present invention can be obtained using the methods described herein or by using other known methods, reagents and starting materials. When typical or preferred process conditions (i.e. reaction temperatures, times, mole ratios of reactants, solvents, pressures, etc.) are given, other process conditions can also be used unless otherwise stated. While the optimal reaction conditions may vary depending on the particular reactants or solvent used, such conditions can be readily determined by those skilled in the art by routine optimization procedures. Thus, processes described below should not be viewed as limiting the scope of the synthetic methods available for the preparation of the compounds of the invention.

In some cases, generally known protective groups (PG) may be employed when needed to mask or protect sensitive or reactive moieties, in accordance to general principles of chemistry (Protective group in organic syntheses, 3rd ed. T. W. Greene, P. G. M. Wuts).

Compounds of formula (I) may be prepared as described hereinafter, in Schemes 1 to 7, wherein at least one non-limiting synthetic route is provided for the preparation of the exemplified compounds (i.e. Examples).

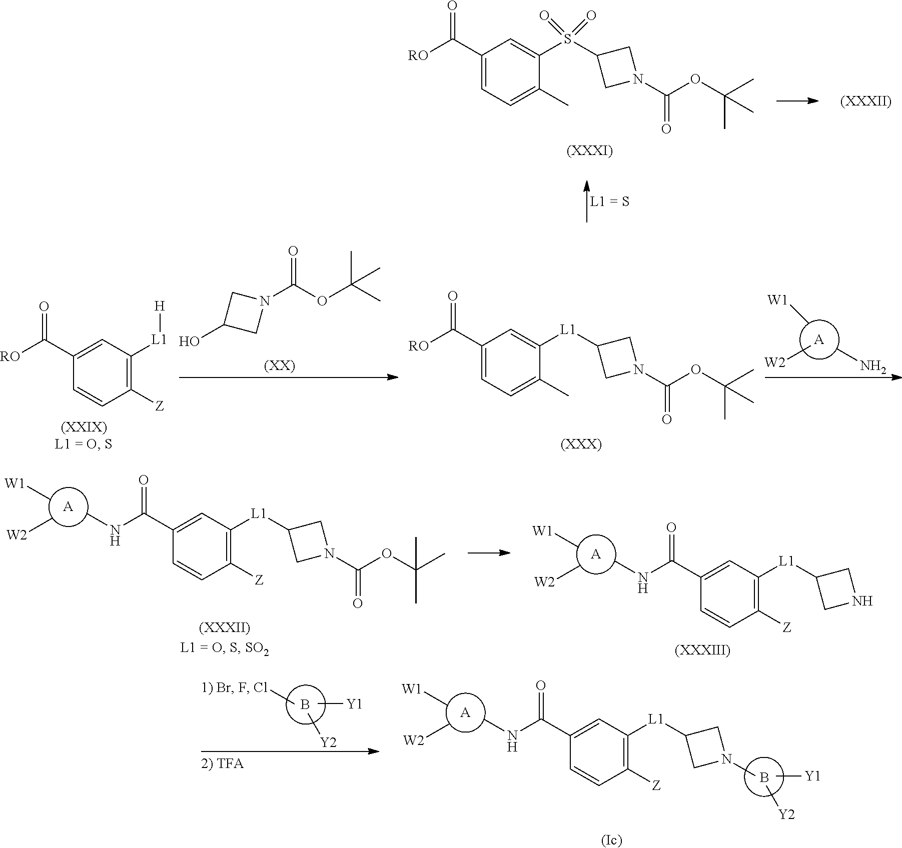

Compounds of formula (Ic), wherein L1 is selected from the group consisting of O, S and SO₂, and which are compounds of formula (I) wherein L is selected from the group consisting of:

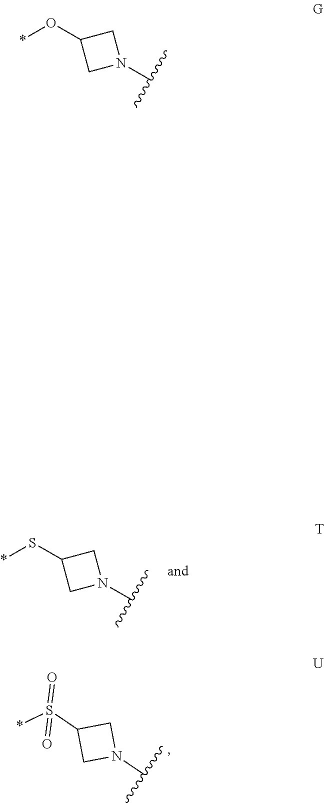 [SMILES: *OC1CN(*)C1 ; *SC1CN(*)C1 ; *N1CC(S(*)(=O)=O)C1]

can be prepared as depicted in Scheme 1 by starting from corresponding compounds of formula (XXIX) and formula (XX).

As described in Scheme 1, compounds of formula (XXIX), wherein Z is as defined above and R is (C₁-C₄)alkyl, preferably ethyl, and wherein L1 is O or S, can be reacted with tert-butyl 3-hydroxyazetidine-1-carboxylate (XX), by performing a Mitsunobu reaction, in the presence of a coupling reagent, as Tsunoda reagent, in an appropriate solvent, like toluene, at high temperature (around 70° C.) overnight. Compounds of formula (XXX) can be reacted by ox-red reaction in the presence of oxone, and in an appropriate solvent, such as ethanol. Compounds of formula (XXXIII) can be prepared by reacting compounds of formula (XXX) or (XXXI) with an heteroaryl amine by means of a transamidation reaction in the presence of a strong base, such as BuLi, LiHMDS or LDA, in an appropriate solvent, such as THF, followed by the removal of the protecting group in acidic conditions, using TFA or HCl in 4-dioxane. Compounds of formula (XXXIII) can be converted into compounds of formula (Ic) by means of a Buchwald coupling reaction, using an appropriate palladium catalyst such as Pd₂(dppf)Cl₂, Buchwald 3rd generation catalysts or any other palladium source/phosphine-based ligand, at high temperature (around 100° C.) for few hours, in the presence of a base, such as Cs₂CO₃, NaOtBu or LiHMDS, in the appropriate solvent, such as 4-dioxane or DMA.

Alternatively, compounds of formula (XXXIII) can be converted into compounds of formula (Ic) through a nucleophilic aromatic substitution (S_{N}Ar) using the suitable commercially available fluoro-heteroaryl or chloro-heteroaryl compounds in the presence of a suitable base, such as DIPEA, and the appropriate solvent, such as acetonitrile.

If a protecting group is present on Y1 and/or Y2, compounds of formula (Ic) can be obtained for instance using TFA, in the appropriate solvent, such as DCM (optional step indicated as “2) TFA” in Scheme 1).

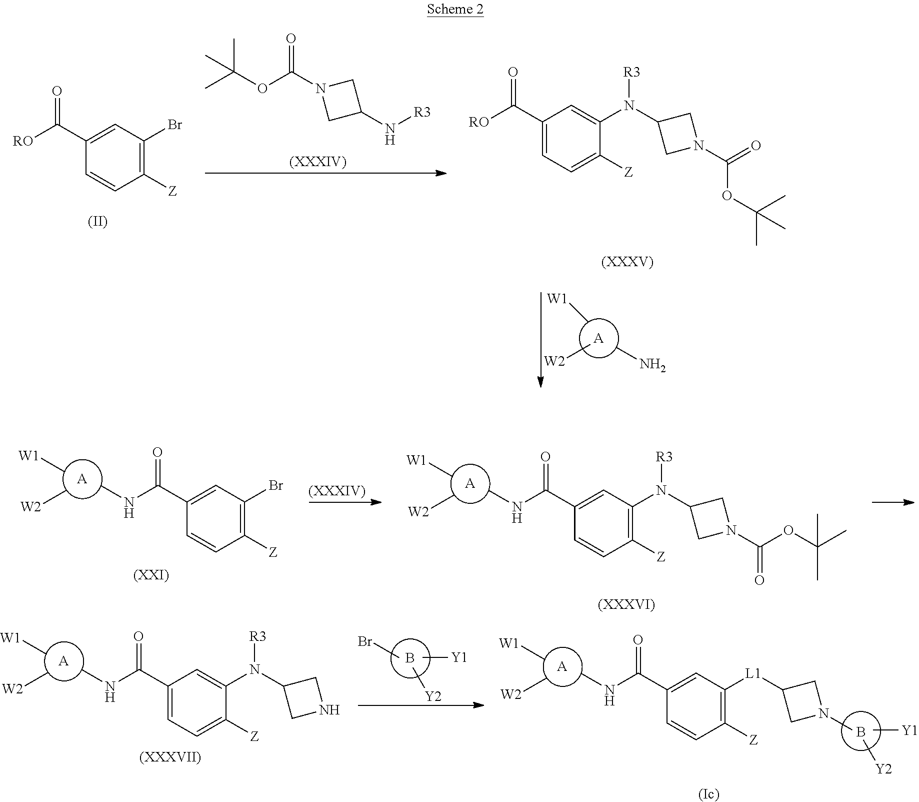

Compounds of formula (Ic), wherein L1 is NR3, which are compounds of formula (I) wherein L is

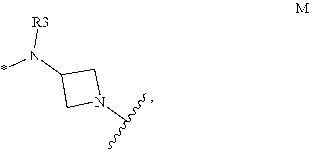

may be prepared as depicted in Scheme 2 by starting from compounds of formula (II) or (XXI). Compounds of formula (II) or compounds of formula (XXI), wherein Z is as defined above and R is (C₁-C₄)alkyl, preferably ethyl, can be reacted with compounds of formula (XXXIV), for instance tert-butyl 3-aminoazetidine-1-carboxylate or tert-butyl 3-(methylamino)azetidine-1-carboxylate, by performing a Buchwald coupling reaction, using an appropriate palladium catalyst, such as Buchwald 3rd generation catalysts or any other palladium source/phosphine-based ligand, at high temperature (around 100° C.) for few hours, in the presence of a base, such as Cs₂CO₃, NaOtBu or LiHMDS, in the appropriate solvent, such as 4-dioxane or DMA, to obtain a compound of formula (XXXV) or a compound of formula (XXXVI), respectively.

Compounds of formula (XXXVI) may also be prepared by reacting compounds of formula (XXXV) with an heteroaryl amine by means of a transamidation reaction in the presence of a strong base such as BuLi, LiHMDS or LDA in an appropriate solvent, such as THF. Compounds of formula (XXXVII) may then be prepared from compounds of formula (XXXVI) by the removal of the Boc protecting group in acidic conditions, using TFA or HCl in 1,4-dioxane. Compounds of formula (XXXVII) can then be converted into compounds of formula (Ic) by means of a Buchwald coupling reaction, using an appropriate palladium catalyst such as Pd₂(dppf)Cl₂, Buchwald 3rd generation catalysts or any other palladium source/phosphine-based ligand, at high temperature (around 100° C.) for few hours, in the presence of a base, such as Cs₂CO₃, NaOtBu or LiHMDS, in the appropriate solvent, such as 4-dioxane or DMA.

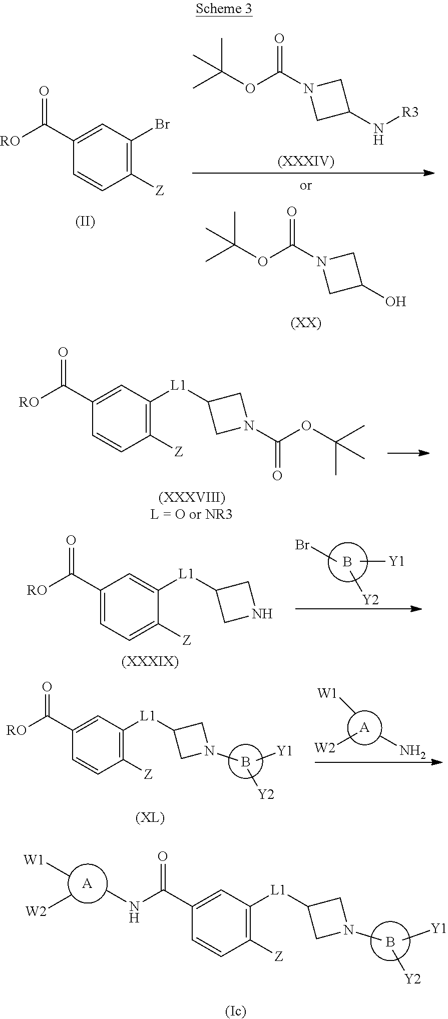

Otherwise, as described in Scheme 3, compounds of formula (II), wherein Z is as defined above and R is (C₁-C₄)alkyl, preferably ethyl, can be reacted with tert-butyl 3-hydroxyazetidine-1-carboxylate (XX) or compounds of formula (XXXIV) to obtain compounds of formula (XXXVIII) by performing a Buchwald coupling reaction, using an appropriate palladium catalyst such as Buchwald 3rd generation catalysts or any other palladium source/phosphine-based ligand, at high temperature (around 100° C.) for few hours, in the presence of a base, such as Cs₂CO₃, NaOtBu or LiHMDS, in the appropriate solvent, such as 4-dioxane or DMA. Compounds of formula (XXXIX) can be prepared by removal of the Boc protecting group in acidic conditions, using TFA or HCl in 4-dioxane. Compounds of formula (XL) can then be obtained by performing a Buchwald coupling reaction, using an appropriate palladium catalyst, such as Buchwald 3rd generation catalysts or any other palladium source/phosphine-based ligand, at high temperature (around 100° C.) for few hours, in the presence of a base, such as Cs₂CO₃, NaOtBu or LiHMDS, in the appropriate solvent, such as 4-dioxane or DMA. Compounds of formula (Ic), wherein L1 is O or NR3, may be prepared by reacting compounds of formula (XL) with an heteroaryl amine by means of a transamidation reaction in the presence of a strong base, such as BuLi, LiHMDS or LDA, in an appropriate solvent, such as THF. Alternatively, amidation may be performed using common coupling reagents such as HATU, in the presence of an organic base, such as DIPEA, in the appropriate solvent, such as DMF, to give compounds of formula (Ic).

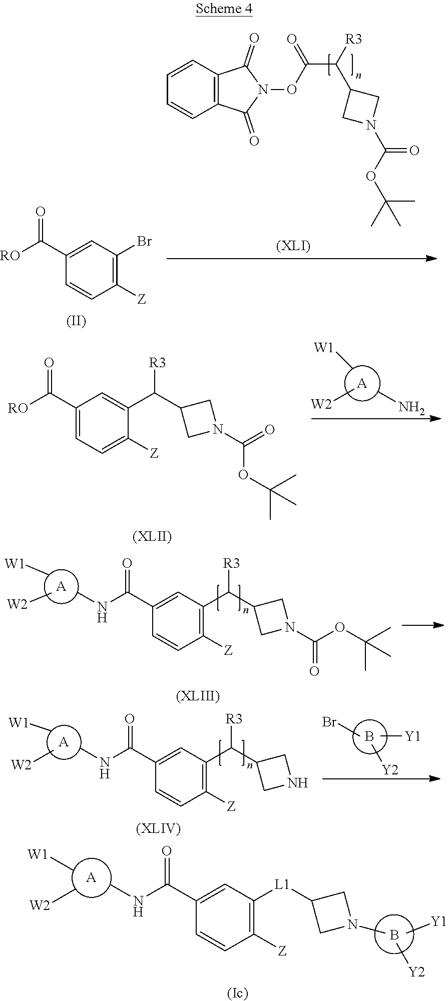

Compounds of formula (Ic), wherein L1 is CHR3 or is absent, and which are compounds of formula (I) wherein L is

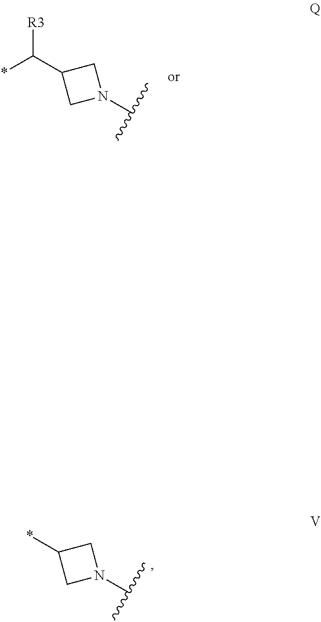

can be prepared as depicted in Scheme 4 by starting from corresponding compounds of formula (XLI) and (II). As described in Scheme 4, compounds of formula (II), wherein Z is as defined above and R is (C₁-C₄)alkyl, preferably ethyl, can be reacted with compounds of formula (XLI), wherein R3 is as defined above and n is 0 or 1, to obtain compounds of formula (XLII) by means of an electrochemical reaction in the presence of dibromonickel, and in presence of a salt, such as silver nitrate, in an appropriate solvent, such as NMP. Compounds of formula (XLIV) can be prepared by reacting compounds of formula (XLII) with an heteroaryl amine by means of a transamidation reaction in the presence of a strong base, such as BuLi, LiHMDS or LDA, in an appropriate solvent, such as THF, followed by the removal of the protecting group in acidic conditions, using TFA or HCl in 4-dioxane. Compounds of formula (Ic), wherein L1 is CHR3, can then be obtained by means of a Buchwald coupling reaction, using an appropriate palladium catalyst, such as Pd₂(dppf)Cl₂, Buchwald 3rd generation catalysts or any other palladium source/phosphine-based ligand, at high temperature (around 100° C.) for few hours, in the presence of a base, such as Cs₂CO₃, NaOtBu or LiHMDS, in the appropriate solvent, such as 4-dioxane or DMA.

Compounds of formula (Ic), wherein L1 is CHR3 and R3 is not hydrogen, comprise a stereocenter, which is the carbon atom bearing R3. These compounds are obtained as racemic mixtures. Separation of the racemic mixture may be achieved by chiral resolution methods such as chiral purification. Both enantiomers of compounds of formula (Ic) are included in the scope of the present invention.

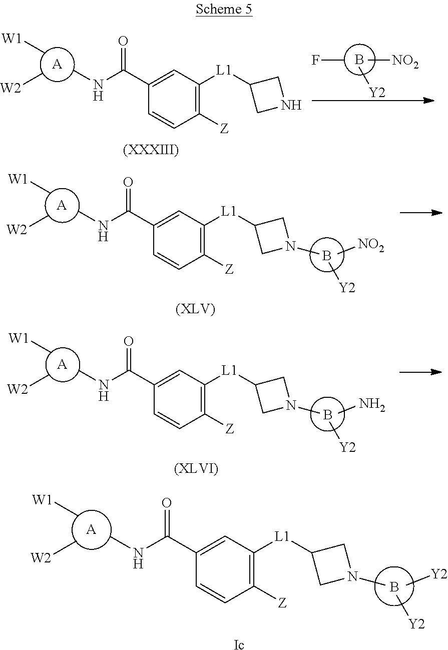

In a different approach, compounds of formula (XLV) can be obtained as depicted in Scheme 5 by starting from compounds of formula (XXXIII) and performing a Buchwald coupling reaction, with the appropriate palladium catalyst, such as Pd₂(dppf)Cl₂, Buchwald 3rd generation catalysts or any other palladium source/phosphine-based ligand, at high temperature (around 100° C.) for few hours, in the presence of a base, such as Cs₂CO₃, NaOtBu or LiHMDS, in the appropriate solvent, such as 4-dioxane or DMA. Compounds of formula (XLVI) can be obtained by reduction of the nitro group to amine group under hydrogen atmosphere and in the presence of a suitable catalyst, such as Pd/C, in a suitable solvent, such as EtOH or EtOAc. Compounds of formula (Ic) can be prepared by acylation of compounds of formula (XLVI) with the suitable commercially available acyl chloride, i.e. R1COCl, or anhydride, in the presence of an appropriate base, such as pyridine or TEA.

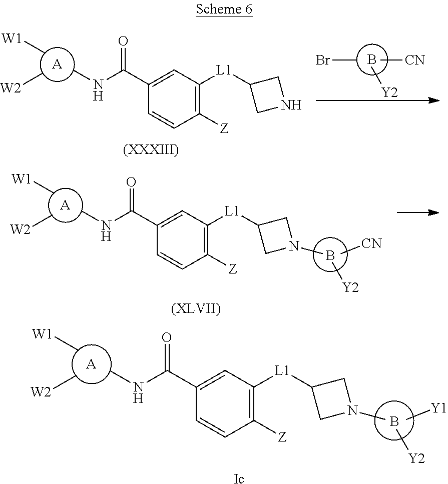

In a different approach, compounds of formula (Ic) can be prepared as depicted in Scheme 6 from compounds of formula (XXXIII) by performing a Buchwald coupling reaction with the appropriate cyano-heteroaryl bromide, in the presence of a suitable palladium source/phosphine-based ligand at high temperature, in the presence of a suitable solvent, such as DMA, and an inorganic base, such as Cs₂CO₃, to give compounds of formula (XLVII). Compounds of formula (Ic) may be obtained after conversion of the cyano group (CN) to primary amide in the presence of a peroxide, such as hydrogen peroxide, an inorganic base, such as K₂CO₃, and the suitable solvent, such as DMSO.

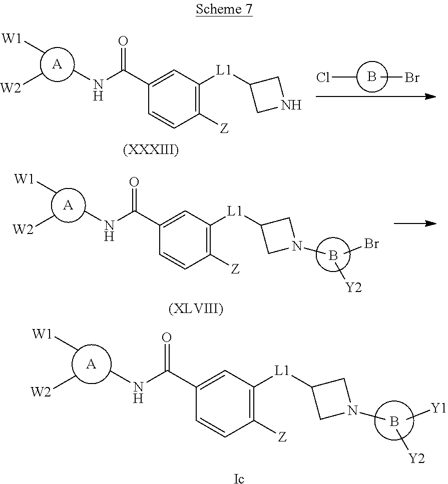

Alternatively, compounds of formula (XXXIII) can be converted into compounds of formula (XLVIII) through a nucleophilic aromatic substitution (S_{N}Ar) using the suitable commercially available chloro-heteroaryl in the presence of a suitable base, such as DIPEA, and the appropriate solvent, such as acetonitrile.

Compounds of formula (Ic) can be prepared as depicted in Scheme 7 by reacting a compound of formula (XLVIII) with an aryl or heteroaryl boronic ester/acid by following a common Suzuki protocol, in a suitable organic solvent, such as THF and water, in the presence of an inorganic base, such as K₃PO₄, with an appropriate palladium catalytic system, such as Pd-170, under heating (for instance around 50° C.) for few hours.

Accordingly, the present invention provides intermediate compounds of formula (XXI), (XXX), (XXXI), (XXXII), (XXXIII), (XXXV), (XXXVI), (XXXVII), (XXXVIII), (XXXIX), (XL), (XLI), (XLII), (XLIII), (XLIV), (XLV) and (XLVI), as defined above, and their use in the preparation of compounds of formula (Ic).

In particular, the present invention provides an intermediate compound of formula (XXXIII)

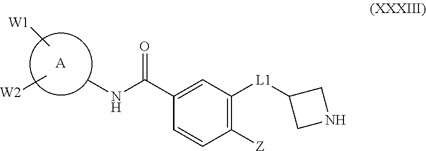

wherein
A is a ring selected from the group consisting of:

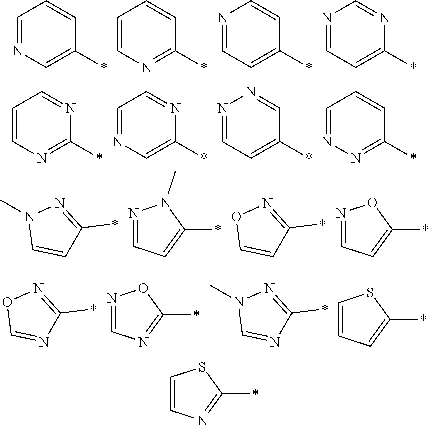

and phenyl, wherein [symbol] indicates a direct bond to NH;
W1 and W2 are substituents of ring A selected from the group consisting of hydrogen, halogen, (C₁-C₄)alkyl, (C₁-C₄)haloalkyl, (C₁-C₄)alkoxy, (C₁-C₄)haloalkoxy, R1R2N—(C₁-C₄)alkyl and (C₃-C₆)cycloalkyl;
R1 and R2 are independently selected from the group consisting of hydrogen, (C₁-C₄)alkyl, (C₁-C₄)hydroxyalkyl, (C₁-C₄)alkoxy(C₁-C₄)alkyl, (C₁-C₄)alkylamino-(C₁-C₄)alkyl, di-(C₁-C₄)alkylamino-(C₁-C₄)alkyl, optionally substituted (C₃-C₆)cycloalkyl, optionally substituted heterocycloalkyl and optionally substituted heterocycloalkyl-(C₁-C₄)alkoxy, wherein optional substituents are one or more and are selected from (C₁-C₄)alkyl, (C₁-C₄)alkoxy, (C₁-C₄)haloalkyl and (C₁-C₄)haloalkoxy;
Z is selected from the group consisting of (C₁-C₄)alkyl, (C₁-C₄)haloalkyl, (C₁-C₄)alkoxy, (C₁-C₄)haloalkoxy, (C₁-C₄)hydroxyalkyl and (C₃-C₆)cycloalkyl;
L1 is selected from the group consisting of O, NR3, CHR3, S and SO₂, or is absent;
R3 is selected from hydrogen and (C₁-C₄)alkyl;
or a stereoisomer, tautomer, solvate and pharmaceutically acceptable salt thereof.

Embodiments of the compound of formula (XXXIII) include:
a compound of formula (XXXVII), for L1 being NR3;
a compound of formula (XLIV), for L1 being CH3 or absent.

In further embodiments of the compound of formula (XXXIII) L1 is selected from the group consisting of O, S and SO₂, being preferably O.

In a preferred embodiment, the variables of formula (XXXIII) have the preferred meanings of the preferred compounds of formula (I) according to the invention.

The invention further provides the use of the compound of formula (XXXIII) as defined above in the preparation of a compound of formula (Ic).

The present invention also provides an intermediate compound of formula (XXXIX)

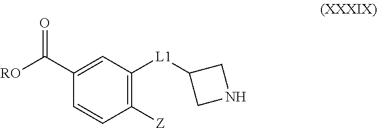

wherein
L1 is selected from the group consisting of O, NR3, CHR3, S and SO₂, or is absent;
R is (C₁-C₄)alkyl, preferably ethyl, and
Z is selected from the group consisting of (C₁-C₄)alkyl, (C₁-C₄)haloalkyl, (C₁-C₄)alkoxy, (C₁-C₄)haloalkoxy, (C₁-C₄)hydroxyalkyl and (C₃-C₆)cycloalkyl;
or a stereoisomer, tautomer, solvate and pharmaceutically acceptable salt thereof.

The invention further provides the use of the compound of formula (XXXIX) as defined above in the preparation of a compound of formula (Ic).

The present invention also provides an intermediate compound of formula (XL)

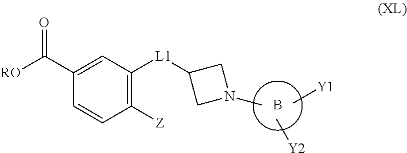

wherein
L1 is selected from the group consisting of O, NR3, CHR3, S and SO₂, or is absent;
R is (C₁-C₄)alkyl, preferably ethyl;
Z is selected from the group consisting of (C₁-C₄)alkyl, (C₁-C₄)haloalkyl, (C₁-C₄)alkoxy, (C₁-C₄)haloalkoxy, (C₁-C₄)hydroxyalkyl and (C₃-C₆)cycloalkyl;
B is mono- or bi-cyclic heteroaryl ring;
Y1 and Y2 are substituents of ring B independently selected from the group consisting of hydrogen, (C₁-C₄)alkyl, (C₁-C₄)haloalkyl, (C₁-C₄)alkoxy, (C₁-C₄)alkoxy-(C₁-C₄)alkoxy, hydroxy-(C₁-C₄)alkoxy, hydroxy-(C₁-C₄)alkyl, (C₁-C₄)alkyl-heterocycloalkyl-(C₀-C₄)alkoxy, (C₁-C₄)haloalkoxy, halogen, cyano, cyano-(C₁-C₄)alkyl, cyano-(C₁-C₄)alkyl-heterocycloalkyl, CONR1R2, NHCOR1, NR1R2, R1R2N—(C₁-C₄)alkyl, heterocycloalkyl, (C₁-C₄)alkyl-heterocycloalkyl, heterocycloalkyl-(C₁-C₄)alkyl, (C₁-C₄)alkyl-heterocycloalkyl-carbonyl, (C₀-C₄)alkyl-heterocycloalkyl-carbonyl (C₁-C₄)alkyl-phenyl and monocyclic (C₁-C₄)alkyl-heteroaryl;
R1 and R2 are independently selected from the group consisting of hydrogen, (C₁-C₄)alkyl, (C₁-C₄)hydroxyalkyl, (C₁-C₄)alkoxy(C₁-C₄)alkyl, (C₁-C₄)alkylamino-(C₁-C₄)alkyl, di-(C₁-C₄)alkylamino-(C₁-C₄)alkyl, optionally substituted (C₃-C₆)cycloalkyl, optionally substituted heterocycloalkyl and optionally substituted heterocycloalkyl-(C₁-C₄)alkoxy, wherein optional substituents are one or more and are selected from (C₁-C₄)alkyl, (C₁-C₄)alkoxy, (C₁-C₄)haloalkyl and (C₁-C₄)haloalkoxy;
R3 is selected from hydrogen and (C₁-C₄)alkyl, preferably hydrogen and methyl;
or a stereoisomer, tautomer, solvate and pharmaceutically acceptable salt thereof.

The invention further provides the use of the compound of formula (XL) as defined above in the preparation of a compound of formula (Ic).

Thus, the invention provides the use of the compound of formula (XXXIII) and/or the compound of formula (XXXIX) and/or the compound of formula (XL), as defined above, in the preparation of a compound of formula (Ic).

In preferred embodiments, the variables of formulas (XXXIII), (XXXIX) and (XL) have the preferred meanings of the preferred compounds of formula (I) according to the invention, respectively.

The compounds of formula (I) of the present invention have surprisingly been found to effectively inhibit both receptor DDR1 and DDR2. Advantageously, the inhibition of receptors DDR1 and DDR2 may result in efficacious treatment of the diseases, disorders or conditions wherein the DDR receptors are involved.

In this respect, it has been found that the compounds of formula (I) of the present invention have a very high antagonist drug potency on DDR1 and DDR2. Table 2 in the present experimental part reports such potency expressed as inhibition constant Ki for representative compounds of formula (I) of the invention. In particular, potencies wherein the Ki is below 100 nM are reported. Preferred compounds of the present invention have a Ki on DDR1 and DDR2 between 25 and 5 nM. Even more preferred compounds of the present invention have a Ki on DDR1 and DDR2 lower than 5 nM.

In one aspect, the present invention refers to a compound of formula (I) according to any of the embodiments disclosed above for use as a medicament.

In a preferred embodiment, the invention refers to a compound of formula (I), and pharmaceutically acceptable salts thereof, for use in treating diseases, disorders, or conditions associated with dysregulation of DDR.

In another aspect, the invention refers to the use of a compound of formula (I) as above described, and pharmaceutically acceptable salts thereof, in the preparation of a medicament for the treatment of disorders associated with dysregulation of DDR.

In another preferred embodiment, the invention refers to a compound of formula (I), and pharmaceutically acceptable salts thereof, for use in the prevention and/or treatment of a disease, disorder or condition associated with DDR receptor mechanism. In a more preferred embodiment, the present invention refers to a compound of formula (I) for use in the prevention and/or treatment of fibrosis and/or diseases, disorders or conditions that involve fibrosis.

The terms “fibrosis” or “fibrosing disorder,” as used herein, refer to conditions that are associated with the abnormal accumulation of cells and/or fibronectin and/or collagen and/or increased fibroblast recruitment and include, but are not limited to, fibrosis of individual organs or tissues such as the heart, kidney, liver, joints, lung, pleural tissue, peritoneal tissue, skin, cornea, retina, musculoskeletal and digestive tract.

Preferably, the compounds of formula (I) as above described are useful for the treatment and/or prevention of fibrosis, such as pulmonary fibrosis, idiopathic pulmonary fibrosis (IPF), hepatic fibrosis, renal fibrosis, ocular fibrosis, cardiac fibrosis, arterial fibrosis and systemic sclerosis.

More preferably, the compounds of formula (I) as above described are useful for the treatment of idiopathic pulmonary fibrosis (IPF).

In one aspect, the invention also refers to a method for the prevention and/or treatment of disorders associated with DDR receptors mechanisms, said method comprising the step of administering to a patient in need thereof a therapeutically effective amount of a compound of formula (I) as above described.

In a further aspect, the invention refers to the use of a compound of formula (I) as above described for the treatment of disorders associated with DDR receptors mechanism.

In another aspect, the invention refers to the use of a compound of formula (I) as above described in the preparation of a medicament for the treatment of disorders associated with DDR receptors mechanism.

In a further aspect, the invention refers to a method for the prevention and/or treatment of a disorder or condition associated with the dysregulation of DDR receptors 1 and 2, said method comprising administering to a patient in need of such treatment a therapeutically effective amount of a compound of formula (I) as above described.

In a further aspect, the present invention refers to the use of a compound of formula (I) as above described for the treatment of a disease, disorder or condition associated with dysregulation of DDR receptors 1 and 2.

As used herein, “safe and effective amount” in reference to a compound of formula (I) or a pharmaceutically acceptable salt thereof or other pharmaceutically active agent means an amount of the compound sufficient to treat the patient's condition but low enough to avoid serious side effects and that can nevertheless be routinely determined by the skilled artisan.

The compounds of formula (I) may be administered once or according to a dosing regimen wherein a number of doses are administered at varying intervals of time for a given period of time. Typical daily dosages may vary depending upon the route of administration chosen.

The present invention also refers to a pharmaceutical composition comprising a compound of formula (I) according to any of its embodiment in admixture with at least one or more pharmaceutically acceptable carrier and/or excipient.

In one embodiment, the invention refers to a pharmaceutical composition of compounds of formula (I) in admixture with at least one or more pharmaceutically acceptable carrier and/or excipient, for example those described in Remington's Pharmaceutical Sciences Handbook, XVII Ed., Mack Pub., N.Y., U.S.A.

Administration of the compounds of the invention and their pharmaceutical compositions may be accomplished according to patient needs, for example, orally, nasally, parenterally (subcutaneously, intravenously, intramuscularly, intrasternally and by infusion) and by inhalation.

Preferably, the compounds of the present invention are administered orally or by inhalation.

In one preferred embodiment, the pharmaceutical composition comprising the compound of formula (I) is a solid oral dosage form such as tablets, gelcaps, capsules, caplets, granules, lozenges and bulk powders.

In one embodiment, the pharmaceutical composition comprising the compound of formula (I) is a tablet.

The compounds of the invention can be administered alone or combined with various pharmaceutically acceptable carriers, diluents (such as sucrose, mannitol, lactose, starches) and known excipients, including suspending agents, solubilizers, buffering agents, binders, disintegrants, preservatives, colorants, flavorants, lubricants and the like.

In a further embodiment, the pharmaceutical composition comprising a compound of formula (I) is a liquid oral dosage form such as aqueous and non-aqueous solutions, emulsions, suspensions, syrups, and elixirs. Such liquid dosage forms can also contain suitable known inert diluents such as water and suitable known excipients such as preservatives, wetting agents, sweeteners, flavorants, as well as agents for emulsifying and/or suspending the compounds of the invention.

In a further embodiment, the pharmaceutical composition comprising the compound of formula (I) is an inhalable preparation such as inhalable powders, propellant-containing metering aerosols or propellant-free inhalable formulations.

For administration as a dry powder, single- or multi-dose inhalers known from the prior art may be utilized. In that case the powder may be filled in gelatine, plastic or other capsules, cartridges or blister packs or in a reservoir.

A diluent or carrier chemically inert to the compounds of the invention, e.g. lactose or any other additive suitable for improving the respirable fraction may be added to the powdered compounds of the invention.

Inhalation aerosols containing propellant gas such as hydrofluoroalkanes may contain the compounds of the invention either in solution or in dispersed form. The propellant-driven formulations may also contain other ingredients such as co-solvents, stabilizers and optionally other excipients.

The propellant-free inhalable formulations comprising the compounds of the invention may be in form of solutions or suspensions in an aqueous, alcoholic or hydroalcoholic medium and they may be delivered by jet or ultrasonic nebulizers known from the prior art or by soft-mist nebulizers.

The compounds of the invention can be administered as the sole active agent or in combination with other pharmaceutical active ingredients.

The dosages of the compounds of the invention depend upon a variety of factors including among others the particular disease to be treated, the severity of the symptoms, the route of administration and the like.

The invention is also directed to a device comprising a pharmaceutical composition comprising a compound of Formula (I) according to the invention, in form of a single- or multi-dose dry powder inhaler or a metered dose inhaler.

All preferred groups or embodiments described above for compounds of formula (I) may be combined with each other and apply as well mutatis mutandis.

The various aspects of the invention described in this application are illustrated by the following examples which are not meant to limit the invention in any way.

### PREPARATIONS OF INTERMEDIATES AND EXAMPLES

Chemical Names of the compounds were generated with Structure-To-Name tool of PerkinElmer ChemDraw Professional application (v. 20.0.0.41.). All reagents, for which the synthesis is not described in the experimental part, are either commercially available, or are known compounds or may be formed from known compounds by known methods by a person skilled in the art.

In the procedures that follow, some of the starting materials are identified through an “Intermediate” or “Example” number with indications on step number. This is provided merely for assistance to the skilled chemist.

When reference is made to the use of a “similar” or “analogous” procedure, as will be appreciated by those skilled in the art, such a procedure may involve minor variations, for example reaction temperature, reagent/solvent amount, reaction time, work-up conditions or chromatographic purification conditions. All compounds were obtained as a free base, unless stated otherwise.

### Abbreviations

AcOH=Acetic acid; Acetone-d₆=deuterated acetone; ACN=acetonitrile; ACN-d₃=deuterated acetonitrile; CDCl₃=deuterated chloroform; CV=Column Volumes; DCM=dichloromethane; DIPEA=N,N-Diisopropylethylamine; DMF=dimethylformamide; DMSO=dimethyl sulfoxide; DMSO-d₆=deuterated dimethyl sulfoxide; ee=enantiomeric excess; Et₂O=diethyl ether; EtOAc=Ethyl acetate; eq.=equivalents; FCC=flash column chromatography; h=hour/s; HATU=1-[Bis(dimethylamino)methylene]-1H-1,2,3-triazolo[4,5-b]pyridinium 3-oxid hexafluorophosphate; HCOOH=formic acid; HPLC=high pressure liquid chromatography; LC-MS=Liquid Chromatography/Mass Spectrometry; LDA=Lithium diisopropylamide; Na/LiHMDS=Sodium/Lithium bis(trimethylsilyl)amide; MeOH=methyl alcohol; min=minute/s; TBAF=Tetrabutylammonium fluoride; 2-MeTHF=2-Methyltetrahydrofuran; NaBH₃CN=sodium cyanoborohydride; NMR=nuclear magnetic resonance; MeOH-d₄=deuterated methanol; Pd(dba)₂=Bis(dibenzylideneacetone)palladium(0); Pd(dppf)Cl₂=[1,1′-Bis(diphenylphosphino)ferrocene]dichloropalladium(II); RT/rt=room temperature; RuPhos=2-Dicyclohexylphosphino-2′,6′-diisopropoxybiphenyl; SCX=strong cation exchange; SFC=supercritical fluid chromatography; STAB=sodium triacetoxyborohydride; SM=starting material; tBu=tert-Butyl; tBuBrettPhos Pd G3=[(2-Di-tert-butylphosphino-3,6-dimethoxy-2′,4′,6′-triisopropyl-1,1′-biphenyl)-2-(2′-amino-1,1′-biphenyl)]palladium(II) methanesulfonate generation 3; tBuOK=Potassium tert-butoxide; TBDMS=tert-butyldimethylsilyl; TBTU=2-(1H-Benzotriazole-1-yl)-1,1,3,3-tetramethylaminium tetrafluoroborate; TEA=triethylamine; TFA=trifluoroacetic acid; THF=tetrahydrofuran; TLC=Thin Layer Chromatography; t_{R}=retention time; UPLC=Ultra Performance Liquid Chromatography; VCD=Vibrational circular dichroism; XantPhos=4,5-Bis(diphenylphosphino)-9,9-dimethylxanthene; IPA=Isopropanol; WE=working electrode; CE=Counter electrode.

### General Experimental Details

### NMR Characterization:

¹H NMR spectra were recorded on Varian MR-400 spectrometer operating at 400 MHZ (proton frequency), equipped with: a self-shielded Z-gradient coil 5 mm 1H/nX broadband probe head for reverse detection, deuterium digital lock channel unit, quadrature digital detection unit with transmitter offset frequency shift. Chemical shifts are reported as δ values in ppm relative to tetramethyl silane (TMS) as an internal standard. Coupling constants (J values) are given in hertz (Hz) and multiplicities are reported using the following abbreviation (s=singlet, d=doublet, t=triplet, q=quartet, dd=doublet of doublets, dt=doublet of triplets, m=multiplet, br=broad, nd=not determined).

In some cases, signals NH from amide bond or amine bond (Exchangeable protons) are not visible. In a few cases, some signals could be hidden under the signal of water or under the signal of DMSO or other residual solvents.

### LC/UV/MS Analytical Methods

LC/MS retention times are estimated to be affected by an experimental error of ±0.5 min.

Method 1: Acquity CSH C18 column 50 mm×2.1 mm 1.7 μm, maintained at 40° C.; Mobile Phase: Eluent B (ACN/water 95:5+0.05% HCOOH) in Eluent A (water/ACN 95:5+0.05% HCOOH) from 1% to 99.9% within 1.5 min. Flow rate: 1 mL/min. Wavelength: 210-400 nm DAD. UPLC+Waters PDA+Waters QDA.

Method 2: Waters Sunfire C18 column, 4.6×50 mm, 3.5 μm, maintained at 40° C. Mobile phase ACN in water+10 mM ammonium bicarbonate, from 5 to 95% within 2.5 min. Flow rate: 2.0 mL/min. Wavelength: 210-400 nm DAD. Waters 2795 separations module+Waters DAD+Micromass ZQ, single quadrapole LC-MS.

Method 3: Acquity CSH C18 column 50 mm×2.1 mm 1.7 μm, maintained at 40° C.; Mobile Phase: Eluent B (ACN/water 95:5+0.05% HCOOH) in Eluent A (water/ACN 95:5+0.05% HCOOH) from 1% to 99.9% within 3.5 min. Flow rate: 1 mL/min. Wavelength: 210-400 nm DAD. UPLC+Waters PDA+Waters QDA.

Method 4: Waters Acquity QSM, Kinetex C8 column 100 mm×2.1 mm 1.7 μm, maintained at 55° C.; Mobile Phase: Eluent A (HCOONH4 0.025M pH 3), Eluent B (ACN+0.1% FA). Gradient mode: from 0 to 3 min. eluent B is increased from 1% to 30%, from 3 to 6.50 min. is increased from 30% to 50%, from 6.50 to 7.50 min. is increased from 50% to 80%, from 7.50 to 8 min is kept at 80%, from 8 to 8.10 min. is decreased from 80% to 1% and from 8.10 it is kept at 1% till the end at 10 min. Flow rate: 0.5 mL/min. Wavelength: 210-400 nm PAD. UPLC+Waters PDA+Xevo TQS MS instrument.

Method 5: Acquity UPLC HSS C18 column, 100×2.1 mm, 1.8 μm (Plus guard cartridge), maintained at 40° C. Mobile phase: ACN (0.1% formic acid) in water (0.1% formic acid) from 5% to 95% within 5.6 min. Flow rate: 0.4 ml/min. Wavelength: 210-400 nm DAD. UPLC+Waters DAD+Waters SQD2, single quadrapole UPLC-MS.

Method 6: Agilent Zorbax column 4.6×50 mm, 3.5 μm, maintained at 40° C. Mobile phase: ACN (0.1% formic acid) in water (0.1% formic acid), from 5% to 95% within 2 min. Flow rate: 3.0 mL/min. Wavelength: 210-400 nm DAD. Waters 2795/2695 separations module+Waters DAD+Micromass ZQ, single quadrapole LC-MS.

Method 7: Acquity BEH C18 (2.1 mm×50 mm, 1.7 um) maintained at 60° C.; Flow Rate 1.0 mL/min; Detector Wavelength 220-300 nm; Injection Volume 1.0 uL; Mobile Phase Eluent B ACN Eluent A Water (0.1% v/v TFA) from 2% to 98% within 2 min. Flow rate: 1 mL/min. Wavelength: 220-300 nm.

Method 8: Acquity UPLC BEH Shield RP18 column, 100×2.1 mm, 1.72 μm (Plus guard cartridge), maintained at 40° C. Mobile phase: ACN in water+10 nM ammonium bicarbonate from 5% to 95% within 5.6 min. Flow rate: 0.4 ml/min. Wavelength: 210-400 nm DAD. UPLC+Waters DAD+Waters SQD2, single quadrapole UPLC-MS.

Method 9: Kinetex® XB-C18 column, 4.6×50 mm, 2.6 μm maintained at 25° C. Mobile phase: water (0.1% formic acid) in MeCN (0.1% formic acid), from 80% to 5% within 3.90 min; Flow rate: 1.0 ml/min; wavelength: 190-340 nm DAD. Dionex UHPLC Ultimate 3000 with DAD detector/Thermo Scientific ISQ EC mass spectrometer.

Method 10: Kinetex® XB-C18 column, 4.6×50 mm, 2.6 μm maintained at 25° C. Mobile phase: water (0.1% formic acid) in MeCN (0.1% formic acid), from 90% to 5% within 3.90 min; Flow rate: 1.0 ml/min; wavelength: 190-340 nm DAD. Dionex UHPLC Ultimate 3000 with DAD detector/Thermo Scientific ISQ EC mass spectrometer.

Method 11: Kinetex® XB-C18 column, 4.6×50 mm, 2.6 μm maintained at 25° C. Mobile phase: water (0.1% formic acid) in MeCN (0.1% formic acid), from 60% to 5% within 3.90 min; Flow rate: 1.0 ml/min; wavelength: 190-340 nm DAD. Dionex UHPLC Ultimate 3000 with DAD detector/Thermo Scientific ISQ EC mass spectrometer.

Method 12: Acquity CSH C18 column 50 mm×2.1 mm 1.7 μm, maintained at 40° C.; Mobile Phase: Eluent B (ACN) in Eluent A (water+0.1% HCOOH) from 1% to 99.9% within 1.5 min. Flow rate: 1 mL/min. Wavelength: 210-400 nm DAD. UPLC+Waters PDA+Waters QDA.

Method 13: Acquity CSH C18 column 50 mm×2.1 mm 1.7 μm, maintained at 40° C.; Mobile Phase: Eluent B (ACN) in Eluent A (water+0.1% HCOOH) from 1% to 99.9% within 3.5 min. Flow rate: 1 mL/min. Wavelength: 210-400 nm DAD. UPLC+Waters PDA+Waters QDA.

Method 14: Waters™ Acquity QSM, Acquity UPLC CSH C18 column 50 mm×2.1 mm 1.7 μm, maintained at 50° C.; Mobile Phase: Eluent A (HCOONH₄ 0.025M pH 3), Eluent B (ACN+0.1% FA). Gradient mode: from 0 to 5.50 min. eluent B is increased from 20% to 80%, from 5.50 to 7.50 min. is kept at 80%, from 7.50 to 8 min. is decreased from 80% to 20%, and from 8 min. it is kept at 20% till the end at 10 min. Flow rate: 0.35 mL/min. Wavelength: 210-400 nm DAD. UPLC+Waters™ PDA+Xevo TQS MS instrument.

Method 15: Kinetex® XB-C18 column, 4.6×50 mm, 2.6 μm maintained at 25° C. Mobile phase: water (0.1% formic acid) in MeCN (0.1% formic acid), from 50% to 5% within 3.90 min; Flow rate: 1.0 ml/min; wavelength: 190-340 nm DAD. Dionex UHPLC Ultimate 3000 with DAD detector/Thermo Scientific ISQ EC mass spectrometer.

Method 16: Acquity UPLC BEH—Waters, 1.7 μm C18 (2.1×100 mm), 130 Å, maintained at 25° C. Mobile phase: water (0.1% formic acid) in MeCN (0.1% formic acid), from 80% to 5% within 2.70 min; Flow rate: 0.5 ml/min; wavelength: 254 nm. Shimadzu LCMS-2020 Single Quadrupole Liquid Chromatograph Mass Spectrometer.

### Chiral Supercritical Fluid Chromatography (SFC) Separation Protocol

The diastereomeric separation of compounds was achieved by Supercritical Fluid Chromatography (SFC) using a Gilson Preparative LC system. Analysis of two enantiomers was performed from reconstituted final samples.

Otherwise, the diastereomeric separation of compounds was achieved by Supercritical Fluid Chromatography (SFC) using a Waters Thar Prep100 preparative SFC system (P200 CO2 pump, 2545 modifier pump, 2998 UV/VIS detector, 2767 liquid handler with Stacked Injection Module). The Waters 2767 liquid handler acted as both auto-sampler and fraction collector. Appropriate isocratic methods were selected based on methanol, ethanol or isopropanol solvent systems under un-modified or basic conditions. The standard SFC method used was modifier, CO2, 100 mL/min, 120 Bar backpressure, 40° C. column temperature. The modifier used under basic conditions was diethylamine (0.1% V/V). The modifier used under acidic conditions was either formic acid (0.1% V/V) or trifluoroacetic acid (0.1% V/V). The SFC purification was controlled by Waters Fractionlynx software through monitoring at 210-400 nm and triggered at a threshold collection value, typically 260 nm. Collected fractions were analysed by SFC (Waters/Thar SFC systems with Waters SQD). The fractions that contained the desired product were concentrated by vacuum centrifugation.

### Supercritical Fluid Chromatography—Mass Spectrometry Analytical Conditions

Method 17: SFC-MS was performed on a Waters™/Thar SFC systems with Waters™ SQD using a LUX Cellulose-1 (20×250 mm, 5 μm) column with an isocratic run (30% MeOH (NH₄OH 0.1%):CO₂), flow rate 100 mL/min, 120 bar, 40° C. column temperature, DAD wavelength 265 nm.

All solvents were purchased from commercial sources and were used without additional purification. Flash chromatography (FCC) was performed on Biotage Isolera, then SCX (NH) was utilized to obtain free base of the product, unless differently stated.

For reverse phase FCC the following gradient/eluent were used: gradient A:B from 100:0 to 0:10 in 12 CV eluent A: H₂O/ACN/HCOOH 95:5:0.1 Eluent B: H₂O/ACN/HCOOH 5:95:0.1.

### General Synthetic Procedures

### Intermediate 4: N-(5-bromopyridin-3-yl)acetamide

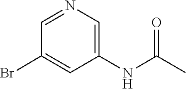

To a solution of 5-bromopyridin-3-amine (1 g, 5.78 mmol) in DCM (30 ml) acetic anhydride (1.745 ml, 18.50 mmol) was added followed by DIPEA (2.73 ml, 15.61 mmol). Solution was stirred at rt. On the following day, UPLC-MS analysis showed complete conversion of SM. Solution was diluted with NaHCO₃ sat solution (20 mL), extracted with DCM (20 mL×2) and then with EtOAc (20 mL). The organic layer was dried over MgSO₄ and under reduced pressure. The crude was purified by FCC on a silica gel column eluting with a gradient of 0-80% EtOAc in n-Heptane to provide the title compound (1.1 g, 5.17 mmol, yield 88%). LC-MS (ESI, m/z): method 1, t_{R}=0.56 min, m/z (M+1)=214.8/216.8

### Intermediate 6: 5-(tert-butyl)-1-methyl-1H-pyrazol-3-amine

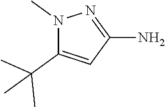

To a solution of 3-chloro-4,4-dimethylpent-2-enenitrile (5.00 g, 34.8 mmol, 1.00 eq) and K₂CO₃ (4812 mg, 34.8 mmol, 1.00 eq) in EtOH (25 mL) methylhydrazine (2.0 mL, 38.3 mmol, 1.10 eq) was added and the reaction mixture was heated at 80° C. for 4 h. The reaction mixture was cooled to rt, filtered through a Celite® pad and the filtrate was concentrated in vacuo. The residue was purified by column chromatography on silica gel, eluting with 0-100% EtOAc in cyclohexane followed by 0-100% MeOH in EtOAc to give a 5:1 mixture of the desired product:its regioisomer. The mixture was washed with cyclohexane and filtered. The obtained residue was separated by achiral SFC (YMC Cellulose-SC 20×250 mm, 5 μm 10/90 IPA (0.1% DEA)/CO₂, 100 ml/min, 120 bar, 40° C., DAD 230 nm), and dried in vacuo at 40° C. to give the title compound (2.75 g, 17.2 mmol, 49%).

LC-MS (ESI, m/z): (Method 6), t_{R}=2.09 min, m/z (M+1)=154.2

¹H NMR (400 MHz, CDCl₃) δ 5.40 (s, 1H), 3.76 (s, 3H), 3.32-3.31 (m, 2H), 1.32 (s, 9H).

### Intermediate 7: 5-(trifluoromethoxy)pyridin-3-amine Hydrochloride

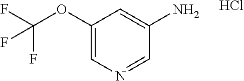 [SMILES: Cl ; Nc1cncc(OC(F)(F)F)c1]

### Step 1—tert-butyl N-[5-(trifluoromethoxy)-3-pyridyl]carbamate (Intermediate 8)

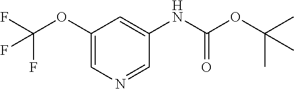 [SMILES: CC(C)(C)OC(=O)Nc1cncc(OC(F)(F)F)c1]

A mixture of tert-butyl carbamate (102 mg, 0.868 mmol, 1.20 eq), Xantphos (63 mg, 0.108 mmol, 0.150 eq), tris(dibenzylideneacetone)dipalladium(0)-chloroform adduct (37 mg, 0.0362 mmol, 0.0500 eq) and Cs₂CO₃ (283 mg, 0.868 mmol, 1.20 eq) in 1,4-dioxane (5 mL) was degassed with nitrogen and treated with 3-bromo-5-(trifluoromethoxy)pyridine (175 mg, 0.723 mmol, 1.00 eq). The reaction was stirred at 100° C. for 1 h. The reaction mixture was allowed to cool to rt, filtered through a pad of Celite®, which was then washed with dioxane, and the combined organic phases concentrated in vacuo. The residue was purified by column chromatography on silica gel, eluting with 0-100%, EtOAc in cyclohexane, then dried in vacuo overnight to afford the title compound (115 mg, 0.413 mmol, 57%).

LC-MS (ESI, m/z): (Method 2), t_{R}=1.24 min, m/z (M+1)=279.1

¹H NMR (400 MHz, CDCl3) δ 8.33 (d, J=2.3 Hz, 1H), 8.23-8.21 (m, 1H), 8.07 (s, 1H), 7.04 (s, 1H), 1.54 (s, 9H).

### Step 2—5-(trifluoromethoxy)pyridin-3-amine Hydrochloride (Intermediate 7)

HCl 4N in dioxane (3.0 mL, 0.413 mmol, 1.00 eq) was added to a solution of Intermediate 8 (115 mg, 0.413 mmol, 1.00 eq) in 1,4-dioxane (3 mL). The reaction mixture was stirred at rt overnight. The reaction mixture was diluted with diethyl ether (20 mL) and filtered to yield a white solid, washed with ether and dried in vacuo to afford the title compound (60 mg, 0.280 mmol, 68%). LC-MS (Method 2): t_{R}=0.88 min; m/z (M+1)=179.0.

¹H NMR (400 MHz, DMSO-d₆) δ 8.07 (d, J=2.0 Hz, 1H), 8.03 (d, J=1.5 Hz, 1H), 7.30 (d, J=1.0 Hz, 1H).

### Intermediate 40—3-bromo-6-(4-methylpiperazin-1-yl)imidazo[1,2-b]pyridazine

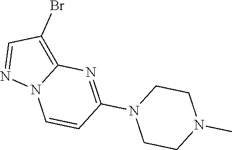 [SMILES: CN1CCN(c2ccn3ncc(Br)c3n2)CC1]

A suspension of 3-bromo-6-chloroimidazo[1,2-b]pyridazine (500 mg, 2.15 mmol), 1-methylpiperazine (0.72 mL, 6.45 mmol) and DIPEA (0.37 mL, 2.15 mmol) in DME (1.5 mL) was heated to 100° C. overnight. The reaction mixture was diluted in EtOAc and washed with NaHCO₃, the organic phase was dried over MgSO₄, filtered and concentrated in vacuo. The residue was purified by FCC (0-100% EtOAc in cyclohexane, followed by 10% 0.7 N NH₃ in MeOH in DCM) to give the title compound (610 mg, 2.06 mmol, 96%).

LC-MS (ESI): method 2, t_{R}=1.32 min; m/z (M+1)=296.0; 298.1

The following Intermediates were prepared using the procedure described for the synthesis of Intermediate 40.

| | | Product Amount |
|---|---|---|
| Intermediate | Structure | (Yield) |
| Nº | Analytical Data | |
| 41 | 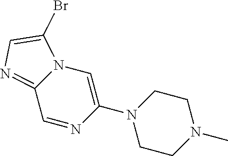 [SMILES: CN1CCN(c2cn3c(Br)cnc3cn2)CC1] | 261 mg (41%) |
| | LC-MS (ESI): method 2, t_{R} = 1.22 min; m/z (M + 1) = 296.1; 298.0 | |
| 42 | 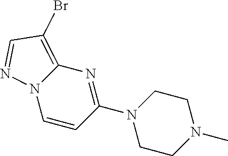 [SMILES: CN1CCN(c2ccn3ncc(Br)c3n2)CC1] | 499 mg (78%) |

LC-MS (ESI): method 2, t_{R} = 1.33 min, m/z (M + 1) = 296.1; 298.0

### Intermediate 43: N-(3-bromopyrazolo[1,5-a]pyrimidin-5-yl)acetamide

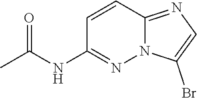

One chamber of a COware gas reactor was charged with a solution of 3-bromo-5-chloropyrazolo[1,5-a]pyrimidine (500 mg, 2.151 mmol) in THF (10 mL), then TEA (0.300 mL, 2.15 mmol) was added. The other one was charged with NH₃ water solution (5 mL, 73.4 mmol). the tubes were closed with sealed cap and the system was purged with nitrogen and placed under vacuum. The tubes were heated at 80° C. and stirred until complete conversion. The volatiles were removed under reduced pressure, then the residue was dissolved in pyridine (10 mL) and acetyl chloride (253 mg, 3.2 mmol) was added in one portion. The reaction mixture was diluted with DCM and the crude was washed with NaHCO₃, NH₄Cl and brine. The organic layer was dried over Na₂SO₄ and evaporated under vacuum. Title compound was obtained (336 mg, 1.32 mmol, 61% yield) and used as it is in the next step.

LC-MS (ESI): method 13 t_{R}=0.97 min; m/z (M+1)=254.9.0-226.8

### Example 142—4-methyl-3-((1-(pyrazolo[1,5-a]pyrimidin-3-yl)azetidin-3-yl)oxy)-N-(5-(trifluoromethyl)pyridin-3-yl)benzamide

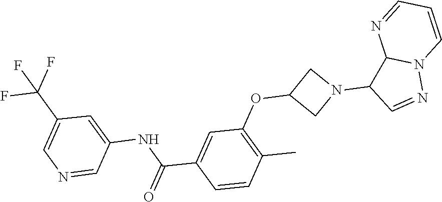 [SMILES: Cc1ccc(C(=O)Nc2cncc(C(F)(F)F)c2)cc1OC1CN(C2C=NN3C=CC=NC23)C1]

### Step 1: tert-butyl 3-(5-(ethoxycarbonyl)-2-methylphenoxy)azetidine-1-carboxylate (Intermediate 103)

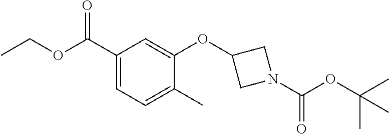 [SMILES: CCOC(=O)c1ccc(C)c(OC2CN(C(=O)OC(C)(C)C)C2)c1]

A ethyl 3-hydroxy-4-methylbenzoate (3 g, 16.7 mmol) and tert-butyl 3-hydroxyazetidine-1-carboxylate (3.75 g, 21.6 mmol) were dissolved in toluene (3 mL), then 2-(tributyl-lambda5-phosphanylidene)acetonitrile (6.54 mL, 24.97 mmol) in toluene (1.2 mL) was added. The reaction mixture was stirred at 70° C. overnight. The mixture was diluted with sat. NaHCO₃, then it was extracted with DCM. Organic layer was combined, dried over Na₂SO₄ and concentrated. The crude material was purified via FCC (100% Heptane to 100% AcOEt) to give title compound (5.5 g, 16.31 mmol, 89%).

LC-MS (ESI, m/z): method 13, t_{R}=2.53 min, m/z (M-tBu)=280.2

### Step 2: tert-butyl 3-(2-methyl-5-((5-(trifluoromethyl)pyridin-3-yl)carbamoyl) phenoxy)azetidine-1-carboxylate (Intermediate 104)

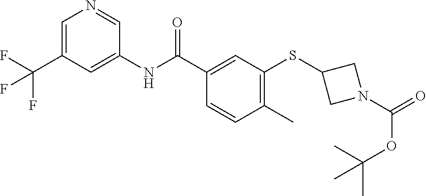 [SMILES: Cc1ccc(C(=O)Nc2cncc(C(F)(F)F)c2)cc1SC1CN(C(=O)OC(C)(C)C)C1]

5-(trifluoromethyl)pyridin-3-amine (0.873 g, 5.4 mmol) was dissolved in 1M LiHMDS in THF (16 mL, 16.1 mmol) and the solution was stirred at rt overnight. Next, intermediate 103 (1.8 g, 5.4 mmol) in THF (6 mL) was added and the reaction mixture was stirred for 2 h at rt; then it was diluted with water and extracted with AcOEt. All organic layers were combined, dried over Na₂SO₄, filtered and concentrated. Purification by FCC (Eluting with 100% CycHex to 50% AcOEt in CycHex) yielded the title compound (1.94 g, 4.30 mmol, 80%).

LC-MS (ESI, m/z): method 13, t_{R}=2.43 min, m/z (M+1)=452.1

Analogously, the following Intermediates were prepared by reacting suitable correspondent amine.

| Inter- | | Product Amount |
|---|---|---|
| mediate | Structure | (Yield) |
| Nº | Analytical Data | |
| 105 | 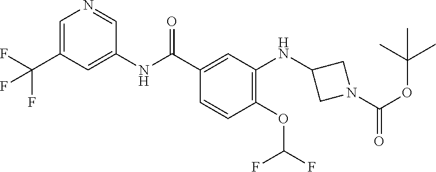 [SMILES: CC(C)(C)OC(=O)N1CC(Nc2cc(C(=O)Nc3cncc(C(F)(F)F)c3)ccc2OC(F)F)C1] | 2.6 g (84%) |
| | LC-MS (ESI, m/z): method 13, t_{R} = 2.29 min, m/z (M + 1 − tButyl) = 447.11 | |
| 106 | 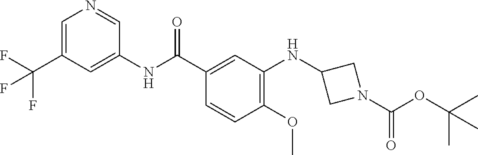 [SMILES: COc1ccc(C(=O)Nc2cncc(C(F)(F)F)c2)cc1NC1CN(C(=O)OC(C)(C)C)C1] | 3.4 g, (94%) |
| | LC-MS (ESI, m/z): method 13, t_{R} = 2.18 min, m/z (M + 1) = 467.13 | |
| 107 | 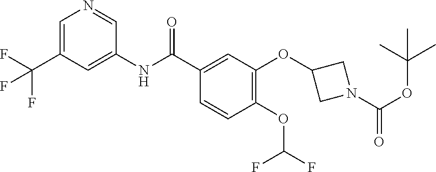 [SMILES: CC(C)(C)OC(=O)N1CC(Oc2cc(C(=O)Nc3cncc(C(F)(F)F)c3)ccc2OC(F)F)C1] | 920 mg, (83%) |
| | LC-MS (ESI, m/z): method 13, t_{R} = 2.34 min, m/z (M + 1 − tButyl) = 448.11 | |
| 108 | 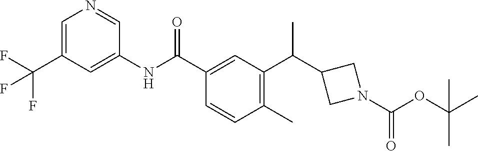 [SMILES: Cc1ccc(C(=O)Nc2cncc(C(F)(F)F)c2)cc1C(C)C1CN(C(=O)OC(C)(C)C)C1] | 165 mg (62%) |
| | LC-MS (ESI, m/z): method 13, t_{R} = 2.49 min, m/z (M + 1) = 464.23 | |
| 109 | 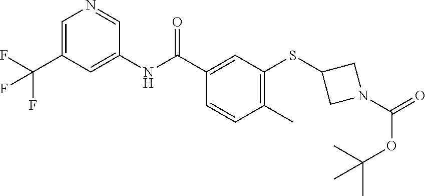 [SMILES: Cc1ccc(C(=O)Nc2cncc(C(F)(F)F)c2)cc1SC1CN(C(=O)OC(C)(C)C)C1] | 337 mg (82%) |
| | LC-MS (ESI, m/z): method 13, t_{R} = 2.51 min, m/z (M + 1) = 468.12 | |
| 110 | 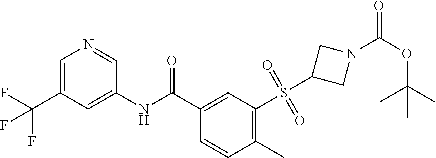 [SMILES: Cc1ccc(C(=O)Nc2cncc(C(F)(F)F)c2)cc1S(=O)(=O)C1CN(C(=O)OC(C)(C)C)C1] | 336 mg (86%) |
| | LC-MS (ESI, m/z): method 13, t_{R} = 2.14 min, m/z (M + 1) = 500.04 | |
| 137 | 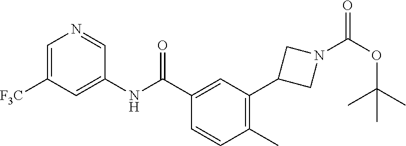 [SMILES: Cc1ccc(C(=O)Nc2cncc(C(F)(F)F)c2)cc1C1CN(C(=O)OC(C)(C)C)C1] | 270 mg (68%) |

LC-MS (ESI, m/z): method 13, t_{R} = 2.35 min, m/z (M + 1) = 436.14

### Step 3: 3-(azetidin-3-yloxy)-4-methyl-N-(5-(trifluoromethyl)pyridin-3-yl)benzamide (Intermediate 111)

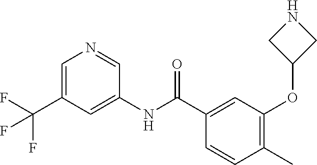 [SMILES: Cc1ccc(C(=O)Nc2cncc(C(F)(F)F)c2)cc1OC1CNC1]

Intermediate 104 (1.94 g, 4.30 mmol) was dissolved in DCM (21.5 mL) and TFA (3.3 mL, 43 mmol) was added dropwise. Reaction was stirred at rt for 3 h. Reaction mixture was quenched with sat. NaHCO₃ and extracted with CHCl₃:i-PrOH. All organic layers were combined, dried over Na₂SO₄, filtered and concentrated. Purification via FCC (DCM/2M NH₃ in MeOH from 100/0 to 80/20) gave desired product (1.35 g, 3.8 mmol, 89%).

LC-MS (ESI, m/z): method 13, t_{R}=0.91 min, m/z (M+1)=352.2

Analogously, the following Intermediates were prepared by reacting suitable analogue.

| Inter- | | Product Amount |
|---|---|---|
| mediate | Structure | (Yield) |
| Nº | Analytical Data | |
| 112 | 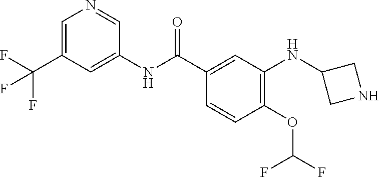 [SMILES: O=C(Nc1cncc(C(F)(F)F)c1)c1ccc(OC(F)F)c(NC2CNC2)c1] | 2.1 g (100%) |
| | LC-MS (ESI, m/z): method 12, t_{R} = 0.6 min, m/z (M + 1) = 403.08 | |
| 113 | 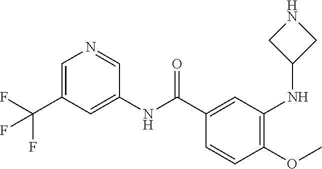 [SMILES: COc1ccc(C(=O)Nc2cncc(C(F)(F)F)c2)cc1NC1CNC1] | 2.6 g (100%) |
| | LC-MS (ESI, m/z): method 13, t_{R} = 0.81 min, m/z (M + 1) = 367.12 | |
| 114 | 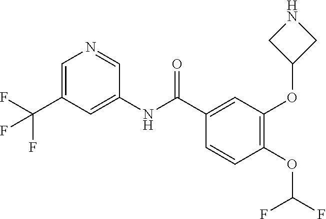 [SMILES: O=C(Nc1cncc(C(F)(F)F)c1)c1ccc(OC(F)F)c(OC2CNC2)c1] | 738 mg (100%) |
| | LC-MS (ESI, m/z): method 12, t_{R} = 0.59 min, m/z (M + 1) = 404.0 | |
| 115 | 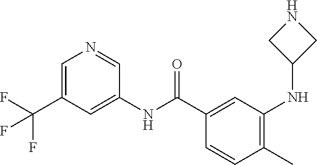 [SMILES: Cc1ccc(C(=O)Nc2cncc(C(F)(F)F)c2)cc1NC1CNC1] | 1.9 g (100%) |
| | LC-MS (ESI, m/z): method 12, t_{R} = 0.57 min, m/z (M + 1) = 351.1 | |
| 116 | 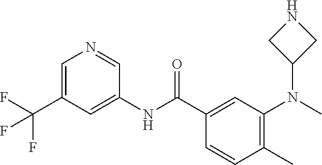 [SMILES: Cc1ccc(C(=O)Nc2cncc(C(F)(F)F)c2)cc1N(C)C1CNC1] | 790 mg (77%) |
| | LC-MS (ESI): method 12, t_{R} = 0.62 min, m/z (M + 1) = 365.2 | |
| 117 | 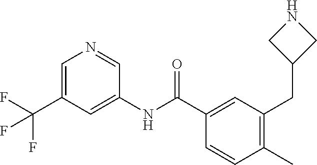 [SMILES: Cc1ccc(C(=O)Nc2cncc(C(F)(F)F)c2)cc1CC1CNC1] | 179 mg (quantitative) |
| | LC-MS (ESI): method 12, t_{R} = 0.57 min, m/z (M + 1) = 350.10 | |
| 118 | 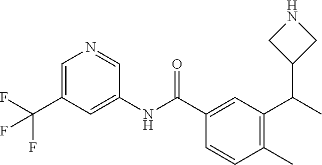 [SMILES: Cc1ccc(C(=O)Nc2cncc(C(F)(F)F)c2)cc1C(C)C1CNC1] | 122 mg (quantitative) |
| | LC-MS (ESI): method 12, t_{R} = 0.62 min, m/z (M + 1) = 364.17 | |
| 119 | 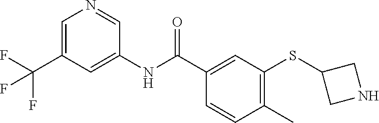 [SMILES: Cc1ccc(C(=O)Nc2cncc(C(F)(F)F)c2)cc1SC1CNC1] | 264 mg (quantitative) |
| | LC-MS (ESI): method 12, t_{R} = 0.60 min, m/z (M + 1) = 368.11 | |
| 120 | 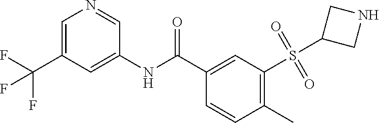 [SMILES: Cc1ccc(C(=O)Nc2cncc(C(F)(F)F)c2)cc1S(=O)(=O)C1CNC1] | 325 mg (quantitative) |
| | LC-MS (ESI): method 12, t_{R} = 0.57 min, m/z (M + 1) = 400.0 | |
| 138 | 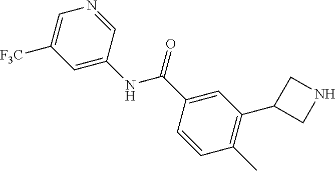 [SMILES: Cc1ccc(C(=O)Nc2cncc(C(F)(F)F)c2)cc1C1CNC1] | 208 mg (quantitative) |

LC-MS (ESI): method 12, t_{R} = 0.55 min, m/z (M + 1) = 336.07

The following Example was prepared by following the same procedure:

| | | Product Amount |
|---|---|---|
| Example | Structure | (Yield) |
| Nº | Analytical Data | |
| 180 | 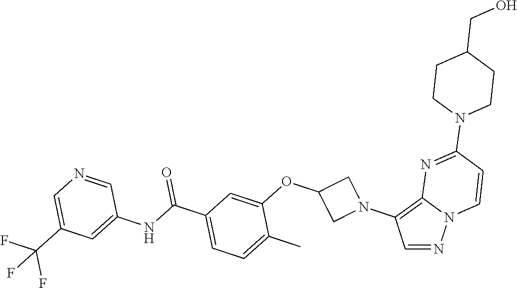 [SMILES: Cc1ccc(C(=O)Nc2cncc(C(F)(F)F)c2)cc1OC1CN(c2cnn3ccc(N4CCC(CO)CC4)nc23)C1] | 5 mg (20 %) |

LC-MS (ESI): Method 4 min, t_{R} = 1.46 min, m/z (M + 1) = = 583
¹H NMR (400 MHz, DMSO-d₆) δ ppm 10.67 (1 H, s), 9.20 (1 H, d, J = 2.19 Hz), 8.72 (1 H, s), 8.63 (1 H, s),
8.43 (1 H, d, J = 7.89 Hz), 7.57 (2 H, m), 7.39 (1 H, d, J = 7.89 Hz), 7.31 (1 H, s), 6.61 (1 H, d, J = 7.89 Hz),
5.20 (1 H, t, J = 5.26 Hz), 4.46 (1 H, t, J = 5.37 Hz), 4.37 (4 H, m), 3.86 (1 H, br d, J = 8.33 Hz), 3.84 (1 H,
br d, J = 8.33 Hz), 3.26 (2 H, t, J = 5.70 Hz), 2.88 (2 H, br t, J = 11.84 Hz), 2.27 (3 H, m), 1.71 (2 H, br d,
J = 12.93 Hz), 1.65 (1 H, m), 1.12 (2 H, m)

### Step 4: 4-methyl-3-((1-(pyrazolo[1,5-a]pyrimidin-3-yl)azetidin-3-yl)oxy)-N-(5-(trifluoromethyl)pyridin-3-yl)benzamide

### Example 142

A 0.5-2 mL microwave vial was charged with Intermediate 111 (100 mg, 0.28 mmol), 3-bromopyrazolo[1,5-a]pyrimidine (56 mg, 0.28 mmol), sodium tert-butoxide (82 mg, 0.85 mmol), BPC-305 (27.0 mg, 0.028 mmol) and Me-THF (2 mL). Mixture was stirred at 80° C. for 1 h. Solution was diluted with DCM and washed with water. Organic phase was evaporated under vacuum. Purification by RF FCC yielded the title compound (72 mg, 0.154 mmol, 54%).

LC-MS (ESI, m/z): method 14, t_{R}=5.95 min, m/z (M+1)=469.2

¹H NMR (acetone-d₆, 400 MHz) δ 10.05 (br s, 1H), 9.18 (d, 1H, J=2.2 Hz), 8.75 (s, 1H), 8.6-8.7 (m, 2H), 8.21 (dd, 1H, J=1.6, 3.8 Hz), 7.72 (s, 1H), 7.61 (dd, 1H, J=1.3, 7.7 Hz), 7.42 (s, 1H), 7.33 (d, 1H, J=7.9 Hz), 6.78 (dd, 1H, J=3.8, 7.1 Hz), 5.31 (t, 1H, 1=5.5 Hz), 4.6-4.6 (m, 2H), 4.01 (dd, 2H, J=4.8, 8.8 Hz), 2.29 (s, 3H)

Analogously, the following Examples or Intermediates were prepared by reacting the suitable analogues.

| Example/ | | Product | Catalyst- |
|---|---|---|---|
| Inter- | | Amount | Ligand |
| mediate | Structure | (Yield) | Base Solvent |
| Nº | Analytical Data | | |
| 134 | 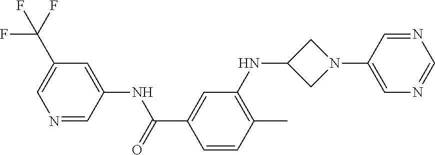 [SMILES: Cc1ccc(C(=O)Nc2cncc(C(F)(F)F)c2)cc1NC1CN(c2cncnc2)C1] | 25 mg (20%) | RuPhos, Pd(dppf)Cl₂ Cs₂CO₃ DMA |
| | LC-MS (ESI, m/z): method 9, t_{R} = 2.79 min, m/z (M + 1) = 429.15 | | |
| | ¹H NMR (400 MHz, DMSO-d₆) δ 10.58 (s, 1H), 9.21 (d, J = 2.3 Hz, 1H), 8.69 (t, J = 1.5 Hz, 1H), 8.67- | | |
| | 8.61 (m, 1H), 8.55 (s, 1H), 8.11 (s, 2H), 7.30 (dd, J = 7.5, 1.7 Hz, 1H), 7.20 (d, J = 7.7 Hz, 1H), 7.00 (d, | | |
| | J = 1.7 Hz, 1H), 5.74 (d, J = 7.0 Hz, 1H), 4.57 (h, J = 6.1 Hz, 1H), 4.43 (t, J = 7.3 Hz, 2H), 3.87 (dd, J = | | |
| | 7.7, 5.4 Hz, 2H), 2.21 (s, 3H). | | |
| 135 | 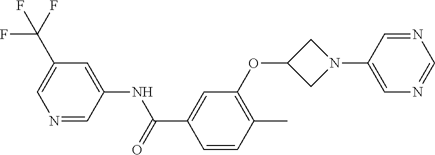 [SMILES: Cc1ccc(C(=O)Nc2cncc(C(F)(F)F)c2)cc1OC1CN(c2cncnc2)C1] | 30 mg (25%) | RuPhos, Pd(dppf)Cl₂ Cs₂CO₃ DMA |
| | LC-MS (ESI, m/z): method 9, t_{R} = 3.11 min, m/z (M + 1) = 430.17 | | |
| | ¹H NMR (400 MHz, DMSO-d₆) δ 10.70 (s, 1H), 9.18 (d, J = 2.3 Hz, 1H), 8.69 (d, J = 2.0 Hz, 1H), 8.64 | | |
| | (t, J = 2.4 Hz, 1H), 8.57 (s, 1H), 8.13 (s, 2H), 7.63 (dd, J = 7.7, 1.6 Hz, 1H), 7.39 (d, J = 7.8 Hz, 1H), | | |
| | 7.29 (d, J = 1.6 Hz, 1H), 5.33 (dd, J = 7.1, 3.4 Hz, 1H), 4.50 (dd, J = 8.8, 6.1 Hz, 2H), 3.99 (dd, J = | | |
| | 8.9, 3.9 Hz, 2H), 2.28 (s, 3H). | | |
| 136 | 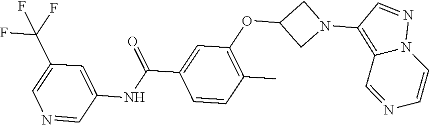 [SMILES: Cc1ccc(C(=O)Nc2cncc(C(F)(F)F)c2)cc1OC1CN(c2cnn3ccncc23)C1] | 46 mg (35%) | tBuBrettPhos Pd G3 tBuONa Dioxane |
| | LC-MS (ESI, m/z): method 9, t_{R} = 2.94 min, m/z (M + 1) = 469.25 | | |
| | ¹H NMR (400 MHz, DMSO-d₆) δ 10.69 (s, 1H), 9.21 (d, J = 2.3 Hz, 1H), 9.02 (d, J = 1.4 Hz, 1H), 8.71 (d, | | |
| | J = 2.0 Hz, 1H), 8.65 (d, J = 2.4 Hz, 1H), 8.47 (dd, J = 4.9, 1.4 Hz, 1H), 7.70 (s, 1H), 7.64-7.59 (m, 2H), | | |
| | 7.42-7.38 (m, 1H), 7.31 (d, J = 1.7 Hz, 1H), 5.32 (p, J = 5.4 Hz, 1H), 4.57 (dd, J = 8.2, 6.1 Hz, 2H), 4.01 | | |
| | (dd, J = 8.4, 4.4 Hz, 2H), 2.28 (s, 3H). | | |
| 137 | 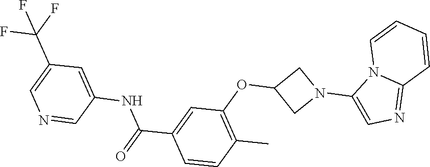 [SMILES: Cc1ccc(C(=O)Nc2cncc(C(F)(F)F)c2)cc1OC1CN(c2cnc3ccccn23)C1] | 16 mg (15%) | tBuBrettPhos Pd G3 tBuONa Dioxane |
| | LC-MS (ESI, m/z): method 4, t_{R} = 5.31 min, m/z (M + 1) = 468.31 | | |
| | ¹H NMR (400 MHz, acetone-d₆) δ ppm 10.03 (1 H, br s), 9.17 (1 H, s), 8.75 (1 H, s), 8.66 (1 H, s), 8.13 (1 H, | | |
| | d, J = 6.80 Hz), 7.63 (1 H, d, J = 7.67 Hz), 7.44 (2 H, m), 7.37 (1 H, d, J = 7.89 Hz), 7.27 (1 H, s), 7.15 (1 H, | | |
| | m), 6.86 (1 H, t, J = 6.69 Hz), 5.35 (1 H, t, J = 5.59 Hz), 4.59 (2 H, t, J = 7.23 Hz), 4.03 (2 H, dd, J = 8.00, | | |
| | 5.15 Hz), 2.33 (3 H, s) | | |
| 138 | 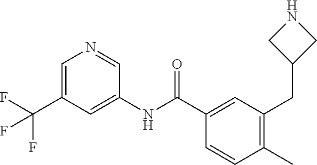 [SMILES: Cc1ccc(C(=O)Nc2cncc(C(F)(F)F)c2)cc1CC1CNC1] | 5 mg (4%) | tBuBrettPhos Pd G3 Cs₂CO₃ Me-THF |
| | LC-MS (ESI, m/z): method 4, t_{R} = 4.01 min, m/z (M + 1) = 526.26 | | |
| | ¹H NMR (400 MHz, DMSO-d₆) δ ppm 10.73 (1 H, s), 10.66 (1 H, s), 9.21 (1 H, d, J = 1.97 Hz), 8.77 (1 H, | | |
| | d, J = 7.67 Hz), 8.72 (1 H, s), 8.64 (1 H, s), 7.79 (1 H, s), 7.62 (1 H, dd, J = 7.67, 1.32 Hz), 7.57 (1 H, br d, | | |
| | J = 7.45 Hz), 7.39 (1 H, d, J = 7.89 Hz), 7.30 (1 H, s), 5.24 (1 H, quin, J = 5.32 Hz), 4.45 (2 H, dd, J = | | |
| | 7.67, 6.80 Hz), 3.87 (2 H, dd, J = 8.33, 4.82 Hz), 2.27 (3 H, s), 2.12 (3 H, s) | | |
| 140 | 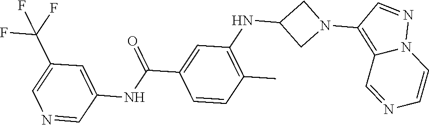 [SMILES: Cc1ccc(C(=O)Nc2cncc(C(F)(F)F)c2)cc1NC1CN(c2cnn3ccncc23)C1] | 57 mg (21%) | tBuBrettPhos Pd G3 tBuONa Dioxane |
| | LC-MS (ESI, m/z): method 9, t_{R} = 2.59 min, m/z (M + 1) = 468.26 | | |
| | ¹H NMR (400 MHz, DMSO-d₆) δ 10.58 (s, 1H), 9.20 (d, J = 2.3 Hz, 1H), 8.99 (d, J = 1.5 Hz, 1H), 8.71-8.61 | | |
| | (m, 2H), 8.45 (dd, J = 4.9, 1.4 Hz, 1H), 7.65 (s, 1H), 7.61 (d, J = 4.9 Hz, 1H), 7.29 (dd, J = 7.6, 1.7 Hz, 1H), | | |
| | 7.19 (d, J = 7.7 Hz, 1H), 7.04 (d, J = 1.7 Hz, 1H), 5.69 (d, J = 6.3 Hz, 1H), 4.54 (dq, J = 12.6, 6.6 Hz, 3H), | | |
| | 3.89 (t, J = 5.8 Hz, 2H), 2.20 (s, 3H). | | |
| 141 | 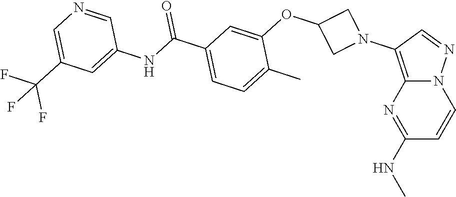 [SMILES: CNc1ccn2ncc(N3CC(Oc4cc(C(=O)Nc5cncc(C(F)(F)F)c5)ccc4C)C3)c2n1] | 5 mg (3%) | tBuBrettPhos Pd G3 Cs₂CO₃ Me-THF |
| | LC-MS (ESI, m/z): method 4, t_{R} = 5.70 min, m/z (M+1) = 498.27 | | |
| | ¹H NMR (400 MHz, DMSO-d₆) δ ppm 10.69 (1 H, br s), 8.94 (1 H, br s), 8.67 (1 H, s), 8.45 (1 H, br s), 8.27 | | |
| | (1 H, d, J = 7.45 Hz), 7.61 (1 H, d, J = 7.67 Hz), 7.50 (1 H, s), 7.39 (1 H, s), 7.26 (1 H, br d, J = 6.36 Hz), 7.20 | | |
| | (1 H, br d, J = 5.26 Hz), 6.10 (1 H, d, J = 7.45 Hz), 5.15 (1 H, br t, J = 5.26 Hz), 4.34 (2 H, m), 3.81 (2 H, dd, | | |
| | J = 8.11, 5.04 Hz), 2.81 (3 H, d, J = 4.60 Hz), 2.24 (3 H, s) | | |
| 183 | 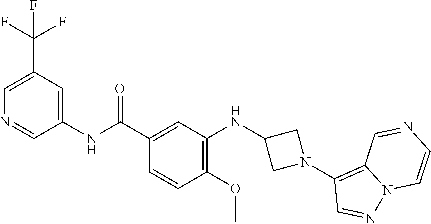 [SMILES: COc1ccc(C(=O)Nc2cncc(C(F)(F)F)c2)cc1NC1CN(c2cnn3ccncc23)C1] | 112 mg (28%) | BPC-304 Cs₂CO₃ Me-TH |
| | LC-MS (ESI, m/z): method 4, t_{R} = 5.9 min, m/z (M + 1) = 484.15 | | |
| | ¹H NMR (400 MHz, acetone-d₆) δ ppm 9.81 (1 H, br s), 9.16 (1 H, m), 8.93 (1 H, d, J = 1.32 Hz), 8.75 (1 H, | | |
| | s), 8.64 (1 H, s), 8.26 (1 H, dd, J = 4.82, 1.32 Hz), 7.60 (1 H, d, J = 4.82 Hz), 7.55 (1 H, s), 7.44 (1 H, dd, | | |
| | J = 8.33, 1.97 Hz), 7.23 (1 H, m), 6.99 (1 H, d, J = 8.33 Hz), 5.37 (1 H, m), 4.64 (1 H, m), 4.58 (2 H, m), | | |
| | 3.92 (5 H, m) | | |
| 182 | 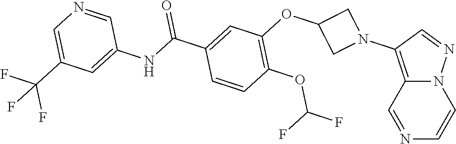 [SMILES: O=C(Nc1cncc(C(F)(F)F)c1)c1ccc(OC(F)F)c(OC2CN(c3cnn4ccncc34)C2)c1] | 30 mg (23%) | BPC-304 Cs₂CO₃ Me-THF |
| | LC-MS (ESI, m/z): method 4, t_{R} = 6.62 min, m/z (M + 1) = 521.10 | | |
| | ¹H NMR (400 MHz, acetone-d₆) δ ppm 10.06 (1 H, br s), 9.15 (1 H, s), 8.96 (1 H, d, J = 1.32 Hz), 8.71 (2 H, | | |
| | br d, J = 16.66 Hz), 8.28 (1 H, dd, J = 4.82, 1.32 Hz), 7.75 (1 H, dd, J = 8.33, 1.97 Hz), 7.62 (3 H, m), 7.40 (1 | | |
| | H, d, J = 8.33 Hz), 7.11 (1 H, t, J = 73.70 Hz), 5.43 (1 H, m), 4.67 (2 H, m), 4.15 (2 H, dd, J = 8.55, 4.60 Hz) | | |
| 146 | 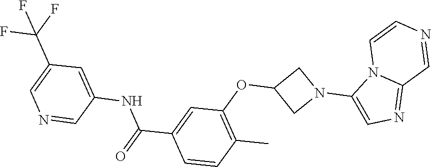 [SMILES: Cc1ccc(C(=O)Nc2cncc(C(F)(F)F)c2)cc1OC1CN(c2cnc3cnccn23)C1] | 43 mg (46%) | BPC-305 tBuONa Me-THF |
| | LC-MS (ESI, m/z): method 4, t_{R} = 5.47 min, m/z (M + 1) = 469.2 | | |
| | ¹H NMR (acetone-d₆, 400 MHz) δ 10.10 (br s, 1H), 9.19 (d, 1H, J = 2.2 Hz), 8.79 (d, 1H, J = 1.3 Hz), 8.76 (s, | | |
| | 1H), 8.64 (s, 1H), 8.10 (dd, 1H, J = 1.5, 4.8 Hz), 7.74 (d, 1H, J = 4.6 Hz), 7.63 (dd, 1H, J = 1.3, 7.7 Hz), 7.45 | | |
| | (s, 1H), 7.41 (s, 1H), 7.35 (d, 1H, J = 7.7 Hz), 5.4-5.4 (m, 1H), 4.7-4.7 (m, 2H), 4.14 (dd, 2H, J = 4.9, 8.7 Hz), | | |
| | 2.31 (s, 3H) | | |
| 151 | 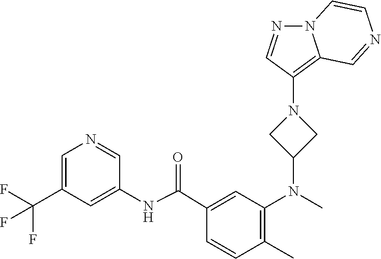 | 71 mg (38%) | Pd 175 Cs₂CO₃ Me-THF |
| | LC-MS (ESI, m/z): method 4, t_{R} = 6.72 min, m/z (M + 1) = 482.25 | | |
| | ¹H NMR (ACN-d₃, 400 MHz) δ 9.08 (d, 1H, J = 2.2 Hz), 9.02 (br s, 1H), 8.83 (d, 1H, J = 1.5 Hz), 8.66 (s, | | |
| | 1H), 8.61 (s, 1H), 8.14 (dd, 1H, J = 1.4, 4.9 Hz), 7.6-7.6 (m, 1H), 7.5-7.6 (m, 1H), 7.5-7.5 (m, 1H), 7.44 | | |
| | (s, 1H), 7.38 (d, 1H, J = 7.9 Hz), 4.4-4.4 (m, 1H), 4.29 (t, 2H, J = 6.9 Hz), 3.78 (t, 2H, J = 6.6 Hz), 2.66 | | |
| | (s, 3H), 2.39 (s, 3H) | | |
| 147 | 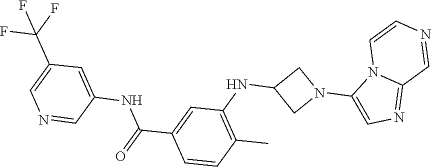 [SMILES: Cc1ccc(C(=O)Nc2cncc(C(F)(F)F)c2)cc1NC1CN(c2cnc3cnccn23)C1] | 21 mg (26%) | BPC-305 tBuONa Me-THF |
| | LC-MS (ESI, m/z): method 4, t_{R} = 4.93 min, m/z (M + 1) = 468.2 | | |
| | ¹H NMR (400 MHz, MeOH-d₄) δ ppm 9.12 (1 H, d, J = 2.20 Hz), 8.84 (1 H, d, J = 1.32 Hz), 8.67 (1 H, s), | | |
| | 8.62 (1 H, s), 8.16 (1 H, dd, J = 4.74, 1.43 Hz), 7.79 (1 H, d, J = 4.85 Hz), 7.42 (1 H, s), 7.31 (1 H, dd, J = | | |
| | 7.61, 1.65 Hz), 7.22 (1 H, d, J = 7.72 Hz), 7.12 (1 H, s), 4.67 (1 H, m), 4.60 (2 H, m), 3.98 (2 H, t, J = 6.50 | | |
| | Hz), 2.28 (3 H, m) | | |
| 181 | 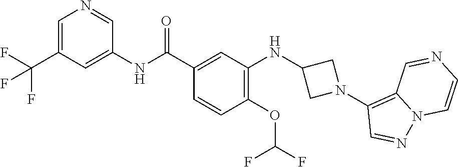 [SMILES: O=C(Nc1cncc(C(F)(F)F)c1)c1ccc(OC(F)F)c(NC2CN(c3cnn4ccncc34)C2)c1] | 55 mg (29%) | BPC-304 Cs₂CO₃ Me-THF |
| | LC-MS (ESI, m/z): method 4, t_{R} = 6.24 min, m/z (M + 1) = 520.20 | | |
| | ¹H NMR (400 MHz, acetone-d₆) δ ppm 9.95 (1 H, br s), 9.16 (1 H, s), 8.93 (1 H ,d, J = 1.53 Hz), 8.74 (1 H, | | |
| | s), 8.67 (1 H, s), 8.26 (1 H, dd, J = 4.93, 1.42 Hz), 7.60 (1 H, d, J = 4.82 Hz), 7.55 (1 H, s), 7.40 (1 H, m), | | |
| | 7.37 (1 H, m), 7.24 (1 H, d, J = 8.11 Hz), 7.03 (1 H, t, J = 73.70 Hz), 5.75 (1 H, br d, J = 7.02 Hz), 4.70 (1 | | |
| | H, m), 4.60 (2 H, t, J = 7.02 Hz), 3.97 (2 H, t, J = 6.47 Hz) | | |
| 148 | 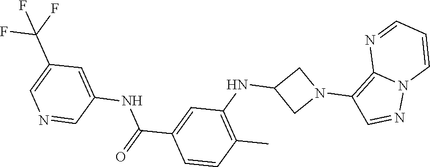 [SMILES: Cc1ccc(C(=O)Nc2cncc(C(F)(F)F)c2)cc1NC1CN(c2cnn3cccnc23)C1] | 13 mg (33%) | BPC-305 tBuONa Me-THF |
| | LC-MS (ESI, m/z): method 4, t_{R} = 5.21 min, m/z (M + 1) = 468.2 | | |
| | ¹H NMR (ACN-d₃, 400 MHz) δ 9.08 (d, 1H, J = 2.2 Hz), 9.00 (br s, 1H), 8.64 (s, 1H), 8.61 (s, 1H), 8.52 (dd, | | |
| | 1H, J = 1.5, 7.3 Hz), 8.19 (dd, 1H, J = 1.7, 3.9 Hz), 7.8-8.0 (m, 1H), 7.67 (s, 1H), 7.25 (dd, 1H, J = 1.5, 7.7 | | |
| | Hz), 7.20 (d, 1H, J = 7.7 Hz), 7.03 (s, 1H), 6.70 (dd, 1H, J = 3.9, 7.2 Hz), 4.5-4.7 (m, 3H), 3.8-3.9 (m, 2H), | | |
| | 2.2 (s, 3H) | | |
| 165 | 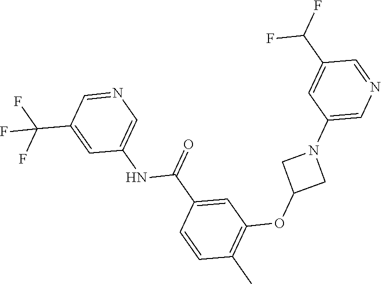 [SMILES: Cc1ccc(C(=O)Nc2cncc(C(F)(F)F)c2)cc1OC1CN(c2cncc(C(F)F)c2)C1] | 25 mg (23%) | RuPhos, Pd(dppf)Cl₂ Cs₂CO₃ DMA |
| | LC-MS (ESI, m/z): method 4, t_{R} = 4.85 min, m/z (M + 1) = 479.23 | | |
| | ¹H NMR (400 MHz, acetone-d₆) δ ppm 9.98 (1 H, br s), 9.16 (1 H, d, J = 2.19 Hz), 8.74 (1 H, s), 8.67 (1 H, | | |
| | s), 8.15 (1 H, m), 8.07 (1 H, m), 7.63 (1 H, dd, J = 7.67, 1.53 Hz), 7.39 (1 H, d, J = 1.32 Hz), 7.37 (1 H, d, | | |
| | J = 7.67 Hz), 6.96 (2 H, m), 5.41 (1 H, ddd, J = 6.14, 4.06, 2.08 Hz), 4.61 (2 H, dd, J = 8.44, 6.69 Hz), 4.07 | | |
| | (2 H, dd, J = 8.77, 3.95 Hz), 2.32 (3 H, s) | | |
| 166 | 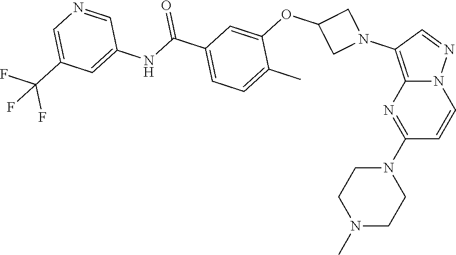 [SMILES: Cc1ccc(C(=O)Nc2cncc(C(F)(F)F)c2)cc1OC1CN(c2cnn3ccc(N4CCN(C)CC4)nc23)C1] | 5 mg (3%) | tBuBrettPhos Pd G3 Cs₂CO₃ Me-THF |
| | LC-MS (ESI, m/z): method 4, t_{R} = 4.71 min, m/z (M + 1) = 567.25 | | |
| | ¹H NMR (400 MHz, DMSO-d₆) δ ppm 10.67 (1 H, s), 9.20 (1 H, d, J = 1.97 Hz), 8.72 (1 H, s), 8.63 (1 H, s), | | |
| | 8.47 (1 H, d, J = 7.89 Hz), 7.60 (1 H, s), 7.58 (1 H, s), 7.39 (1 H, d, J = 7.67 Hz), 7.31 (1 H, s), 6.61 (1 H, d, | | |
| | J = 7.89 Hz), 5.21 (1 H, t, J = 5.48 Hz), 4.37 (2 H, t, J = 7.13 Hz), 3.84 (2 H, dd, J = 8.33, 5.04 Hz), 3.59 (4 | | |
| | H, m), 2.37 (4 H, br t, J = 4.82 Hz), 2.27 (3 H, s), 2.20 (3 H, s) | | |
| 167 | 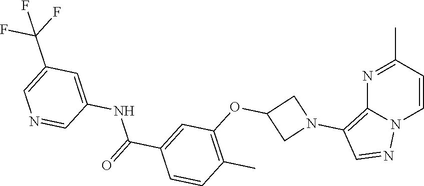 [SMILES: Cc1ccn2ncc(N3CC(Oc4cc(C(=O)Nc5cncc(C(F)(F)F)c5)ccc4C)C3)c2n1] | 13 g (12%) | Pd 175 Cs₂CO₃ Me-THF |
| | LC-MS (ESI, m/z): method 14, t_{R} = 4.57 min, m/z (M + 1) = 483.23 | | |
| | ¹H NMR (400 MHz, acetone-d₆) δ ppm 10.10 (1 H, br s), 9.19 (1 H, m), 8.76 (1 H, s), 8.66 (1 H, s), 8.51 (1 H, | | |
| | d, J = 7.45 Hz), 7.64 (2 H, m), 7.43 (1 H, s), 7.35 (1 H, d, J = 7.67 Hz), 6.68 (1 H, d, J = 7.45 Hz), 5.30 (1 H, | | |
| | t, J = 5.37 Hz), 4.57 (2 H, m), 3.99 (2 H, dd, J = 8.55, 4.82 Hz), 2.46 (3 H, s), 2.30 (3 H, s) | | |
| 168 | 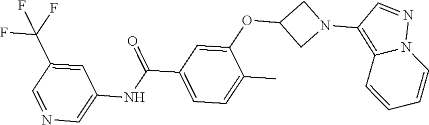 [SMILES: Cc1ccc(C(=O)Nc2cncc(C(F)(F)F)c2)cc1OC1CN(c2cnn3ccccc23)C1] | 71 g (27%) | tBuBrettPhos Pd G3 tBuONa Dioxane |
| | LC-MS (ESI, m/z): method 11, t_{R} = 2.64 min, m/z (M + 1) = 468.14 | | |
| | ¹H NMR (400 MHz, DMSO-d₆) δ 10.68 (s, 1H), 9.21 (d, J = 2.3 Hz, 1H), 8.72 (d, J = 1.9 Hz, 1H), 8.65 (d, J = | | |
| | 2.4 Hz, 1H), 8.44 (dt, J = 7.1, 1.1 Hz, 1H), 7.64 (s, 1H), 7.60 (dd, J = 7.8, 1.6 Hz, 1H), 7.55 (dt, J = 9.0, 1.3 | | |
| | Hz, 1H), 7.39 (d, J = 7.8 Hz, 1H), 7.32 (d, J = 1.7 Hz, 1H), 6.94 (ddd, J = 8.9, 6.6, 1.1 Hz, 1H), 6.71 (td, J = | | |
| | 6.8, 1.4 Hz, 1H), 5.27 (p, J = 5.5 Hz, 1H), 4.50-4.43 (m, 2H), 3.86 (dd, J = 8.2, 4.6 Hz, 2H), 2.28 (s, 3H). | | |
| 169 | 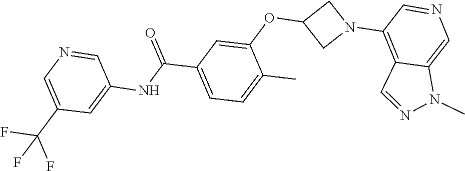 [SMILES: Cc1ccc(C(=O)Nc2cncc(C(F)(F)F)c2)cc1OC1CN(c2cncc3c2cnn3C)C1] | 4 mg (4%) | tBuBrettPhos Pd G3 Cs₂CO₃ Me-THF |
| | LC-MS (ESI, m/z): method 4, t_{R} = 5.52 min, m/z (M + 1) = 483.28 | | |
| | ¹H NMR (400 MHz, DMSO-d₆) δ ppm 10.70 (1 H, s), 9.22 (1 H, d, J = 1.97 Hz), 8.72 (1 H, s), 8.65 (1 H, s), | | |
| | 8.45 (1 H, s), 8.19 (1 H, s), 7.62 (1 H, d, J = 7.39 Hz), 7.41 (1 H, d, J = 7.89 Hz), 7.34 (2 H, m), 5.39 (1 H, br | | |
| | t, J = 3.95 Hz), 4.73 (2 H, dd, J = 8.22, 6.47 Hz), 4.20 (2 H, dd, J = 8.77, 3.73 Hz), 4.08 (3 H, s), 2.30 (3 H, m) | | |
| 170 | 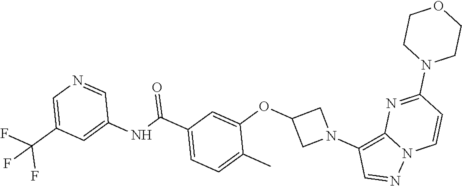 [SMILES: Cc1ccc(C(=O)Nc2cncc(C(F)(F)F)c2)cc1OC1CN(c2cnn3ccc(N4CCOCC4)nc23)C1] | 15 mg (19%) | tBuBrettPhos Pd G3 Cs₂CO₃ Me-THF |
| | LC-MS (ESI, m/z): method 4, t_{R} = 6.47 min, m/z (M+1) = 554.21 | | |
| | ¹H NMR (400 MHz, DMSO-d₆) δ ppm 10.67 (1 H, s), 9.20 (1 H, d, J = 1.97 Hz), 8.71 (1 H, s), 8.63 (1 H, s), | | |
| | 8.51 (1 H, d, J = 7.89 Hz), 7.58 (2 H, m), 7.38 (1 H, d, J = 7.89 Hz), 7.31 (1 H, s), 6.61 (1 H, d, J = 7.89 Hz), | | |
| | 5.21 (1 H, t, J = 5.37 Hz), 4.37 (2 H, t, J = 7.13 Hz), 3.84 (2 H, dd, J = 8.11, 5.04 Hz), 3.68 (4 H, m), 3.57 (4 | | |
| | H, m), 2.27 (3 H, s) | | |
| 171 | 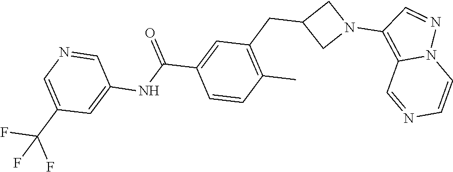 [SMILES: Cc1ccc(C(=O)Nc2cncc(C(F)(F)F)c2)cc1CC1CN(c2cnn3ccncc23)C1] | 36 mg (13%) | tBuBrettPhos Pd G3 Cs₂CO₃ Me-THF |
| | LC-MS (ESI, m/z): method 4, t_{R} = 6.52 min, m/z (M + 1) = 467.26 | | |
| | ¹H NMR (600 MHz, acetone-d₆) δ ppm 9.96 (1 H, br s), 9.16 (1 H, d, J = 2.35 Hz), 8.87 (1 H, d, J = 1.47 Hz), | | |
| | 8.74 (1 H, t, J = 1.98 Hz), 8.65 (1 H, s), 8.24 (1 H, dd, J = 4.99, 1.47 Hz), 7.91 (1 H, d, J = 1.91 Hz), 7.84 (1 | | |
| | H, dd, J = 7.78, 1.91 Hz), 7.58 (1 H, d, J = 4.84 Hz), 7.50 (1 H, s), 7.37 (1 H, d, J = 7.92 Hz), 4.19 (2 H, t, | | |
| | J = 7.26 Hz), 3.82 (2 H, m), 3.25 (1 H, m), 3.16 (2 H, d, J = 7.78 Hz), 2.44 (3 H, s) | | |
| 172 | 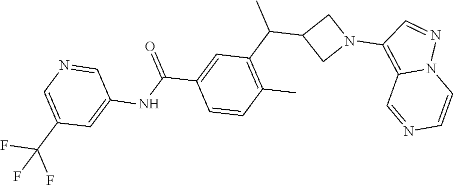 [SMILES: Cc1ccc(C(=O)Nc2cncc(C(F)(F)F)c2)cc1C(C)C1CN(c2cnn3ccncc23)C1] | 29 mg (22%) | BrettPhos Pd G3 Cs₂CO₃ Me-THF |
| | LC-MS (ESI, m/z): method 13, tR = 1.90 min, m/z (M + 1) = 481.10 | | |
| | ¹H NMR (400 MHz, acetone-d₆) δ ppm 9.94 (1 H, m), 9.15 (1 H, s), 8.85 (1 H, d, J = 1.32 Hz), 8.74 (1 H, s), | | |
| | 8.66 (1 H, s), 8.23 (1 H, dd, J = 4.82, 1.32 Hz), 7.96 (1 H, d, J = 1.53 Hz), 7.82 (1 H, dd, J = 7.89, 1.75 Hz), | | |
| | 7.57 (1 H, d, J = 4.82 Hz), 7.46 (1 H, s), 7.37 (1 H, d, J = 7.89 Hz), 4.34 (1 H, t, J = 7.13 Hz), 3.99 (1 H, t, | | |
| | J = 7.13 Hz), 3.91 (1 H, t, J = 6.36 Hz), 3.48 (2 H, m), 3.32 (1 H, m), 2.49 (3 H, s), 1.26 (3 H, d, J = 6.80 Hz) | | |
| 174 | 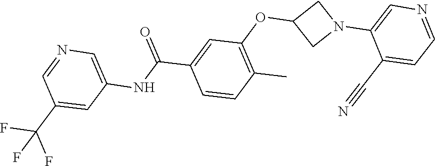 [SMILES: Cc1ccc(C(=O)Nc2cncc(C(F)(F)F)c2)cc1OC1CN(c2cnccc2C#N)C1] | 12 mg (12%) | RuPhos, Pd(dppf)Cl₂ Cs₂CO₃ DMA |
| | LC-MS (ESI, m/z): method 14, t_{R} = 4.77 min, m/z (M + 1) = 454.20 | | |
| | ¹H NMR (400 MHz, DMSO-d₆) δ ppm 10.71 (1 H, s), 9.20 (1 H, d, J = 2.19 Hz), 8.72 (1 H, s), 8.64 (1 H, s), | | |
| | 8.14 (1 H, s), 7.99 (1 H, d, J = 5.04 Hz), 7.63 (1 H, m), 7.50 (1 H, d, J = 5.04 Hz), 7.42 (1 H, d, J = 8.11 Hz), | | |
| | 7.28 (1 H, s), 5.33 (1 H, br t, J = 3.62 Hz), 4.78 (2 H, dd, J = 8.88, 6.47 Hz), 4.26 (2 H, br d, J = 8.99 Hz), | | |
| | 2.30 (3 H, s) | | |
| 177 | 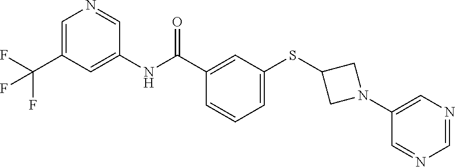 [SMILES: O=C(Nc1cncc(C(F)(F)F)c1)c1cccc(SC2CN(c3cncnc3)C2)c1] | 32 mg (44%) | RuPhos, Pd(dppf)Cl₂ Cs₂CO₃ DMA |
| | LC-MS (ESI, m/z): method 4, t_{R} = 6.76 min, m/z (M + 1) = 446.23 | | |
| | ¹H NMR (400 MHz, acetone-d₆) δ ppm 10.02 (1 H, br s), 9.16 (1 H, s), 8.74 (1 H, s), 8.67 (1 H, s), 8.54 (1 H, | | |
| | s), 8.07 (2 H, s), 7.83 (2 H, m), 7.43 (1 H, d, J = 8.33 Hz), 4.61 (3 H, m), 4.01 (2 H, dd, J = 8.00, 4.49 Hz), | | |
| | 2.43 (3 H, s) | | |
| 178 | 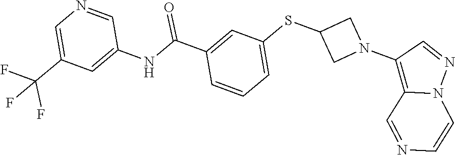 [SMILES: O=C(Nc1cncc(C(F)(F)F)c1)c1cccc(SC2CN(c3cnn4ccncc34)C2)c1] | 56 mg (42%) | tBuBrettPhos Pd G3 Cs₂CO₃ Me-THF |
| | LC-MS (ESI, m/z): method 4, t_{R} = 6.85 min, m/z (M + 1) = 485.20 | | |
| | ¹H NMR (400 MHz, acetone-d₆) δ ppm 10.02 (1 H, br s), 9.16 (1 H, s), 8.93 (1 H, d, J = 1.32 Hz), 8.75 (1 H, s), | | |
| | 8.67(1 H, s), 8.27 (1 H, dd, J = 5.04, 1.32 Hz), 7.85 (1 H, m), 7.83 (1 H, m), 7.62 (1 H, m), 7.58 (1 H, m), 7.42 | | |
| | (1 H, d, J = 7.89 Hz), 4.66 (2 H, m), 4.55 (1 H, m), 4.04 (2 H, dd, J = 7.56, 5.37 Hz), 2.43 (3 H, s) | | |
| 179 | 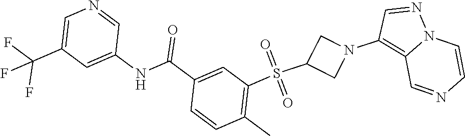 [SMILES: Cc1ccc(C(=O)Nc2cncc(C(F)(F)F)c2)cc1S(=O)(=O)C1CN(c2cnn3ccncc23)C1] | 33 mg (8%) | tBuBrettPhos Pd G3 Cs₂CO₃ Me-THF |
| | LC-MS (ESI, m/z): method 4, t_{R} = 5.63 min, m/z (M + 1) = 517.13 | | |
| | ¹H NMR (400 MHz, acetone-d₆) δ ppm 10.36 (1 H, br s), 9.14 (1 H, d, J = 1.97 Hz), 8.92 (1 H, d, J = 1.32 Hz), | | |
| | 8.71 (2 H, br d, J = 17.76 Hz), 8.62 (1 H, d, J = 1.97 Hz), 8.30 (2 H, m), 7.71 (1 H, d, J = 7.89 Hz), 7.63 (2 H, | | |
| | m), 4.82 (1 H, m), 4.37 (4 H, m), 2.82 (3 H, s) | | |
| 184 | 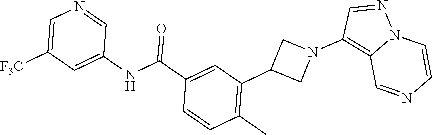 [SMILES: Cc1ccc(C(=O)Nc2cncc(C(F)(F)F)c2)cc1C1CN(c2cnn3ccncc23)C1] | | tBuBrettPhos Pd G3 Cs₂CO₃ DMA |
| | LC-MS (ESI, m/z): method 4, t_{R} = 6.27 min, m/z (M + 1) = 453.23 | | |
| | ¹H NMR (400 MHz, acetone) δ ppm 10.03 (1 H, m), 9.12 (1 H, s), 8.98 (1 H, s), 8.70 (1 H, s), 8.64 (1 H, s), | | |
| | 8.28 (1 H, dd, J = 4.93, 1.21 Hz), 8.13 (1 H, s), 7.85 (1 H, dd, J = 7.89, 1.53 Hz), 7.61 (2 H, m), 7.38 (1 H, | | |
| | d, J = 7.89 Hz), 4.60 (2 H, m), 4.39 (1 H, t, J = 7.56 Hz), 4.14 (2 H, m), 2.39 (3 H, s) | | |
| Inter- mediate 121 | 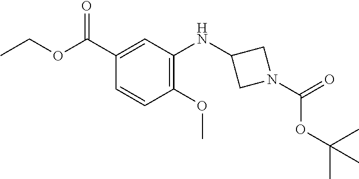 [SMILES: CCOC(=O)c1ccc(OC)c(NC2CN(C(=O)OC(C)(C)C)C2)c1] | 2.7 g (100%) | Pd-170 Cs₂CO₃ DMA |
| | LC-MS (ESI, m/z): method 13, t_{R} = 2.21 min, m/z (M + 1 − tButyl) = 294.94 | | |
| Inter- mediate 122 | 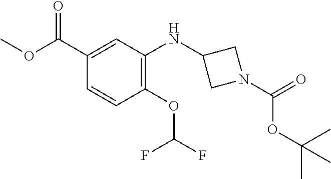 [SMILES: COC(=O)c1ccc(OC(F)F)c(NC2CN(C(=O)OC(C)(C)C)C2)c1] | 2.3 g (89%) | Pd-170 Cs₂CO₃ DMA |
| | LC-MS (ESI, m/z): method 13, t_{R} = 2.17 min, m/z (M + 1 − tButyl) = 316.9 | | |
| Inter- mediate 123 | 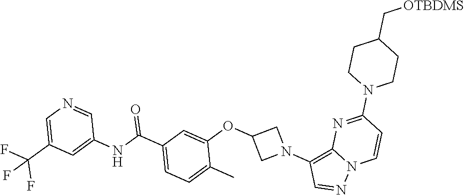 [SMILES: Cc1ccc(C(=O)Nc2cncc(C(F)(F)F)c2)cc1OC1CN(c2cnn3ccc(N4CCC(CO[Si](C)(C)C(C)(C)C)CC4)nc23)C1] | 30 mg (22%) | tBuBrettPhos Pd G3 Cs₂CO₃ Me-THF |
| | LC-MS (ESI, m/z): method 13, t_{R} = 3.06 min, m/z (M + 1) = 696.15 | | |
| Inter- mediate 124 | 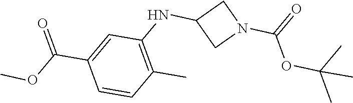 [SMILES: COC(=O)c1ccc(C)c(NC2CN(C(=O)OC(C)(C)C)C2)c1] | 6.7 g (76%) | Pd-170, Pd(crotyl) (XPhos)CI Cs₂CO₃ DMA |
| | LC-MS (ESI, m/z): method 13, t_{R} = 2.14 min, m/z (M − tBu) = 265.0 | | |
| Inter- mediate 125 | 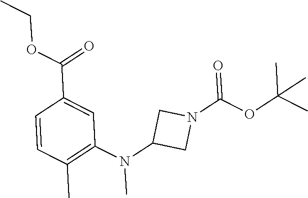 [SMILES: CCOC(=O)c1ccc(C)c(N(C)C2CN(C(=O)OC(C)(C)C)C2)c1] | 1.2 g (64%) | Pd 170 Cs₂CO₃ DMA |
| | LC-MS (ESI): method 13, t_{R} = 2.59 min, m/z (M − tBu) = 293.2 | | |
| Inter- mediate 126 | 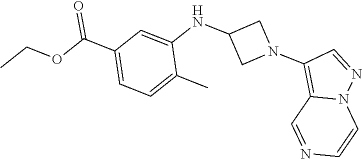 | 13 mg (33%) | BPC-305 tBuONa Me-THF |
| | LC-MS (ESI, m/z): method 12, t_{R} = 0.94 min, m/z (M + 1) = 352.17 | | |
| Inter- mediate 127 | 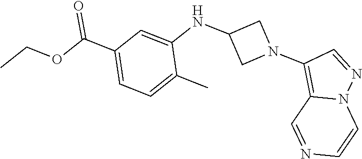 | 182 mg (48%) | Pd-175 Cs₂CO₃ Me-THF |
| | LC-MS (ESI, m/z): method 13, t_{R} = 1.87 min, m/z (M + 1) = 353.21 | | |
| Inter- mediate 128 | 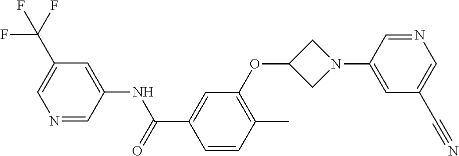 [SMILES: Cc1ccc(C(=O)Nc2cncc(C(F)(F)F)c2)cc1OC1CN(c2cncc(C#N)c2)C1] | 129 mg (100%) | RuPhos, Pd(dppf)Cl₂ Cs₂CO₃ DMA |

LC-MS (ESI, m/z): method 16, t_{R} = 2.40 min, m/z (M + 1) = 453.40

### Intermediate 129—tert-butyl 3-(5-(ethoxycarbonyl)-2-methylbenzyl)azetidine-1-carboxylate

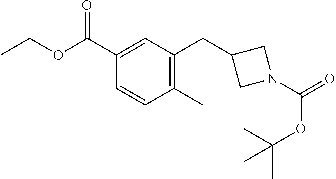 [SMILES: CCOC(=O)c1ccc(C)c(CC2CN(C(=O)OC(C)(C)C)C2)c1]

To an oven dried 10 mL ElectraSyn 2.0 vial, redox active ester tert-butyl 3-(2-((1,3-dioxoisoindolin-2-yl)oxy)-2-oxoethyl)azetidine-1-carboxylate (500 mg, 1.39 mmol), ethyl 3-bromo-4-methylbenzoate (506 mg, 2.081 mmol), dibromonickel 1-methoxy-2-(2-methoxyethoxy)ethane (98 mg, 0.28 mmol), and 2,2′-bipyridine (43.3 mg, 0.28 mmol) were all directly added as solids/oils. Anhydrous NMP (8 ml) was then added and the contents of the vial were allowed to stir until all solids were dissolved. Silver nitrate (118 mg, 0.69 mmol) was then added to the reaction mixture directly as a solid. The vial was closed with an ElectraSyn 2.0 vial cap with a (+) magnesium sacrificial anode (WE) and a 100 ppi (−) graphite cathode (3 mm×7 mm×51 mm) (CE). The vial was then placed on an IKA ElectraSyn 2.0 stir plate and electrolysis was set to 12 mA, 1.37 mmol, 3.0 F/mol. Electrodes were washed with ACN, then water were added with a solid precipitation. The solution was filtered to a celite pad and the resulting clear solution was extracting with MTBE. The organic layers were collected and dried over Na₂SO₄. The solvent was removed under vacuum and the crude was purified by reverse phase FCC (Eluent A/Eluent B: from 100:0 to 0:100) affording the title compound (137 mg, 0.41 mmol, 30%). LC-MS (ESI, m/z): method 13, t_{R}=2.49 min, m/z (M+1-BOC)=234.06

The following Intermediate was prepared using the above procedure, by starting from related Intermediate.

| | | Product Amount |
|---|---|---|
| Intermediate | Structure | (Yield) |
| Nº | Analytical Data | |
| Intermediate 130 | 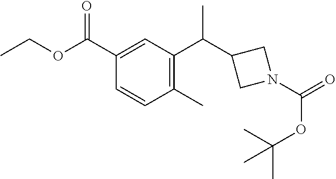 [SMILES: CCOC(=O)c1ccc(C)c(C(C)C2CN(C(=O)OC(C)(C)C)C2)c1] | 237 mg (17%) |
| | LC-MS (ESI, m/z): method 12, t_{R} = 1.43 min, m/z (M + 1 − BOC) = 248.22 | |
| Intermediate 136 | 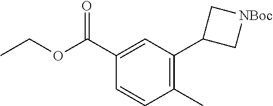 [SMILES: CCOC(=O)c1ccc(C)c(C2CN(C(=O)OC(C)(C)C)C2)c1] | 290 mg (21%) |

LC-MS (ESI, m/z): method 12, t_{R} = 1.33 min, m/z (M + 1 − BOC) = 220.24

### Intermediate 131—tert-butyl 3-((5-(ethoxycarbonyl)-2-methylphenyl)thio)azetidine-1-carboxylate

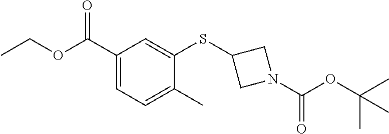 [SMILES: CCOC(=O)c1ccc(C)c(SC2CN(C(=O)OC(C)(C)C)C2)c1]

Ethyl 3-mercapto-4-methylbenzoate (715 mg, 3.64 mmol) and tert-butyl 3-hydroxyazetidine-1-carboxylate (820 mg, 4.74 mmol) were dissolved in toluene (12 ml), then 2-(tributyl-lambda5-phosphanylidene)acetonitrile (1.43 ml, 5.46 mmol) was added in one portion and the solution was purged with Ar for 2 min. The reaction mixture was stirred at 70° C. overnight. The mixture was evaporated under reduced pressure and the residue was dissolved in DCM and washed with NaHCO₃ and brine, the organic layer was dried over MgSO₄. The crude was purified via FCC (Eluent A: n-Heptane Eluent B: ethyl acetate, gradient A:B from 100:0 to 60:40). The relevant fractions were collected and desiccated until dryness to afford title compound (971.1 mg, 2.76 mmol, 76% yield).

LC-MS (ESI, m/z): method 13, t_{R}=2.64 min, m/z (M+1-tBytyl)=295.97

¹H NMR (acetone-d₆, 400 MHz) δ 7.76 (dd, 1H, J=1.4, 7.8 Hz), 7.66 (s, 1H), 7.37 (d, 1H, J=7.9 Hz), 4.46 (br s, 2H), 4.34 (q, 2H, J=7.0 Hz), 4.2-4.3 (m, 1H), 3.80 (br s, 2H), 2.39 (s, 3H), 1.42 (s, 9H), 1.36 (t, 3H, J=7.1 Hz)

The following Intermediate was prepared using the same procedure.

| | | Product Amount |
|---|---|---|
| Intermediate | Structure | (Yield) |
| Nº | Analytical Data | |
| 132 | 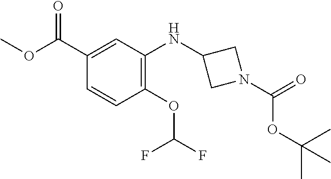 [SMILES: COC(=O)c1ccc(OC(F)F)c(NC2CN(C(=O)OC(C)(C)C)C2)c1] | 826 mg (97%) |

LC-MS (ESI, m/z): method 13, t_{R} = 2.22 min, m/z (M + 1 − tButyl) = 317

### Example 173—3-((1-(5-cyano-7H-pyrrolo[2,3-d]pyrimidin-4-yl)azetidin-3-yl)oxy)-4-methyl-N-(5-(trifluoromethyl)pyridin-3-yl)benzamide

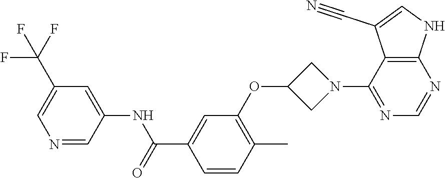 [SMILES: Cc1ccc(C(=O)Nc2cncc(C(F)(F)F)c2)cc1OC1CN(c2ncnc3[nH]cc(C#N)c23)C1]

Intermediate 111 (80 mg, 0.227 mmol) and 4-chloro-7H-pyrrolo[2,3-d]pyrimidine-5-carbonitrile (61 mg, 0.34 mmol) were loaded in a microwave vial and dissolved in ACN (3 mL). DIPEA (0.12 mL, 0.68 mmol) was added and the mixture was heated at 85° C. until complete conversion. The volatiles were removed under reduced pressure and the residue was partitioned between DCM and water. The organic layer was washed with sat. NaHCO₃, dried over sodium sulfate, filtered and concentrated. The residue was purified by either silica gel flash chromatography in direct phase FCC (28 g Sfar-D amino column, gradient A:B from 100:0 to 0:10 in 12 CV eluent A: n-Heptane Eluent B: Acetone). Relevant fractions were collected and the solvent was evaporated to afford desired product (73 mg, 0.15 mmol, 65%).

LC-MS (ESI, m/z): method 4, t_{R}=6.57 min, m/z (M+1)=494.19

¹H NMR (400 MHz, DMSO-d₆) δ ppm 10.72 (1H, m), 9.19 (1H, br s), 8.69 (1H, br s), 8.65 (1H, s), 8.05 (1H, s), 7.77 (1H, s), 7.61 (1H, d, J=7.67 Hz), 7.39 (1H, br d, J=7.67 Hz), 7.31 (1H, s), 5.30 (1H, m), 4.75 (2H, dd, J=9.54, 6.47 Hz), 4.21 (2H, dd, J=9.76, 3.62 Hz), 2.29 (3H, m)

The following Intermediates were prepared by using the procedure described for the synthesis of Example 173, starting from the suitable Intermediate, using ACN or DMA as solvent.

| | | Product Amount |
|---|---|---|
| Intermediate | Structure | (Yield) |
| Nº | Analytical Data | |
| 133 | 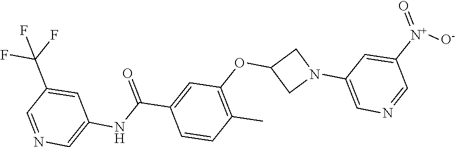 [SMILES: Cc1ccc(C(=O)Nc2cncc(C(F)(F)F)c2)cc1OC1CN(c2cncc([N+](=O)[O-])c2)C1] | 75 mg (37%) |
| | LC-MS (ESI, m/z): method 3 min, t_{R} = XX min, m/z (M + 1) = 474.2 | |
| 134 | 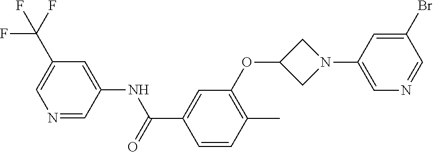 [SMILES: Cc1ccc(C(=O)Nc2cncc(C(F)(F)F)c2)cc1OC1CN(c2cncc(Br)c2)C1] | 25 mg (17%) |
| | LC-MS (ESI, m/z): method 2 min, t_{R} = 1.18 min, m/z (M + 1) = 508.17 | |
| 140 | 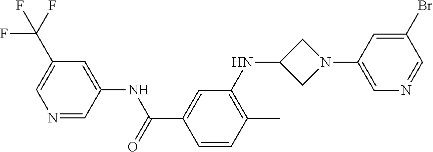 [SMILES: Cc1ccc(C(=O)Nc2cncc(C(F)(F)F)c2)cc1NC1CN(c2cncc(Br)c2)C1] | 32 mg (22%) |

LC-MS (ESI): Method 12, t_{R} = 1.18 min, m/z (M + 1) = 506.118

### Intermediate 139—3-((1-(5-aminopyridin-3-yl)azetidin-3-yl)oxy)-4-methyl-N-(5-(trifluoromethyl)pyridin-3-yl)benzamide

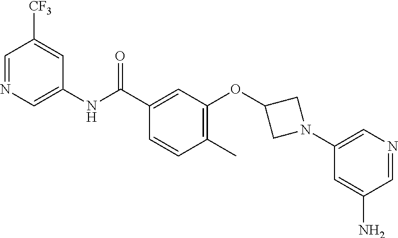 [SMILES: Cc1ccc(C(=O)Nc2cncc(C(F)(F)F)c2)cc1OC1CN(c2cncc(N)c2)C1]

In a 50 mL steel reactor vessel, Intermediate 133 (75 mg, 0.16 mmol), then JM Pd/C 10R424 (50% wet, 16.86 mg, 7.92 μmol) was added in one portion. The vessel was closed and 3 cycle vacuum-H₂ was performed to set the reaction. The reaction was run under 3 bar hydrogen (0.639 mg, 0.317 mmol) at 40° C., and stirred until completion. The mixture was filtered through celite pad then the solvent was removed by reduced pressure. The residue was purified by FCC (6 g Sfar-D amino column, gradient n-heptane:acetone from 100:0 to 0:100) to give the title compound (41 mug, 0.092 mmol, 58% o yield).

LC-MS (ESI): Method 4, tR=4.30 min, m/z (M+1)=444.2

### Example 139—5-(3-(2-methyl-5-((5-(trifluoromethyl)pyridin-3-yl)carbamoyl) phenoxy)azetidin-1-yl)nicotinamide

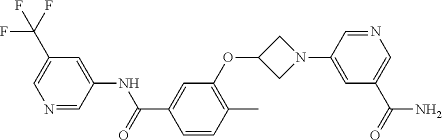 [SMILES: Cc1ccc(C(=O)Nc2cncc(C(F)(F)F)c2)cc1OC1CN(c2cncc(C(N)=O)c2)C1]

Intermediate 128 (0.150 g, 0.33 mmol) was dissolved in DMSO (1 mL), then K₂CO₃ (0.082 g, 0.595 mmol) was added. The suspension was stirred 5 min at rt, then H₂O₂ 30% (0.078 ml, 3.31 mmol) was added dropwise at 0° C. and the mixture was stirred overnight at rt. The reaction was quenched with H₂O, and the desired product started precipitating as a white solid. Solid was filtered and purified via prep HPLC (basic conditions) to obtain the title compound (40 mg, 0.085 mmol, 26%).

LC-MS (ESI): Method 10, t_{R}=2.80 min, m/z (M+1)=472.87

¹H NMR (400 MHz, DMSO-d₆) δ 10.69 (s, 1H), 9.19 (d, J=2.3 Hz, 1H), 8.69 (s, 1H), 8.64 (t, J=2.3 Hz, 1H), 8.40 (d, J=1.8 Hz, 1H), 8.02 (d, J=2.8 Hz, 2H), 7.62 (dd, J=7.7, 1.6 Hz, 1H), 7.49 (s, 1H), 7.40 (d, J=7.7 Hz, 1H), 7.31 (q, J=1.9 Hz, 2H), 5.33 (s, 1H), 4.49 (dd, J=8.7, 6.2 Hz, 2H), 3.95 (dd, J=8.9, 4.0 Hz, 2H), 2.28 (s, 3H).

### Example 143—4-methyl-3-((1-(5-(1-methyl-1H-pyrazol-4-yl)pyridin-3-yl)azetidin-3-yl)oxy)-N-(5-(trifluoromethyl)pyridin-3-yl)benzamide

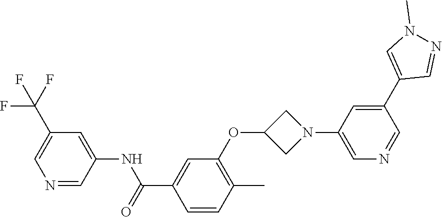 [SMILES: Cc1ccc(C(=O)Nc2cncc(C(F)(F)F)c2)cc1OC1CN(c2cncc(-c3cnn(C)c3)c2)C1]

A microwave vial was loaded with K₃PO₄ (43.9 mg, 0.21 mmol), Intermediate 134 (35 mg, 0.069 mmol), 1-methyl-4-(4,4,5,5-tetramethyl-1,3,2-dioxaborolan-2-yl)-1H-pyrazole (32 mg, 0.152 mmol), Pd-170, Pd(crotyl)(XPhos)CI (2.324 mg, 3.45 μmol), THF (2 mL) and water (2 mL). Solution was backfilled with Ar, then solution was stirred for 1 h at 50° C. The solvent was evaporated under vacuum. After purification by FCC (NH silica 6 gr, eluting with gradient 40% acetone in n-heptane) the title compound was obtained (16 mg, 0.03 mmol, 46%).

LC-MS (ESI): Method 4, t_{R}=5.08 min, m/z (M+1)=509.2

¹H NMR (DMSO-d₆, 400 MHz) δ 10.66 (s, 1H), 9.18 (d, 1H, J=2.2 Hz), 8.6-8.7 (m, 1H), 8.6-8.6 (m, 1H), 8.1-8.2 (m, 2H), 7.89 (s, 1H), 7.70 (d, 1H, J=2.6 Hz), 7.59 (dd, 1H, J=1.2, 7.8 Hz), 7.38 (d, 1H, J=7.9 Hz), 7.2-7.3 (m, 1H), 7.04 (t, 1H, J=2.2 Hz), 5.2-5.4 (m, 1H), 4.44 (dd, 2H, J=6.2, 8.4 Hz), 3.89 (dd, 2H, J=3.9, 8.8 Hz), 3.82 (s, 3H), 2.25 (s, 3H)

The following Example was prepared using the same procedure.

| | | Product Amount |
|---|---|---|
| Example | Structure | (Yield) |
| Nº | Analytical Data | |
| 144 | 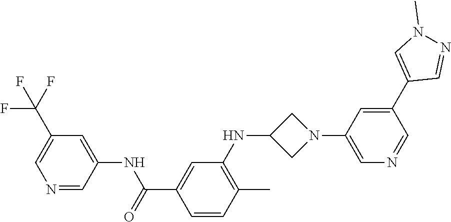 [SMILES: Cc1ccc(C(=O)Nc2cncc(C(F)(F)F)c2)cc1NC1CN(c2cncc(-c3cnn(C)c3)c2)C1] | 13 mg (52%) |

LC-MS (ESI): Method 4, t_{R} = 4.71 min, m/z (M + 1) = 508.21
¹H NMR (acetone-d₆, 400 MHz) δ 9.88 (br s, 1H), 9.17 (s, 1H), 8.75 (s, 1H), 8.64 (s, 1H), 8.17 (d,
1H, J = 1.8 Hz), 8.02 (s, 1H), 7.82 (s, 1H), 7.71 (d, 1H, J = 2.6 Hz), 7.34 (d, 1H, J = 7.4 Hz), 7.1-
7.2 (m, 2H), 7.01 (t, 1H, J = 2.2 Hz), 5.24 (br d, 1H, J = 7.1 Hz), 4.6-4.7 (m, 1H), 4.48 (t, 2H, J =
7.2 Hz), 3.89 (s, 3H), 3.8-3.9 (m, 2H), 2.26 (s, 3H)

### Example 145—3-((1-(5-acetamidopyridin-3-yl)azetidin-3-yl)oxy)-4-methyl-N-(5-(trifluoromethyl)pyridin-3-yl)benzamide

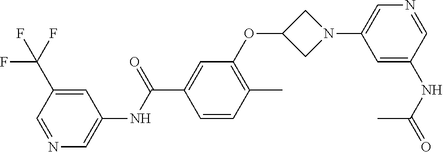 [SMILES: CC(=O)Nc1cncc(N2CC(Oc3cc(C(=O)Nc4cncc(C(F)(F)F)c4)ccc3C)C2)c1]

In a 50 mL steel reactor vessel, Intermediate 139 (32 mg, 0.072 mmol) was dissolved in pyridine (0.5 mL), then acetyl chloride (0.015 mL, 0.216 mmol) was added in one portion. The reaction was stirred at rt until completion. Solvent was removed by reduced pressure, and the crude was purified by FCC (Sfar-D amino column, gradient A:B from 100:0 to 0:100 in 12 CV eluent A: n-heptane, Eluent B: acetone) to afford the title compound (29 mg, 0.059 mmol, 82% yield).

LC-MS (ESI): Method 4, t_{R}=4.61 min, m/z (M+1)=486.22

¹H NMR (Acetone-d₆, 400 MHz) δ 9.98 (br s, 1H), 9.1-9.2 (m, 2H), 8.74 (s, 1H), 8.66 (s, 1H), 8.04 (d, 1H, J=1.8 Hz), 7.6-7.6 (m, 2H), 7.46 (br s, 1H), 7.3-7.4 (m, 2H), 5.3-5.4 (m, 1H), 4.5-4.5 (m, 2H), 3.96 (dd, 2H, J=4.1, 8.4 Hz), 2.32 (s, 3H), 2.09 (s, 3H).

The following Examples and Intermediates were prepared using the procedure described for the synthesis of Intermediate 104 by reacting the corresponding precursor with the suitable amine.

| | | Product Amount |
|---|---|---|
| Example Nº/ | Structure | (Yield) |
| Intermediate Nº | Analytical Data | |
| 149 | 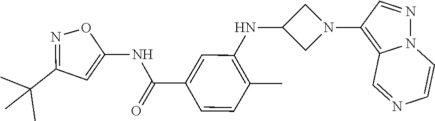 | 13 mg (25%) |
| | LC-MS (ESI, m/z): method 4, t_{R} = 5.74 min, m/z (M + 1) = 446.24 | |
| | ¹H NMR (DMSO-d₆, 400 MHz) δ 11.14 (s, 1H), 8.96 (d, 1H, J = 1.5 Hz), 8.42 (dd, 1H, J = 1.4, 4.9 | |
| | Hz), 7.62 (s, 1H), 7.58 (d, 1H, J = 4.8 Hz), 7.25 (dd, 1H, J = 1.4, 7.6 Hz), 7.1-7.1 (m, 2H), 6.71 (s, | |
| | 1H), 5.59 (d, 1H, J = 7.0 Hz), 4.57 (br d, 1H, J = 6.6 Hz), 4.4-4.5 (m, 2H), 3.84 (t, 2H, J = 6.5 Hz), | |
| | 2.14 (s, 3H), 1.29 (s, 9H) | |
| 150 | 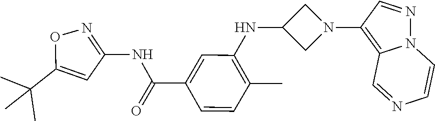 | 21 mg (46%) |
| | LC-MS (ESI, m/z): method 4, t_{R} = 5.74 min, m/z (M + 1) = 446.25 | |
| | ¹H NMR (ACN-d₃, 400 MHz) δ 9.22 (br s, 1H), 8.88 (d, 1H, J = 1.1 Hz), 8.17 (dd, 1H, J = 1.3, 5.1 | |
| | Hz), 7.58 (d, 1H, J = 4.9 Hz), 7.51 (s, 1H), 7.24 (dd, 1H, J = 1.3, 7.7 Hz), 7.18 (d, 1H, J = 7.7 Hz), | |
| | 7.01 (s, 1H), 6.75 (s, 1H), 6.67 (s, 2 H), 4.6-4.7 (m, 2H), 4.5-4.6 (m, 2H), 3.88 (t, 2H, J = 6.2 Hz), | |
| | 2.21 (s, 3H), 1.37 (s, 9H) | |
| 152 | 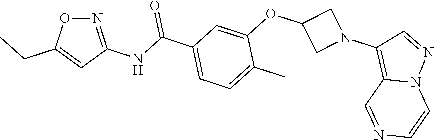 [SMILES: CCc1cc(NC(=O)c2ccc(C)c(OC3CN(c4cnn5ccncc45)C3)c2)no1] | 40 mg (67%) |
| | LC-MS (ESI, m/z): method 4, t_{R} = 3.86 min, m/z (M + 1) = 419.26 | |
| | ¹H NMR (DMSO-d₆, 400 MHz) δ 11.27 (s, 1H), 8.98 (d, 1H, J = 1.5 Hz), 8.43 (dd, 1H, J = 1.4, 4.9 | |
| | Hz), 7.66 (s, 1H), 7.60 (d, 1H, J = 4.8 Hz), 7.56 (dd, 1H, J = 1.3, 7.7 Hz), 7.36 (s, 1H), 7.30 (d, 1H, | |
| | J = 8.1 Hz), 6.75 (s, 1H), 5.30 (br t, 1H, J = 4.6 Hz), 4.52 (dd, 2H, J = 6.8, 7.7 Hz), 3.95 (dd, 2H, | |
| | J = 4.4, 8.3 Hz), 2.74 (q, 2H, J = 7.6 Hz), 2.22 (s, 3H), 1.21 (t, 3H, J = 7.6 Hz) | |
| 153 | 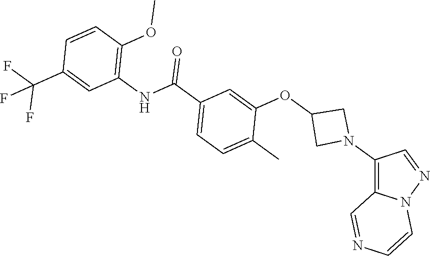 [SMILES: COc1ccc(C(F)(F)F)cc1NC(=O)c1ccc(C)c(OC2CN(c3cnn4ccncc34)C2)c1] | 21 mg (53%) |
| | LC-MS (ESI, m/z): method 4, t_{R} = 5.19 min, m/z (M + 1) = 498.21 | |
| | ¹H NMR (DMSO-d₆, 400 MHz) δ 9.57 (s, 1H), 8.98 (s, 1H), 8.43 (dd, 1H, J = 1.4, 4.9 Hz), 8.14 (d, | |
| | 1H, J = 2.2 Hz), 7.66 (s, 1H), 7.60 (d, 1H, J = 4.8 Hz), 7.52 (t, 2H, J = 9.3 Hz), 7.2-7.3 (m, 3H), 5.29 | |
| | (br t, 1H, J = 4.6 Hz), 4.5-4.5 (m, 2H), 3.97 (dd, 2H, J = 4.4, 8.3 Hz), 3.91 (s, 3H), 2.23 (s, 3H) | |
| 154 | 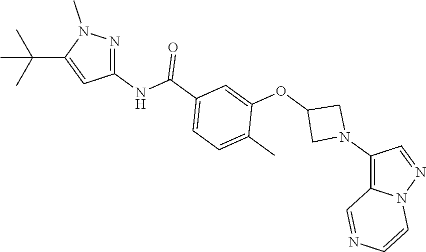 [SMILES: Cc1ccc(C(=O)Nc2cc(C(C)(C)C)n(C)n2)cc1OC1CN(c2cnn3ccncc23)C1] | 29 mg (44%) |
| | LC-MS (ESI, m/z): method 4, t_{R} = 6.56 min, m/z (M + 1) = 460.32 | |
| | ¹H NMR (DMSO-d₆, 400 MHz) δ 10.67 (s, 1H), 8.98 (d, 1H, J = 1.1 Hz), 8.43 (dd, 1H, J = 1.2, 4.9 | |
| | Hz), 7.66 (s, 1H), 7.59 (d, 1H, J = 5.0 Hz), 7.52 (d, 1H, J = 8.3 Hz), 7.33 (s, 1H), 7.25 (d, 1H, J = | |
| | 7.7 Hz), 6.45 (s, 1H), 5.30 (br t, 1H, J = 4.9 Hz), 4.52 (t, 2H, J = 7.1 Hz), 3.95 (dd, 2H, J = 4.4, 8.1 | |
| | Hz), 3.82 (s, 3H), 2.20 (s, 3H), 1.32 (s, 9H) | |
| 155 | 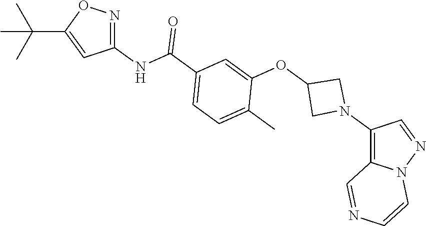 [SMILES: Cc1ccc(C(=O)Nc2cc(C(C)(C)C)on2)cc1OC1CN(c2cnn3ccncc23)C1] | 27 mg (43%) |
| | LC-MS (ESI, m/z): method 4, t_{R} = 4.64 min, m/z (M + 1) = 447.29 | |
| | ¹H NMR (400 MHz, DMSO-d₆) δ ppm 11.32 (1 H, s), 9.02 (1 H, d, J = 1.53 Hz), 8.47 (1 H, dd, J = | |
| | 5.04, 1.32 Hz), 7.70 (1 H, s), 7.63 (1 H, d, J = 5.04 Hz), 7.59 (1 H, dd, J = 7.89, 1.32 Hz), 7.39 (1 H, | |
| | s), 7.34 (1 H, d, J = 8.11 Hz), 6.75 (1 H, s), 5.33 (1 H, s), 4.55 (2 H, m), 3.99 (2 H, dd, J = 8.33, 4.38 | |
| | Hz), 2.25 (3 H, s), 1.33 (9 H, s) | |
| 156 | 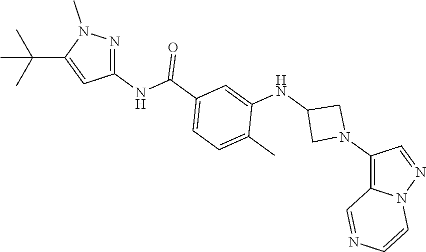 [SMILES: Cc1ccc(C(=O)Nc2cc(C(C)(C)C)n(C)n2)cc1NC1CN(c2cnn3ccncc23)C1] | 12 mg (18%) |
| | LC-MS (ESI, m/z): method 4, t_{R} = 5.90 min, m/z (M + 1) = 459.30 | |
| | ¹H NMR (400 MHz, DMSO-d₆) δ ppm 10.56 (1 H, s), 8.99 (1 H, d, J = 1.10 Hz), 8.45 (1 H, dd, J = | |
| | 4.82, 1.10 Hz), 7.65 (1 H, s), 7.61 (1 H, d, J = 4.82 Hz), 7.24 (1 H, d, J = 7.23 Hz), 7.09 (2 H, m), | |
| | 6.48 (1 H, s), 5.55 (1 H, d, J = 7.45 Hz), 4.61 (1 H, m), 4.50 (2 H, t, J = 6.80 Hz), 3.87 (2 H, m), | |
| | 3.84 (3 H, s), 2.16 (3 H, s), 1.35 (9 H, s) | |
| 157 | 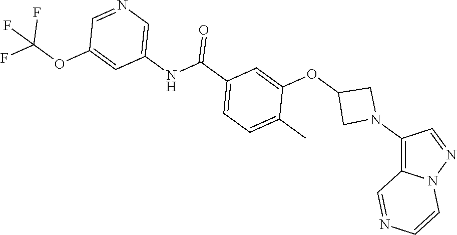 [SMILES: Cc1ccc(C(=O)Nc2cncc(OC(F)(F)F)c2)cc1OC1CN(c2cnn3ccncc23)C1] | 25 mg (37%) |
| | LC-MS (ESI, m/z): method 4, t_{R} = 6.85 min, m/z (M + 1) = 485.19 | |
| | ¹H NMR (400 MHz, DMSO-d₆) δ ppm 10.66 (1 H, s), 9.02 (1 H, d, J = 1.32 Hz), 8.97 (1 H, d, J = | |
| | 1.97 Hz), 8.45 (3 H, m), 7.70 (1 H, s), 7.63 (1 H, d, J = 4.82 Hz), 7.60 (1 H, dd, J = 7.67, 1.32 Hz), | |
| | 7.40 (1 H, d, J = 7.89 Hz), 7.29 (1 H, s), 5.32 (1 H, s), 4.56 (2 H, m), 4.00 (2 H, dd, J = 8.33, 4.38 | |
| | Hz), 2.28 (3 H, s) | |
| 158 | 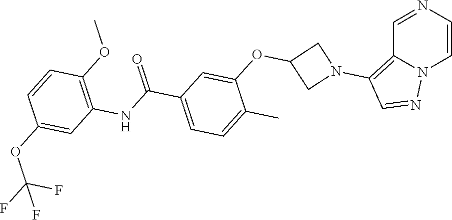 [SMILES: COc1ccc(OC(F)(F)F)cc1NC(=O)c1ccc(C)c(OC2CN(c3cnn4ccncc34)C2)c1] | 36 mg (25%) |
| | LC-MS (ESI, m/z): method 4, t_{R} = 5.4 min, m/z (M + 1) = 514.26 | |
| | ¹H NMR (400 MHz, DMSO-d₆) δ ppm 9.51 (1 H, s), 9.02 (1 H, d, J = 1.32 Hz), 8.47 (1 H, dd, J = | |
| | 5.04, 1.32 Hz), 7.92 (1 H, s), 7.69 (1 H, s), 7.63 (1 H, d, J = 4.82 Hz), 7.53 (1 H, d, J = 7.38 Hz), | |
| | 7.35 (1 H, d, J = 7.89 Hz), 7.28 (1 H, s), 7.20 (2 H, s), 5.32 (1 H, br t, J = 5.04 Hz), 4.54 (2 H, t, | |
| | J = 7.23 Hz), 4.01 (2 H, dd, J = 8.33, 4.38 Hz), 3.90 (3 H, s), 2.27 (3 H, s) | |
| 159 | 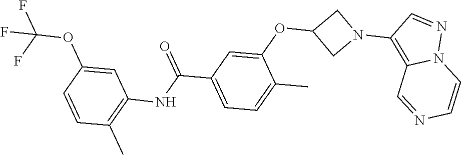 [SMILES: Cc1ccc(OC(F)(F)F)cc1NC(=O)c1ccc(C)c(OC2CN(c3cnn4ccncc34)C2)c1] | 82 mg (37%) |
| | LC-MS (ESI, m/z): method 11, t_{R} = 2.72 min, m/z (M + 1) = 498.22 | |
| | ¹H NMR (400 MHz, DMSO-d₆) δ 9.90 (s, 1H), 9.01 (d, J = 1.4 Hz, 1H), 8.47 (dd, J = 4.9, 1.5 Hz, | |
| | 1H), 7.69 (s, 1H), 7.63 (d, J = 4.9 Hz, 1H), 7.57 (dd, J = 7.8, 1.6 Hz, 1H), 7.48 (s, 1H), 7.41 (d, J = | |
| | 8.5 Hz, 1H), 7.36 (d, J = 7.8 Hz, 1H), 7.29 (d, J = 1.7 Hz, 1H), 7.18 (d, J = 8.9 Hz, 1H), 5.31 (h, J = | |
| | 4.9, 4.2 Hz, 1H), 4.62-4.49 (m, 2H), 4.00 (dd, J = 8.3, 4.4 Hz, 2H), 2.29 (s, 3H), 2.27 (s, 3H). | |
| 160 | 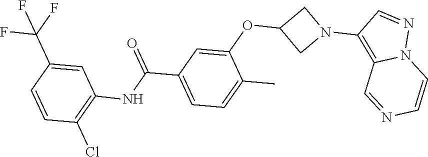 [SMILES: Cc1ccc(C(=O)Nc2cc(C(F)(F)F)ccc2Cl)cc1OC1CN(c2cnn3ccncc23)C1] | 82 mg (37%) |
| | LC-MS (ESI, m/z): method 15, t_{R} = 2.54 min, m/z (M + 1) = 502.11 | |
| | ¹H NMR (400 MHz, DMSO-d₆) δ 10.19 (s, 1H), 9.01 (d, J = 1.4 Hz, 1H), 8.47 (dd, J = 4.9, 1.4 Hz, | |
| | 1H), 8.05 (d, J = 2.3 Hz, 1H), 7.84 (d, J = 8.4 Hz, 1H), 7.71-7.65 (m, 2H), 7.63 (d, J = 4.9 Hz, 1H), | |
| | 7.59 (dd, J = 7.7, 1.6 Hz, 1H), 7.38 (d, J = 7.8 Hz, 1H), 7.33 (d, J = 1.7 Hz, 1H), 5.39-5.26 (m, 1H), | |
| | 4.55 (dd, J = 8.2, 6.0 Hz, 2H), 4.01 (dd, J = 8.3, 4.4 Hz, 2H), 2.28 (s, 3H). | |
| Intermediate 135 | 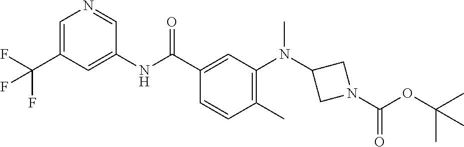 [SMILES: Cc1ccc(C(=O)Nc2cncc(C(F)(F)F)c2)cc1N(C)C1CN(C(=O)OC(C)(C)C)C1] | 1.310 g (82%) |

LC-MS (ESI): method 13, t_{R} = 2.47 min, m/z (M + 1) = 465.16

### Example 161—4-methyl-3-((1-(pyrazolo[1,5-a]pyrazin-3-yl)azetidin-3-yl)oxy)-N-(3-(trifluoromethoxy)phenyl)benzamide

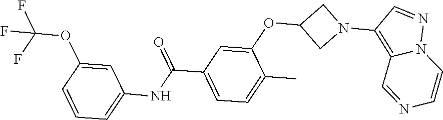 [SMILES: Cc1ccc(C(=O)Nc2cccc(OC(F)(F)F)c2)cc1OC1CN(c2cnn3ccncc23)C1]

Intermediate 127 (100 mg, 0.30 mmol) was placed in a flask, then DMF (0.86 mL) was added followed by DIPEA (0.529 mL, 3.0 mmol). After 5 min HATU (0.127 g, 0.33 mmol) was added and the solution was stirred at rt for 30 min. Next 3-(trifluoromethoxy)aniline (0.054 g, 0.30 mmol) was added and reaction mixture was stirred at rt overnight. The mixture was diluted with water and extracted with EtOAc. Organic layer was washed with water, brine, dried over Na₂SO₄ and concentrated on rotary evaporator. The crude material was purified via FCC (hexane/(3:1 AcOEt:iPrOH) from 100/0 to 60/40) and the title product was obtained (12 mg, 0.025 mmol, 8%).

LC-MS (ESI): Method 15, t_{R}=2.12 min, m/z (M+1)=483.93

¹H NMR (400 MHz, DMSO-d₆) δ 10.41 (s, 1H), 9.02 (d, J=1.4 Hz, 1H), 8.46 (dd, J=4.9, 1.4 Hz, 1H), 7.96 (d, J=2.6 Hz, 1H), 7.80-7.72 (m, 1H), 7.69 (s, 1H), 7.63 (d, J=4.9 Hz, 1H), 7.57 (dd, J=7.7, 1.6 Hz, 1H), 7.50 (t, J=8.2 Hz, 1H), 7.37 (d, J=7.8 Hz, 1H), 7.27 (d, J=1.6 Hz, 1H), 7.10 (dt, J=8.2, 1.4 Hz).

The following Examples were prepared using the same procedure, starting from the corresponding amine.

| | | Product Amount |
|---|---|---|
| Example | Structure | (Yield) |
| Nº | Analytical Data | |
| 162 | 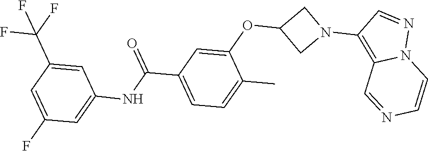 [SMILES: Cc1ccc(C(=O)Nc2cc(F)cc(C(F)(F)F)c2)cc1OC1CN(c2cnn3ccncc23)C1] | 22 mg (7%) |
| | LC-MS (ESI): Method 15, t_{R} = 2.36 min, m/z (M+1) = 486.08 | |
| | ¹H NMR (400 MHz, DMSO-d₆) δ 10.62 (s, 1H), 9.02 (d, J = 1.4 Hz, 1H), 8.47 (dd, J = 4.9, 1.5 Hz, | |
| | 1H), 8.08-7.97 (m, 2H), 7.70 (s, 1H), 7.63 (d, J = 4.9 Hz, 1H), 7.59 (dd, J = 7.7, 1.6 Hz, 1H), 7.38 | |
| | (d, J = 7.7 Hz, 2H), 7.29 (d, J = 1.6 Hz, 1H), 5.32 (t, J = 5.4 Hz, 1H), 4.63-4.49 (m, 2H), 4.00 (dd, | |
| | J = 8.3, 4.3 Hz, 2H), 2.27 (s, 3H). | |
| 163 | 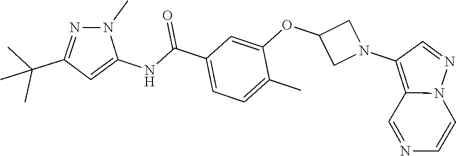 [SMILES: Cc1ccc(C(=O)Nc2cc(C(C)(C)C)nn2C)cc1OC1CN(c2cnn3ccncc23)C1] | 11 mg (14%) |
| | LC-MS (ESI): Method 4, t_{R} = 6.24 min, m/z (M+1) = 460.30 | |
| | ¹H NMR (400 MHz, DMSO-d₆) δ ppm 10.16 (1 H, s), 9.01 (1 H, d, J = 1.53 Hz), 8.46 (1 H, dd, J = | |
| | 4.82, 1.32 Hz), 7.69 (1 H, s), 7.63 (1 H, d, J = 5.04 Hz), 7.54 (1 H, m), 7.35 (1 H, d, J = 8.11 Hz), | |
| | 7.28 (1 H, s), 6.11 (1 H, s), 5.29 (1 H, br s), 4.55 (2 H, m), 4.00 (2 H, dd, J = 8.33, 4.38 Hz), 3.63 | |
| | (3 H, s), 2.26 (3 H, s), 1.24 (9 H, s) | |
| 164 | 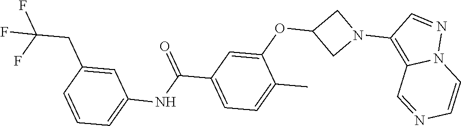 [SMILES: Cc1ccc(C(=O)Nc2cccc(CC(F)(F)F)c2)cc1OC1CN(c2cnn3ccncc23)C1] | 11 mg (8%) |

LC-MS (ESI): Method 15, t_{R} = 1.77 min, m/z (M+1) = 481.92
¹H NMR (400 MHz, DMSO-d₆) δ 10.22 (s, 1H), 9.02 (d, J = 1.5 Hz, 1H), 8.47 (dd, J = 4.9, 1.4 Hz,
1H), 7.83 (s, 1H), 7.77-7.72 (m, 1H), 7.70 (s, 1H), 7.63 (d, J = 4.9 Hz, 1H), 7.57 (dd, J = 7.6, 1.6
Hz, 1H), 7.42-7.33 (m, 2H), 7.28 (d, J = 1.7 Hz, 1H), 7.11 (d, J = 7.6 Hz, 1H), 5.38-5.28 (m, 1H),
4.62-4.53 (m, 2H), 4.01 (dd, J = 8.4, 4.4 Hz, 2H), 3.66 (q, J = 11.5 Hz, 2H), 2.27 (s, 3H).

### Example 175 and Example 176—4-methyl-3-(1-(1-(pyrazolo[1,5-a]pyrazin-3-yl)azetidin-3-yl)ethyl)-N-(5-(trifluoromethyl)pyridin-3-yl)benzamide

Racemic mixture of Example 172 (22 mg, 0.0458 mmol) was separated by chiral SFC (LUX Cellulose-1 10×250 mm, 5 μm 30/70 MeOH (0.1% NH₄OH)/CO₂, 15 mL/min, 120 bar, 40° C., DAD 230 nm) to give two isomers.

The ¹H NMR spectra of 1^{st} and 2^{nd} eluted isomers were superimposable with the ¹H NMR spectrum of the racemic mixture.

| | | Product Amount |
|---|---|---|
| Example | Structure | (Yield)/ |
| Nº | Analytical Data | |
| 175 | 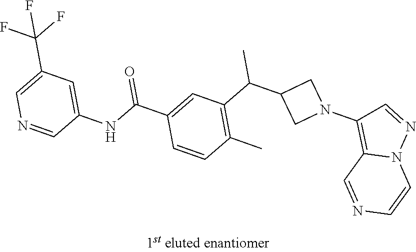 [SMILES: Cc1ccc(C(=O)Nc2cncc(C(F)(F)F)c2)cc1C(C)C1CN(c2cnn3ccncc23)C1] | 6.2 mg (28%) |
| | Chiral analysis (SFC Method 17): t_{R} 1.13 min, ee 100% | |
| | LC-MS (ESI, m/z): method 5, t_{R} = 4.50 min, m/z (M + 1) = 481.5 | |
| 176 | 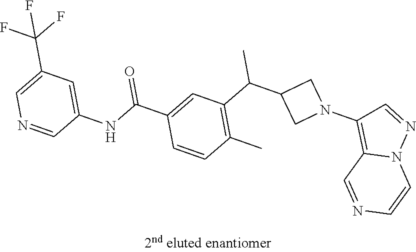 [SMILES: Cc1ccc(C(=O)Nc2cncc(C(F)(F)F)c2)cc1C(C)C1CN(c2cnn3ccncc23)C1] | 6.5 mg (29%) |

Chiral analysis (SFC Method 17): t_{R} 2.04 min, ee 98%
LC-MS (ESI, m/z): method 5, t_{R} = 4.50 min, m/z (M + 1) = 481.5

The following compounds of formula (Ic) were prepared by applying the experimental conditions described above:
4-methyl-3-((1-(pyrazin-2-yl)azetidin-3-yl)oxy)-N-(5-(trifluoromethyl)pyridin-3-yl)benzamide;
4-methyl-3-((1-(pyrazin-2-yl)azetidin-3-yl)amino)-N-(5-(trifluoromethyl)pyridin-3-yl)benzamide;
4-methyl-3-((1-(pyrazolo[1,5-a]pyrimidin-6-yl)azetidin-3-yl)oxy)-N-(5-(trifluoromethyl)pyridin-3-yl)benzamide;
4-methyl-3-((1-(5-(4-morpholinopiperidin-1-yl)pyrazolo[1,5-a]pyrimidin-3-yl)azetidin-3-yl)oxy)-N-(5-(trifluoromethyl)pyridin-3-yl)benzamide;
3-((1-(5-(4-methoxypiperidin-1-yl)pyrazolo[1,5-a]pyrimidin-3-yl)azetidin-3-yl)oxy)-4-methyl-N-(5-(trifluoromethyl)pyridin-3-yl)benzamide;
3-((1-(1H-pyrrolo[2,3-c]pyridin-4-yl)azetidin-3-yl)oxy)-4-methyl-N-(5-(trifluoromethyl)pyridin-3-yl)benzamide;
3-((1-(5-((2-methoxyethyl)amino)pyrazolo[1,5-a]pyrimidin-3-yl)azetidin-3-yl)oxy)-4-methyl-N-(5-(trifluoromethyl)pyridin-3-yl)benzamide;
3-((1-(furo[3,2-d]pyrimidin-4-yl)azetidin-3-yl)oxy)-4-methyl-N-(5-(trifluoromethyl)pyridin-3-yl)benzamide;
5-(3-((2-methyl-5-((5-(trifluoromethyl)pyridin-3-yl)carbamoyl)phenyl)amino)azetidin-1-yl)nicotinamide;
3-((1-(5-acetamidopyridin-3-yl)azetidin-3-yl)amino)-4-methyl-N-(5-(trifluoromethyl)pyridin-3-yl)benzamide;
3-((1-(5-aminopyridin-3-yl)azetidin-3-yl)oxy)-4-methyl-N-(5-(trifluoromethyl)pyridin-3-yl)benzamide;
3-((1-(5-aminopyridin-3-yl)azetidin-3-yl)amino)-4-methyl-N-(5-(trifluoromethyl)pyridin-3-yl)benzamide;
N-(5-ethylisoxazol-3-yl)-4-methyl-3-((1-(pyrazolo[1,5-a]pyrazin-3-yl)azetidin-3-yl)amino)benzamide;
N-(3-(tert-butyl)-1-methyl-1H-pyrazol-5-yl)-4-methyl-3-((1-(pyrazolo[1,5-a]pyrazin-3-yl)azetidin-3-yl)amino)benzamide;
N-(4-(difluoromethoxy)pyridin-2-yl)-4-methyl-3-((1-(pyrazolo[1,5-a]pyrazin-3-yl)azetidin-3-yl)amino)benzamide;
N-(3-cyclopropyl-1-methyl-1H-pyrazol-5-yl)-4-methyl-3-((1-(pyrazolo[1,5-a]pyrazin-3-yl)azetidin-3-yl)oxy)benzamide;
4-methyl-3-((1-(1-methyl-1H-indazol-7-yl)azetidin-3-yl)oxy)-N-(5-(trifluoromethyl)pyridin-3-yl)benzamide; and
4-methyl-3-((1-(pyrazolo[1,5-a]pyrazine-3-carbonyl)azetidin-3-yl)oxy)-N-(5-(trifluoromethyl)pyridin-3-yl)benzamide.

Comparative newly synthesized compound C1, characterized by having a —CO— linker between L and heteroaryl ring B, was prepared as following:

### Compound C1: 4-methyl-3-((1-(pyrazolo[1,5-a]pyrazine-3-carbonyl)azetidin-3-yl)amino)-N-(5-(trifluoromethyl)pyridin-3-yl)benzamide

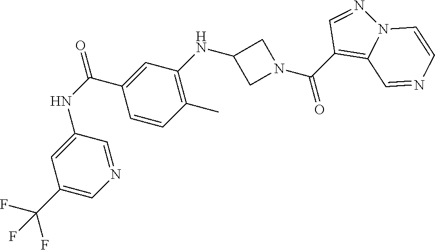 [SMILES: Cc1ccc(C(=O)Nc2cncc(C(F)(F)F)c2)cc1NC1CN(C(=O)c2cnn3ccncc23)C1]

Pyrazolo[1,5-a]pyrazine-3-carboxylic acid (0.05 g, 0.30 mmol) was placed into a flask, then DMF (0.82 mL) was added together with DIPEA (0.110 ml, 0.63 mmol). After 5 min HATU (0.119 g, 0.31 mmol) was added and the solution was stirred at rt for 30 min. Next Intermediate 115 (0.100 g, 0.285 mmol) was added and the reaction mixture was stirred at rt overnight. The it was diluted with water and extracted with DCM. Organic layer was washed with water, brine, dried over Na₂SO₄ and evaporated. The crude material was purified via FCC (eluting with DCM/MeOH from 100/0 to 90/10) and the title compound was obtained (51 mg, 0.103 mmol, 36%).

LC-MS (ESI, m/z): method 9, t_{R}=2.65 min, m/z (M+1)=496.16

¹H NMR (400 MHz, DMSO-d₆) δ 10.59 (s, 1H), 9.59 (d, J=1.4 Hz, 1H), 9.18 (s, 1H), 8.92 (dd, J=4.7, 1.5 Hz, 1H), 8.71-8.60 (m, 2H), 8.52 (s, 1H), 8.12 (d, J=4.7 Hz, 1H), 7.30 (dd, J=7.6, 1.7 Hz, 1H), 7.20 (d, J=7.7 Hz, 1H), 6.97 (d, J=1.7 Hz, 1H), 5.91-5.78 (m, 1H), 4.96 (s, 1H), 4.50 (s, 2H), 4.37 (s, 1H), 4.11 (s, 1H), 2.23 (s, 3H).

### Pharmacological Activity of the Compounds of the Invention

In vitro Assays

### Binding Assays

DDR1 and DDR2 binding assays were performed using Life Technologies LanthaScreen™ Europium Kinase Binding assay. The compounds were incubated with 5 nM DDR1 (Carna Biosciences) or 5 nM DDR2 (Life Technologies) for 1 h at rt in white 384-well OptiPlate (PerkinElmer), containing 20 nM or 10 nM Kinase Tracer 178 respectively and 2 nM Europium labelled anti-GST antibody (Life Technologies) in assay buffer (50 mM HEPES pH 7.5, 10 mM MgCI2, 1 mM EGTA and 0.01% BRIJ35). The ratio of fluorescence emission 665 nm/615 nm after excitation at 340 nm was obtained using the Tecan Spark 20M plate reader. IC₅₀ values were determined in GraphPad Prism 7.0 software, using 4 parameter model: log(inhibitor) vs. response. IC50 values were converted in Ki using the Cheng-Prusoff equation (Ki=IC50/(1+[Tracer]/Kd).

The results for individual compounds are provided herebelow in Table 2, wherein the compounds are classified in term of potency (nM) in binding with respect to their inhibitory activity on DDR1 and DDR2.

TABLE 2

| Example No | Ki DDR1 (nM) | Ki DDR2 (nM) |
|---|---|---|
| 134 | ++ | + |
| 135 | +++ | ++ |
| 136 | +++ | +++ |
| 137 | +++ | +++ |
| 138 | +++ | ++ |
| 139 | +++ | ++ |
| 140 | +++ | ++ |
| 141 | +++ | ++ |
| 142 | +++ | ++ |
| 143 | +++ | +++ |
| 144 | +++ | ++ |
| 145 | +++ | +++ |
| 146 | +++ | +++ |
| 147 | +++ | +++ |
| 148 | ++ | ++ |
| 149 | ++ | ++ |
| 150 | ++ | + |
| 151 | +++ | ++ |
| 152 | ++ | + |
| 153 | +++ | ++ |
| 154 | +++ | +++ |
| 155 | +++ | +++ |
| 156 | +++ | ++ |
| 157 | +++ | +++ |
| 158 | +++ | ++ |
| 159 | +++ | ++ |
| 160 | +++ | ++ |
| 161 | +++ | +++ |
| 162 | +++ | +++ |
| 163 | ++ | ++ |
| 164 | +++ | +++ |
| 165 | +++ | ++ |
| 166 | +++ | ++ |
| 167 | +++ | ++ |
| 168 | +++ | +++ |
| 169 | +++ | ++ |
| 170 | +++ | ++ |
| 171 | +++ | +++ |
| 172 | +++ | ++ |
| 173 | +++ | ++ |
| 174 | +++ | +++ |
| 175 | +++ | ++ |
| 176 | +++ | ++ |
| 177 | ++ | + |
| 178 | +++ | +++ |
| 179 | ++ | + |
| 180 | ++ | − |
| 181 | +++ | ++ |
| 182 | +++ | +++ |
| 183 | +++ | ++ |
| 184 | +++ | ++ |

+: Ki between 25 and 100 nM
++: Ki between 5 nM and 25 nM
+++: Ki lower than 5 nM
−: Ki higher than 100 nM

As it can be appreciated, the compounds of Table 2, i.e. compounds according to the invention, show a good activity as antagonists of DDR1 and DDR2. Accordingly, the compounds of the invention can be effectively used for treating diseases, disorders or conditions associated with DDR receptors, such as fibrosis, e.g. pulmonary fibrosis, idiopathic pulmonary fibrosis (IPF), hepatic fibrosis, renal fibrosis, ocular fibrosis, cardiac fibrosis, arterial fibrosis and systemic sclerosis.

### Comparative Examples

Compound C1 was tested in the same binding assay described above.

TABLE 3

| Example No | DDR1 Ki (nM) | DDR2 Ki (nM) |
|---|---|---|
| C1 | 114 | 311 |

The compounds of the present invention, as shown in Table 2, have a binding affinity for DDR1 and DDR2 receptors expressed as Ki lower than 100 nM, and, for most of the compounds, lower than 25 nM or even lower than 5 nM. To the contrary, comparative compound C1 has a binding affinity of 114 nM on DDR1 and higher than 300 nM on DDR2 receptor.

Thus, the presence of a direct link between L and heteroaryl ring B unexpectedly and noteworthy determines a remarkable increase in the inhibitory activity on the DDR1 and DDR2 receptors.

1: A compound of formula (I) 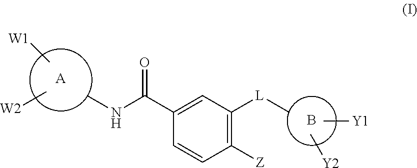
wherein
A is a ring selected from the group consisting of: 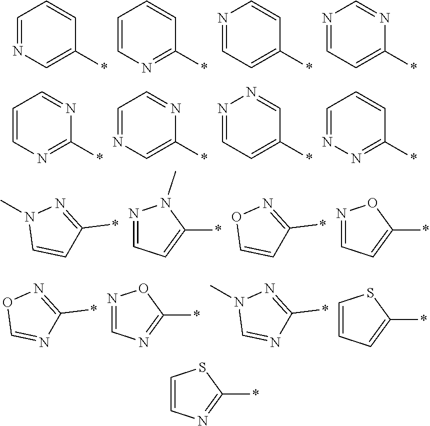
and phenyl, wherein [symbol] indicates a direct bond to NH;
W1 and W2 are substituents of ring A selected from the group consisting of hydrogen, halogen, (C₁-C₄)alkyl, (C₁-C₄)haloalkyl, (C₁-C₄)alkoxy, (C₁-C₄)haloalkoxy, R1R2N—(C₁-C₄)alkyl and (C₃-C₆)cycloalkyl;
Z is selected from the group consisting of (C₁-C₄)alkyl, (C₁-C₄)haloalkyl, (C₁-C₄)alkoxy, (C₁-C₄)haloalkoxy, (C₁-C₄)hydroxyalkyl and (C₃-C₆)cycloalkyl;
L is selected from the group consisting of 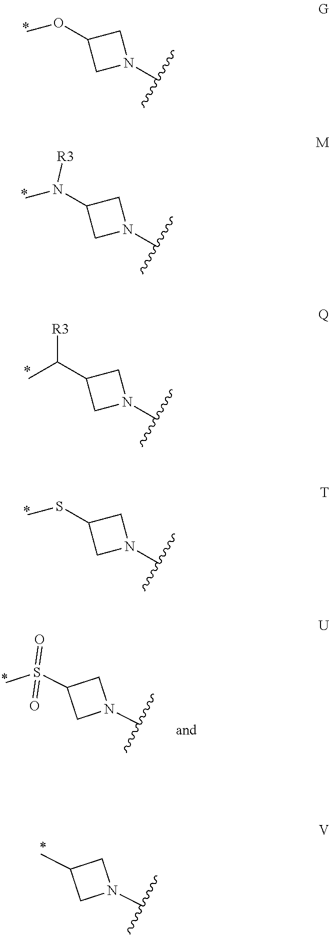
wherein [symbol] indicates a direct bond to the phenyl ring and 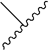
indicates a direct bond to B;
B is mono- or bi-cyclic heteroaryl ring;
Y1 and Y2 are substituents of ring B independently selected from the group consisting of hydrogen, (C₁-C₄)alkyl, (C₁-C₄)haloalkyl, (C₁-C₄)alkoxy, (C₁-C₄)alkoxy-(C₁-C₄)alkoxy, hydroxy-(C₁-C₄)alkoxy, hydroxy-(C₁-C₄)alkyl, (C₁-C₄)alkyl-heterocycloalkyl-(C₀-C₄)alkoxy, (C₁-C₄)haloalkoxy, halogen, cyano, cyano-(C₁-C₄)alkyl, cyano-(C₁-C₄)alkyl-heterocycloalkyl, CONR1R2, NHCOR1, NR1R2, R1R2N—(C₁-C₄)alkyl, heterocycloalkyl, (C₁-C₄)alkyl-heterocycloalkyl, heterocycloalkyl-(C₁-C₄)alkyl, (C₁-C₄)alkyl-heterocycloalkyl-carbonyl, (C₀-C₄)alkyl-heterocycloalkyl-carbonyl, (C₁-C₄)alkyl-phenyl and monocyclic (C₁-C₄)alkyl-heteroaryl;
R1 and R2 are independently selected from the group consisting of hydrogen, (C₁-C₄)alkyl, (C₁-C₄)hydroxyalkyl, (C₁-C₄)alkoxy(C₁-C₄)alkyl, (C₁-C₄)alkylamino-(C₁-C₄)alkyl, di-(C₁-C₄)alkylamino-(C₁-C₄)alkyl, optionally substituted (C₃-C₆)cycloalkyl, optionally substituted heterocycloalkyl and optionally substituted heterocycloalkyl-(C₁-C₄)alkoxy, wherein optional substituents are one or more and are selected from (C₁-C₄)alkyl, (C₁-C₄)alkoxy, (C₁-C₄)haloalkyl and (C₁-C₄)haloalkoxy; and
R3 is selected from the group consisting of hydrogen and (C₁-C₄)alkyl;
or a stereoisomer, tautomer, solvate or pharmaceutically acceptable salt thereof.

2: The compound, or stereoisomer, tautomer, solvate or pharmaceutically acceptable salt thereof, according to claim 1 wherein the compound has a formula (Ic) 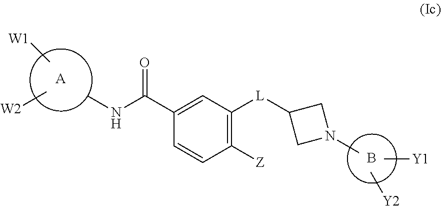
wherein L1 is selected from the group consisting of O, NR3, CHR3, S and SO₂, or is absent.

3: The compound, or stereoisomer, tautomer, solvate or pharmaceutically acceptable salt thereof, according to claim 1, wherein
B is selected from the group consisting of pyridinyl, pyrimidinyl, pyrazinyl, pyrazolo[1,5-a]pyrazinyl, 1H-pyrazolo[3,4-b]pyridinyl, pyrazolo[1,5-a]pyridinyl, pyrazolo[1,5-a]pyrimidinyl, imidazo[1,2-b]pyridazinyl, imidazo[1,2-a]pyrazinyl, imidazo[1,2-a]pyridinyl, thieno[3,2-d]pyrimidinyl, 1H-pyrrolo[2,3-b]pyridinyl and pyrazolyl.

4: The compound, or stereoisomer, tautomer, solvate or pharmaceutically acceptable salt thereof, according to claim 1, wherein
A is a ring selected from the group consisting of: 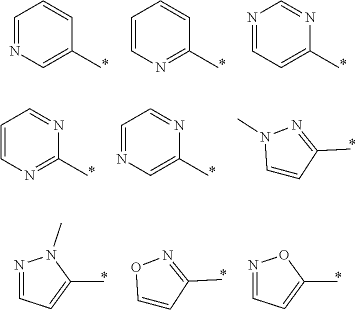
and phenyl.

5: The compound, or stereoisomer, tautomer, solvate or pharmaceutically acceptable salt thereof, according to claim 1, wherein
W1 and W2 are selected from the group consisting of H, F, CH₃, OCH₃, OCF₃, CF₃, C(CH₃)₃, CH₂CH₃, C(CH₃)₂CF₃, OCF₂H, CHF₂, CH₂CF₃, CH₂N(CH₃)₂ and cyclopropyl;
Z is selected from the group consisting of CH₃, CF₂H, OCH₃, CH₂OH and cyclopropyl;
L is selected from the group consisting of 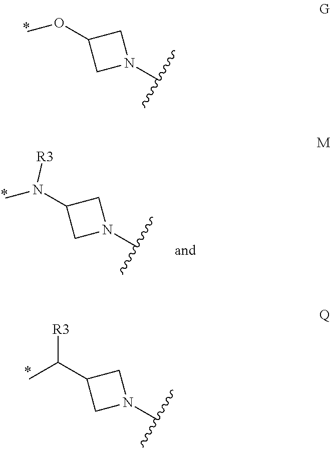
B is selected from the group consisting of pyridinyl, pyrimidinyl, pyrazinyl, pyrazolo[1,5-a]pyrazinyl, 1H-pyrazolo[3,4-b]pyridinyl, pyrazolo[1,5-a]pyridinyl, pyrazolo[1,5-a]pyrimidinyl, imidazo[1,2-b]pyridazinyl, imidazo[1,2-a]pyrazinyl, imidazo[1,2-a]pyridinyl, thieno[3,2-d]pyrimidinyl, 1H-pyrrolo[2,3-b]pyridinyl and pyrazolyl;
Y1 is selected from the group consisting of hydrogen, (C₁-C₄)haloalkyl, (C₁-C₄)alkoxy, cyano, CONR1R2, NHCOR1, NR1R2, (C₁-C₄)alkyl-heterocycloalkyl-carbonyl and monocyclic (C₁-C₄)alkyl-heteroaryl; and Y2 is hydrogen;
R1 and R2 are independently selected from the group consisting of CH₂CH₂N(CH₃)₂, CH₂CH₂OCH₃, CH₃, CH₂CH₂OH, oxetan-3-yl and 3-hydroxycyclobutyl; and
R3 is hydrogen or methyl.

6: The compound, or stereoisomer, tautomer, solvate or pharmaceutically acceptable salt thereof, according to claim 1, wherein the compound of formula (I) is selected from the group consisting of:
4-methyl-3-((1-(pyrimidin-5-yl)azetidin-3-yl)oxy)-N-(5-(trifluoromethyl) pyridin-3-yl)benzamide (Example 135);
4-methyl-3-((1-(pyrazolo[1,5-a]pyrazin-3-yl)azetidin-3-yl)oxy)-N-(5-(trifluoromethyl)pyridin-3-yl)benzamide (Example 136);
3-((1-(imidazo[1,2-a]pyridin-3-yl)azetidin-3-yl)oxy)-4-methyl-N-(5-(trifluoromethyl)pyridin-3-yl)benzamide (Example 137);
3-((1-(5-acetamidopyrazolo[1,5-a]pyrimidin-3-yl)azetidin-3-yl)oxy)-4-methyl-N-(5-(trifluoromethyl)pyridin-3-yl)benzamide (Example 138);
5-(3-(2-methyl-5-((5-(trifluoromethyl)pyridin-3-yl)carbamoyl)phenoxy)azetidin-1-yl)nicotinamide (Example 139);
4-methyl-3-((1-(5-(methylamino)pyrazolo[1,5-a]pyrimidin-3-yl)azetidin-3-yl)oxy)-N-(5-(trifluoromethyl)pyridin-3-yl)benzamide (Example 141);
4-methyl-3-((1-(pyrazolo[1,5-a]pyrimidin-3-yl)azetidin-3-yl)oxy)-N-(5-(trifluoromethyl)pyridin-3-yl)benzamide (Example 142);
4-methyl-3-((1-(5-(1-methyl-1H-pyrazol-4-yl)pyridin-3-yl)azetidin-3-yl)oxy)-N-(5-(trifluoromethyl)pyridin-3-yl)benzamide (Example 143);
3-((1-(5-acetamidopyridin-3-yl)azetidin-3-yl)oxy)-4-methyl-N-(5-(trifluoromethyl)pyridin-3-yl)benzamide (Example 145);
3-((1-(imidazo[1,2-a]pyrazin-3-yl)azetidin-3-yl)oxy)-4-methyl-N-(5-(trifluoromethyl)pyridin-3-yl)benzamide (Example 146);
N-(5-ethylisoxazol-3-yl)-4-methyl-3-((1-(pyrazolo[1,5-a]pyrazin-3-yl)azetidin-3-yl)oxy)benzamide (Example 152);
N-(2-methoxy-5-(trifluoromethyl)phenyl)-4-methyl-3-((1-(pyrazolo[1,5-a]pyrazin-3-yl)azetidin-3-yl)oxy)benzamide (Example 153);
N-(5-(tert-butyl)-1-methyl-1H-pyrazol-3-yl)-4-methyl-3-((1-(pyrazolo[1,5-a]pyrazin-3-yl)azetidin-3-yl)oxy)benzamide (Example 154);
N-(5-(tert-butyl)isoxazol-3-yl)-4-methyl-3-((1-(pyrazolo[1,5-a]pyrazin-3-yl)azetidin-3-yl)oxy)benzamide (Example 155);
4-methyl-3-((1-(pyrazolo[1,5-a]pyrazin-3-yl)azetidin-3-yl)oxy)-N-(5-(trifluoromethoxy)pyridin-3-yl)benzamide (Example 157);
N-(2-methoxy-5-(trifluoromethoxy)phenyl)-4-methyl-3-((1-(pyrazolo[1,5-a]pyrazin-3-yl)azetidin-3-yl)oxy)benzamide (Example 158);
4-methyl-N-(2-methyl-5-(trifluoromethoxy)phenyl)-3-((1-(pyrazolo[1,5-a]pyrazin-3-yl)azetidin-3-yl)oxy)benzamide (Example 159);
N-(2-chloro-5-(trifluoromethyl)phenyl)-4-methyl-3-((1-(pyrazolo[1,5-a]pyrazin-3-yl)azetidin-3-yl)oxy)benzamide (Example 160);
4-methyl-3-((1-(pyrazolo[1,5-a]pyrazin-3-yl)azetidin-3-yl)oxy)-N-(3-(trifluoromethoxy)phenyl)benzamide (Example 161);
N-(3-fluoro-5-(trifluoromethyl)phenyl)-4-methyl-3-((1-(pyrazolo[1,5-a]pyrazin-3-yl)azetidin-3-yl)oxy)benzamide (Example 162);
N-(3-(tert-butyl)-1-methyl-1H-pyrazol-5-yl)-4-methyl-3-((1-(pyrazolo[1,5-a]pyrazin-3-yl)azetidin-3-yl)oxy)benzamide (Example 163);
4-methyl-3-((1-(pyrazolo[1,5-a]pyrazin-3-yl)azetidin-3-yl)oxy)-N-(3-(2,2,2-trifluoroethyl)phenyl)benzamide (Example 164);
3-((1-(5-(difluoromethyl)pyridin-3-yl)azetidin-3-yl)oxy)-4-methyl-N-(5-(trifluoromethyl)pyridin-3-yl)benzamide (Example 165);
4-methyl-3-((1-(5-(4-methylpiperazin-1-yl)pyrazolo[1,5-a]pyrimidin-3-yl)azetidin-3-yl)oxy)-N-(5-(trifluoromethyl)pyridin-3-yl)benzamide (Example 166);
4-methyl-3-((1-(5-methylpyrazolo[1,5-a]pyrimidin-3-yl)azetidin-3-yl)oxy)-N-(5-(trifluoromethyl)pyridin-3-yl)benzamide (Example 167);
4-methyl-3-((1-(pyrazolo[1,5-a]pyridin-3-yl)azetidin-3-yl)oxy)-N-(5-(trifluoromethyl)pyridin-3-yl)benzamide (Example 168);
4-methyl-3-((1-(1-methyl-1H-pyrazolo[3,4-c]pyridin-4-yl)azetidin-3-yl)oxy)-N-(5-(trifluoromethyl)pyridin-3-yl)benzamide (Example 169);
4-methyl-3-((1-(5-morpholinopyrazolo[1,5-a]pyrimidin-3-yl)azetidin-3-yl)oxy)-N-(5-(trifluoromethyl)pyridin-3-yl)benzamide (Example 170);
3-((1-(5-cyano-7H-pyrrolo[2,3-d]pyrimidin-4-yl)azetidin-3-yl)oxy)-4-methyl-N-(5-(trifluoromethyl)pyridin-3-yl)benzamide (Example 173);
3-((1-(4-cyanopyridin-3-yl)azetidin-3-yl)oxy)-4-methyl-N-(5-(trifluoromethyl)pyridin-3-yl)benzamide (Example 174);
3-((1-(5-(4-(hydroxymethyl)piperidin-1-yl)pyrazolo[1,5-a]pyrimidin-3-yl)azetidin-3-yl)oxy)-4-methyl-N-(5-(trifluoromethyl)pyridin-3-yl)benzamide (Example 180);
4-(difluoromethoxy)-3-((1-(pyrazolo[1,5-a]pyrazin-3-yl)azetidin-3-yl)oxy)-N-(5-(trifluoromethyl)pyridin-3-yl)benzamide (Example 182);
4-methoxy-3-((1-(pyrazolo[1,5-a]pyrazin-3-yl)azetidin-3-yl)amino)-N-(5-(trifluoromethyl)pyridin-3-yl)benzamide (Example 183);
4-methyl-3-((1-(pyrimidin-5-yl)azetidin-3-yl)amino)-N-(5-(trifluoromethyl) pyridin-3-yl)benzamide (Example 134);
4-methyl-3-((1-(pyrazolo[1,5-a]pyrazin-3-yl)azetidin-3-yl)amino)-N-(5-(trifluoromethyl)pyridin-3-yl)benzamide (Example 140);
4-methyl-3-((1-(5-(1-methyl-1H-pyrazol-4-yl)pyridin-3-yl)azetidin-3-yl)amino)-N-(5-(trifluoromethyl)pyridin-3-yl)benzamide (Example 144);
3-((1-(imidazo[1,2-a]pyrazin-3-yl)azetidin-3-yl)amino)-4-methyl-N-(5-(trifluoromethyl)pyridin-3-yl)benzamide (Example 147);
4-methyl-3-((1-(pyrazolo[1,5-a]pyrimidin-3-yl)azetidin-3-yl)amino)-N-(5-(trifluoromethyl)pyridin-3-yl)benzamide (Example 148);
N-(3-(tert-butyl)isoxazol-5-yl)-4-methyl-3-((1-(pyrazolo[1,5-a]pyrazin-3-yl) azetidin-3-yl)amino)benzamide (Example 149);
N-(5-(tert-butyl)isoxazol-3-yl)-4-methyl-3-((1-(pyrazolo[1,5-a]pyrazin-3-yl) azetidin-3-yl)amino)benzamide (Example 150);
N-(5-(tert-butyl)-1-methyl-1H-pyrazol-3-yl)-4-methyl-3-((1-(pyrazolo[1,5-a]pyrazin-3-yl)azetidin-3-yl)amino)benzamide (Example 156);
4-(difluoromethoxy)-3-((1-(pyrazolo[1,5-a]pyrazin-3-yl)azetidin-3-yl)amino)-N-(5-(trifluoromethyl)pyridin-3-yl)benzamide (Example 181);
4-methyl-3-(methyl(1-(pyrazolo[1,5-a]pyrazin-3-yl)azetidin-3-yl)amino)-N-(5-(trifluoromethyl)pyridin-3-yl)benzamide (Example 151);
4-methyl-3-((1-(pyrazolo[1,5-a]pyrazin-3-yl)azetidin-3-yl)methyl)-N-(5-(trifluoromethyl)pyridin-3-yl)benzamide (Example 171);
4-methyl-3-(1-(1-(pyrazolo[1,5-a]pyrazin-3-yl)azetidin-3-yl)ethyl)-N-(5-(trifluoromethyl)pyridin-3-yl)benzamide (Example 172);
4-methyl-3-(1-(1-(pyrazolo[1,5-a]pyrazin-3-yl)azetidin-3-yl)ethyl)-N-(5-(trifluoromethyl)pyridin-3-yl)benzamide, 1^{st} eluted enantiomer (Example 175);
4-methyl-3-(1-(1-(pyrazolo[1,5-a]pyrazin-3-yl)azetidin-3-yl)ethyl)-N-(5-(trifluoromethyl)pyridin-3-yl)benzamide, 2^{nd} eluted enantiomer (Example 176);
4-methyl-3-((1-(pyrimidin-5-yl)azetidin-3-yl)thio)-N-(5-(trifluoromethyl)pyridin-3-yl)benzamide (Example 177);
4-methyl-3-((1-(pyrazolo[1,5-a]pyrazin-3-yl)azetidin-3-yl)thio)-N-(5-(trifluoromethyl)pyridin-3-yl)benzamide (Example 178);
4-methyl-3-((1-(pyrazolo[1,5-a]pyrazin-3-yl)azetidin-3-yl)sulfonyl)-N-(5-(trifluoromethyl)pyridin-3-yl)benzamide (Example 179); and
4-methyl-3-(1-(pyrazolo[1,5-a]pyrazin-3-yl)azetidin-3-yl)-N-(5-(trifluoromethyl)pyridin-3-yl)benzamide (Example 184).

7: A pharmaceutical composition comprising the compound, or stereoisomer, tautomer, solvate or pharmaceutically acceptable salt thereof, according to claim 1, in admixture with at least one pharmaceutically acceptable carrier and/or excipient.

8: The pharmaceutical composition according to claim 7, which is formulated for administration by inhalation.

9. (canceled)

10: A method of treating a disease, disorder or condition associated with dysregulation of DDR, comprising administering the pharmaceutical composition of claim 7 to a subject in need thereof.

11: A method of treating fibrosis and/or a disease, disorder or condition that involves fibrosis, comprising administering the pharmaceutical composition of claim 7 to a subject in need thereof.

12: The method according to claim 11, wherein the fibrosis is at least one selected from the group consisting of pulmonary fibrosis, idiopathic pulmonary fibrosis (IPF), hepatic fibrosis, renal fibrosis, ocular fibrosis, cardiac fibrosis, arterial fibrosis and systemic sclerosis.

13: The method according to claim 12, wherein the fibrosis comprises idiopathic pulmonary fibrosis (IPF).

14: An intermediate compound selected from the group consisting of a compound of formula (XXXIII) 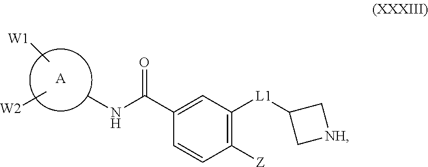
an intermediate compound of formula (XXXIX) 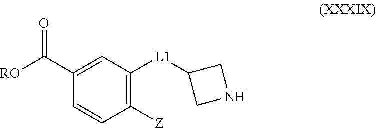
and an intermediate compound of formula (XL) 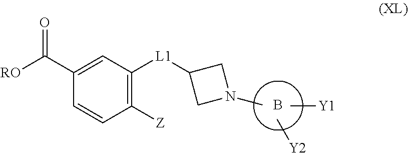
wherein
R is (C₁-C₄)alkyl,
A is a ring selected from the group consisting of: 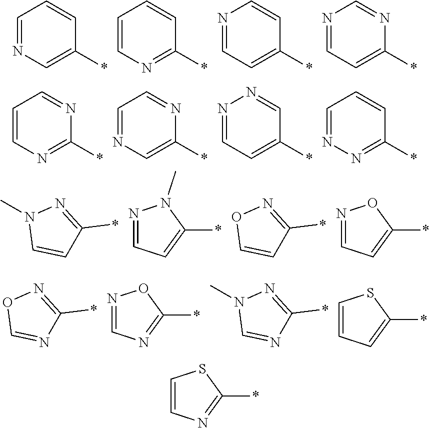
and phenyl, wherein [symbol] indicates a direct bond to NH;
W1 and W2 are substituents of ring A selected from the group consisting of hydrogen, halogen, (C₁-C₄)alkyl, (C₁-C₄)haloalkyl, (C₁-C₄)alkoxy, (C₁-C₄)haloalkoxy, R1R2N—(C₁-C₄)alkyl and (C₃-C₆)cycloalkyl;
Z is selected from the group consisting of (C₁-C₄)alkyl, (C₁-C₄)haloalkyl, (C₁-C₄)alkoxy, (C₁-C₄)haloalkoxy, (C₁-C₄)hydroxyalkyl and (C₃-C₆)cycloalkyl;
L1 is selected from the group consisting of O, NR3, CHR3, S and SO₂, or is absent;
B is mono- or bi-cyclic heteroaryl ring;
Y1 and Y2 are substituents of ring B independently selected from the group consisting of hydrogen, (C₁-C₄)alkyl, (C₁-C₄)haloalkyl, (C₁-C₄)alkoxy, (C₁-C₄)alkoxy-(C₁-C₄)alkoxy, hydroxy-(C₁-C₄)alkoxy, hydroxy-(C₁-C₄)alkyl, (C₁-C₄)alkyl-heterocycloalkyl-(C₀-C₄)alkoxy, (C₁-C₄)haloalkoxy, halogen, cyano, cyano-(C₁-C₄)alkyl, cyano-(C₁-C₄)alkyl-heterocycloalkyl, CONR1R2, NHCOR1, NR1R2, R1R2N—(C₁-C₄)alkyl, heterocycloalkyl, (C₁-C₄)alkyl-heterocycloalkyl, heterocycloalkyl-(C₁-C₄)alkyl, (C₁-C₄)alkyl-heterocycloalkyl-carbonyl, (C₀-C₄)alkyl-heterocycloalkyl-carbonyl, (C₁-C₄)alkyl-phenyl and monocyclic (C₁-C₄)alkyl-heteroaryl;
R1 and R2 are independently selected from the group consisting of hydrogen, (C₁-C₄)alkyl, (C₁-C₄)hydroxyalkyl, (C₁-C₄)alkoxy(C₁-C₄)alkyl, (C₁-C₄)alkylamino-(C₁-C₄)alkyl, di-(C₁-C₄)alkylamino-(C₁-C₄)alkyl, optionally substituted (C₃-C₆)cycloalkyl, optionally substituted heterocycloalkyl and optionally substituted heterocycloalkyl-(C₁-C₄)alkoxy, wherein optional substituents are one or more and are selected from (C₁-C₄)alkyl, (C₁-C₄)alkoxy, (C₁-C₄)haloalkyl and (C₁-C₄)haloalkoxy; and
R3 is selected from the group consisting of hydrogen and (C₁-C₄)alkyl;
or a stereoisomer, tautomer, solvate or pharmaceutically acceptable salt thereof.

15: A process for preparing the compound, or stereoisomer, tautomer, solvate or pharmaceutically acceptable salt thereof, according to claim 2, comprising reacting an intermediate compound of formula (XXXIII) and/or an intermediate compound of formula (XXXIX) and/or an intermediate compound of formula (XL): 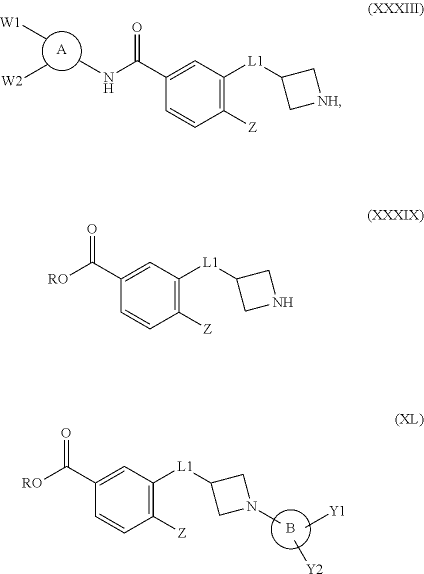
wherein
R is (C₁-C₄)alkyl,
A is a ring selected from the group consisting of: 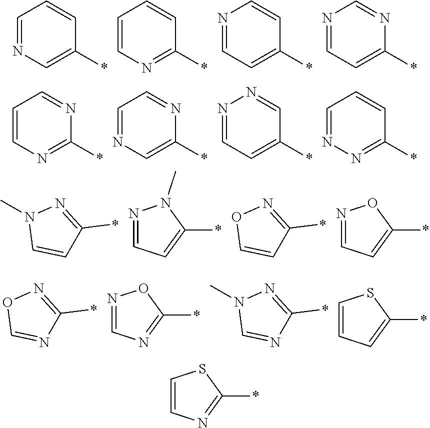
and phenyl, wherein [symbol] indicates a direct bond to NH;
W1 and W2 are substituents of ring A selected from the group consisting of hydrogen, halogen, (C₁-C₄)alkyl, (C₁-C₄)haloalkyl, (C₁-C₄)alkoxy, (C₁-C₄)haloalkoxy, R1R2N—(C₁-C₄)alkyl and (C₃-C₆)cycloalkyl;
Z is selected from the group consisting of (C₁-C₄)alkyl, (C₁-C₄)haloalkyl, (C₁-C₄)alkoxy, (C₁-C₄)haloalkoxy, (C₁-C₄)hydroxyalkyl and (C₃-C₆)cycloalkyl;
L1 is selected from the group consisting of O, NR3, CHR3, S and SO₂, or is absent;
B is mono- or bi-cyclic heteroaryl ring;
Y1 and Y2 are substituents of ring B independently selected from the group consisting of hydrogen, (C₁-C₄)alkyl, (C₁-C₄)haloalkyl, (C₁-C₄)alkoxy, (C₁-C₄)alkoxy-(C₁-C₄)alkoxy, hydroxy-(C₁-C₄)alkoxy, hydroxy-(C₁-C₄)alkyl, (C₁-C₄)alkyl-heterocycloalkyl-(C₀-C₄)alkoxy, (C₁-C₄)haloalkoxy, halogen, cyano, cyano-(C₁-C₄)alkyl, cyano-(C₁-C₄)alkyl-heterocycloalkyl, CONR1R2, NHCOR1, NR1R2, R1R2N—(C₁-C₄)alkyl, heterocycloalkyl, (C₁-C₄)alkyl-heterocycloalkyl, heterocycloalkyl-(C₁-C₄)alkyl, (C₁-C₄)alkyl-heterocycloalkyl-carbonyl, (C₀-C₄)alkyl-heterocycloalkyl-carbonyl, (C₁-C₄)alkyl-phenyl and monocyclic (C₁-C₄)alkyl-heteroaryl;
R1 and R2 are independently selected from the group consisting of hydrogen, (C₁-C₄)alkyl, (C₁-C₄)hydroxyalkyl, (C₁-C₄)alkoxy(C₁-C₄)alkyl, (C₁-C₄)alkylamino-(C₁-C₄)alkyl, di-(C₁-C₄)alkylamino-(C₁-C₄)alkyl, optionally substituted (C₃-C₆)cycloalkyl, optionally substituted heterocycloalkyl and optionally substituted heterocycloalkyl-(C₁-C₄)alkoxy, wherein optional substituents are one or more and are selected from (C₁-C₄)alkyl, (C₁-C₄)alkoxy, (C₁-C₄)haloalkyl and C₁-C₄)haloalkoxy; and
R3 is selected from the group consisting of hydrogen and (C₁-C₄)alkyl.
